(19) United States
(12) Patent Application Publication
(10) Pub. No.: US 2005/0180972 A1
(43) Pub. Date: Aug. 18, 2005

(54) Anti-CD20 antibody-drug conjugates for the treatment of cancer and immune disorders

(76) Inventors: Alan F. Wahl, Mercer Island, WA (US); Peter D. Senter, Seattle, WA (US); Che-Leung Law, North Shoreline, WA (US); Charles G. Cerveny, Seattle, WA (US)

Correspondence Address: JONES DAY, 222 EAST 41ST ST, NEW YORK, NY, 10017, US

(21) Appl. No.: 10/632,151

(22) Filed: Jul. 30, 2003

Related U.S. Application Data
(60) Provisional application No. 60/400,404, filed on Jul. 31, 2002.

(51) Int. Cl. A61K 39/395; C07K 16/46

(52) U.S. Cl. 424/144.1; 424/178.1; 530/391.1

(57) ABSTRACT

The present invention relates to methods and compositions for the treatment of CD20-expressing cancers and immune disorders involving CD20-expressing cells. The present methods comprise administering to a subject an anti CD20 antibody-drug conjugate that has a high potency and/or is capable of internalizing into CD20-expressing cells. The present invention further provides pharmaceutical compositions and kits comprising such conjugates. The present invention yet further provides methods of and compositions relating to combination therapy of cancer and immune disorders involving CD20-expressing cells using the anti-CD20 antibody-drug conjugates of the invention.

### RELATED APPLICATIONS

This application claims benefit of U.S. provisional application No.: 60/400,404, filed Jul. 31, 2002, which is incorporated herein by reference in its entirety.

### 1. FIELD OF THE INVENTION

The present invention relates to methods and compositions for the treatment of CD20-expressing cancers and immune disorders involving CD20-expressing cells. The present invention provides methods of treatment, comprising administering an anti CD20 antibody-drug conjugate that has a high potency and/or is capable of internalizing into CD20-expressing cells. The present invention further provides pharmaceutical compositions and kits comprising such conjugates.

### 2. BACKGROUND OF THE INVENTION

More than 270,000 people in the United States currently suffer from non-Hodgkin's lymphoma (NHL). In 2001, an estimated 56,200 new cases were diagnosed and 26,300 deaths were attributed to NHL in the United States (American Cancer Society Facts and FIGS. 2001). NHL is frequently treated with conventional chemotherapies as cyclophosphamide, fludarabine or chlorambucil or combination therapies such as as CVP or CHOP (El-Ismail et al., 1987, Eur. J. Cancer Clin. Oncol. 23:1379-84). Increasingly, NHL is being treated using Rituximab (RITUXAN®; IDEC Pharmaceuticals, San Diego, Calif., and Genentech, Inc., San Francisco, Calif.), a monoclonal antibody targeting the CD20 cell surface antigen. Rituximab was approved by the FDA for treatment of relapsed or refractory follicular lymphoma in November 1997 (Leget and Czuczman, 1998, Curr. Opin. Oncol. 10:548-551). Patients with relapsed low-grade or follicular lymphoma treated with weekly infusions of Rituximab showed a 48% response rate (6% complete responses, 42% partial responses) (Grillo-Lopez et al., 1999, Sentin Oncol. 26(5 Supp114):66-73.

The efficacy of Rituximab and other anti-CD20 mAbs has been increased in two ways: First, by conjugation of the anti-CD20 mAb to radioisotopes (e.g., Yttrium⁹⁰-labeled Ibritumomab (trade name Zevalin®), which has been approved by the FDA; and iodinated mAb B-1 (¹³¹I -tositumomab, trade name Bexxar®), which is currently under FDA review for clinical use (Wagner et al., 2002, J. Nucl Med. 43:267-272)). Second, patient outcome can be improved by the use of Rituximab in combination with standard cytotoxic therapies such as CHOP (Coiffier et al., 2002, N. Engl. J. Med. 346:235-242). In intermediate-grade NHL, Rituximab in combination with CHOP chemotherapy increased complete response (CR) rate from 60% to 75%, prolonged 1 year event-free survival from 49% to 69% and increased overall survival from 68% to 83% as compared to CHOP alone (Grillo-Lopez, 2001; Curr. Pharm. Biotechnol. 2:301-311). However, although radiolabeling and combination with chemotherapy may improve the efficacy of anti-CD20 therapeutics, these approaches are associated with undesirable side effects. For example, isotope therapy is associated with undesirable myelosuppression (Witzig, 2001, Cancer Chemother Pharmacol 48 (Suppl 1):S91-5), and combination therapy with antibodies and chemotherapeutics is associated with immunosuppression. Further, isotopically labeled substances are difficult to produce. And often patients experience relapse after initial treatment with isotopically labeled substances.

The anti-CD20 monoclonal antibody Rituximab has been successfully used to treat immune disorders (Perrotta et al, 2002, Br J Haematol 116(2):465-467; Zaja et al, 2002, Haematologica 87(2):189-195; Quartier et al, 2001, Lancet 358(9292):1511-1513; Aranda et al, 2002, Transplantation 73(6):907-910).

An effective approach to enhancing the efficacy of anti-cancer therapeutics is by linking of cytotoxic drugs or toxins to mAbs that are capable of being internalized by a target cell. These agents are termed antibody-drug conjugates (ADCs) and immunotoxins, respectively. Upon administration to a patient, ADCs and immunotoxins bind to target cells through their antibody portions and become internalized, allowing the drugs or toxins to exert their cytotoxic or cytostatic effects.

Anti-CD20 mAbs have been evaluated in at least two systems to determine if linking the mAb to a cytotoxic drug or toxin increased their anti-tumor efficacy. First, an ADC composed of the anti-CD20 mAb 2H7 chemically conjugated to the anti-cancer agent doxorubicin was produced and tested. Braslawsky et al. observed that an anti-CD20-antibody doxorubicin conjugate was not cytotoxic (Braslawsky et al., 1991, Cancer Immunol Immunother. 33:367-374). Similarly, anti-CD20 mAbs conjugated to the protein toxin ricin were tested on antigen positive cells. Anti-CD20-ricin immunotoxin conjugates were unable to block protein synthesis and induce calcium mobilization on CD20 positive cells whereas a comparably conjugated anti-CD19 conjugate was found to be effective against CD19 positive cells (Goulet et al., 1997, Blood; 90(6):2364-2375). Radiolabeled mAbs to CD19 and CD20 demonstrated that CD19 could induce modulation and internalization of mAb/Ag complexes, however a comparable effect was not observed with isotopically labeled CD20 mAb (Vervoordeldonk et al., 1994, Cancer 73(3 Suppl):1006-11). These data suggested that anti-CD20 mAbs were unlikely to be effective targeting agents of drugs or toxins that act intracellularly, and therefore were ineffective components of ADCs or immunotoxins.

Accordingly, there is a need for anti-CD20-containing ADCs and immunoconjugates that are constructed in such a manner so as to be capable exerting a clinically useful cytotoxic or cytostatic effect on CD20-expressing cells, for example by being internalized into CD20-expressing cells at a rate sufficient to exert such a clinically useful cytotoxic or cytostatic effect. Such compounds would be useful therapeutic agents against cancers that express CD20 or immune disorders that are mediated by CD20-expressing cells without the side effects of myelosuppression or immunosuppression seen using radiolabeled antibodies or combination therapy.

Citation or identification of any reference herein shall not be construed as an admission that such reference is available as prior art to the present invention.

### 3. SUMMARY OF THE INVENTION

The present invention provides anti-CD20 antibody-cytotoxic agent conjugates comprising anti-CD20 antibodies conjugated to cytotoxic agents that have a high potency and/or are capable of promoting accumulation of the anti-CD20 ADC into CD20-expressing cells. Generally, the antibody unit of an anti-CD20 antibody-cytotoxic agent conjugate of the invention is preferably conjugated to the cytotoxic agent via a linker. The present invention yet further provides methods of treatment of CD20-expressing cancers and immune disorders involving CD20-expressing cells, comprising administering to a patient in need of such treatment an anti-CD20 antibody-cytotoxic agent conjugate of the invention, in either single therapy or combination therapy regimens. The present invention further provides pharmaceutical compositions and kits comprising such conjugates. The remainder of this section describes specific embodiments of the anti-CD20 antibody-cytotoxic agent conjugates of the invention, pharmaceutical compositions and comprising these conjugates and methods of their use.

The present invention provides an anti-CD20 antibody-cytotoxic agent conjugate, wherein the cytotoxic agent of the anti-CD20 antibody-cytotoxic agent conjugate has an IC₅₀ of at least 40-fold less than the IC₅₀ of doxorubicin, and wherein the IC₅₀ of each of the cytotoxic agent and doxorubicin is measured by a method comprising: (a) culturing one or more CD20-expressing cell populations in the presence of one or more concentrations of the cytotoxic agent for a 72- to 96-hour period; (b) culturing one or more CD20-expressing cell populations in the presence of one or more concentrations of doxorubicin for a 72- to 96-hour period; and (c) identifying a concentration of the cytotoxic agent and doxorubicin, respectively, at which 50% fewer cells in the CD20-expressing cell populations of steps (a) and (b), respectively, are viable at the end of the period relative to a CD20-expressing cell population cultured in the absence of the cytotoxic agent and doxorubicin, wherein the CD20-expressing cell populations of steps (a), (b) and (c) are of the same cell type and are cultured under the same conditions, and wherein the concentration of the cytotoxic agent and doxorubicin identified in step (c) is the IC₅₀ of the cytotoxic agent and doxorubicin, respectively. In certain embodiments, the IC₅₀ of the cytotoxic agent is between 40-fold and 4,000-fold less than the IC₅₀ of doxorubicin. In certain embodiments, the IC₅₀ of the cytotoxic agent is between 100-fold and 1,000-fold less than the IC₅₀ of doxorubicin. In certain embodiments, the IC₅₀ of the cytotoxic agent is between 50-fold and 200-fold less than the IC₅₀ of doxorubicin. In certain embodiments, the IC₅₀ of the cytotoxic agent is between 400-fold and 600-fold less than the IC₅₀ of doxorubicin. In certain embodiments, the IC₅₀ of the cytotoxic agent is between 800-fold and 1,200-fold less than the IC₅₀ of doxorubicin. In certain embodiments, the IC₅₀ of the cytotoxic agent is at least 50-fold less than the IC₅₀ of doxorubicin. In certain embodiments, the IC₅₀ of the cytotoxic agent is at least 60-fold less than the IC₅₀ of doxorubicin. In certain embodiments, the IC₅₀ of the cytotoxic agent is at least 70-fold less than the IC₅₀ of doxorubicin. In certain embodiments, the IC₅₀ of the cytotoxic agent is at least 80-fold less than the IC₅₀ of doxorubicin. In certain embodiments, the IC₅₀ of the cytotoxic agent is at least 90-fold less than the IC₅₀ of doxorubicin. In certain embodiments, the IC₅₀ of the cytotoxic agent is at least 100-fold less than the IC₅₀ of doxorubicin. In certain embodiments, the IC₅₀ of the cytotoxic agent is at least 125-fold less than the IC₅₀ of doxorubicin. In certain embodiments, the IC₅₀ of the cytotoxic agent is at least 150-fold less than the IC₅₀ of doxorubicin. In certain embodiments, the IC₅₀ of the cytotoxic agent is at least 175-fold less than the IC₅₀ of doxorubicin. In certain embodiments, the IC₅₀ of the cytotoxic agent is at least 200-fold less than the IC₅₀ of doxorubicin. In certain embodiments, the IC₅₀ of the cytotoxic agent is at least 2,000-fold less than the IC₅₀ of doxorubicin. In certain embodiments, the IC₅₀ of the cytotoxic agent is no more than 500-fold less than the IC₅₀ of doxorubicin. In certain embodiments, the IC₅₀ of the cytotoxic agent is no more than 600-fold less than the IC₅₀ of doxorubicin. In certain embodiments, the IC₅₀ of the cytotoxic agent is no more than 700-fold less than the IC₅₀ of doxorubicin. In certain embodiments, the IC₅₀ of the cytotoxic agent is no more than 1000-fold less than the IC₅₀ of doxorubicin. In certain embodiments, the IC₅₀ of the cytotoxic agent is no more than 2000-fold less than the IC₅₀ of doxorubicin. In certain embodiments, the CD20-expressing cell population is a population of Daudi cells, Ramos cells, Raji cells, IM-9 cells, HS-Sultan cells, ARH-77 cells, HT cells, RL cells, DB cells, or 295R cells.

The invention further provides an anti-CD20 antibody-cytotoxic agent conjugate, wherein the anti-CD20 antibody-cytotoxic agent conjugate has an IC₅₀ of at least 40-fold less than the IC₅₀ of an anti-CD20 antibody-doxorubicin conjugate, wherein the anti-CD20 antibody-cytotoxic agent conjugate and the anti-CD20 antibody-doxorubicin conjugate comprise the same anti-CD20 antibody, and wherein the IC₅₀ of each of the anti-CD20 antibody-cytotoxic agent conjugate and the anti-CD20 antibody-doxorubicin conjugate is measured by a method comprising: (a) culturing one or more CD20-expressing cell populations in the presence of one or more concentrations of the anti-CD20 antibody-cytotoxic agent conjugate for a 72- to 96-hour period; (b) culturing one or more CD20-expressing cell populations in the presence of one or more concentrations of the anti-CD20 antibody-doxorubicin conjugate for a 72- to 96-hour period; and (c) identifying a concentration of the anti-CD20 antibody-cytotoxic agent conjugate and the anti-CD20 antibody-doxorubicin conjugate, respectively, at which 50% fewer cells in the CD20-expressing cell populations of steps (a) and (b), respectively, are viable at the end of the period relative to a CD20-expressing cell population type cultured in the absence of the anti-CD20 antibody-cytotoxic agent conjugate and the anti-CD20 antibody-doxorubicin conjugate, wherein the CD20-expressing cell populations of steps (a), (b) and (c) are of the same cell type and are cultured under the same conditions, and wherein the concentration of the anti-CD20 antibody-cytotoxic agent conjugate and the anti-CD20 antibody-doxorubicin conjugate identified in step (c) is the IC₅₀ of the anti-CD20 antibody-cytotoxic agent conjugate and the anti-CD20 antibody-doxorubicin conjugate, respectively. In certain embodiments, the IC₅₀ of the cytotoxic agent is between 40-fold and 4,000-fold less than the IC₅₀ of doxorubicin. In certain embodiments, the IC₅₀ of the cytotoxic agent is between 100-fold and 1,000-fold less than the IC₅₀ of doxorubicin. In certain embodiments, the IC₅₀ of the cytotoxic agent is between 50-fold and 200-fold less than the IC₅₀ of doxorubicin. In certain embodiments, the IC₅₀ of the cytotoxic agent is between 400-fold and 600-fold less than the IC₅₀ of doxorubicin. In certain embodiments, the IC₅₀ of the cytotoxic agent is between 800-fold and 1,200-fold less than the IC₅₀ of doxorubicin. In certain embodiments, the IC₅₀ of the anti-CD20 antibody-cytotoxic agent conjugate is at least 50-fold less than the IC₅₀ of the anti-CD20 antibody-doxorubicin conjugate. In certain embodiments, the IC₅₀ of the anti-CD20 antibody-cytotoxic agent conjugate is at least 60-fold less than the IC₅₀ of the anti-CD20 antibody-doxorubicin conjugate. In certain embodiments, the IC₅₀ of the anti-CD20 antibody-cytotoxic agent conjugate is at least 70-fold less than the IC₅₀ of the anti-CD20 antibody-doxorubicin conjugate. In certain embodiments, the IC₅₀ of the anti-CD20 antibody-cytotoxic agent conjugate is at least 80-fold less than the IC₅₀ of the anti-CD20 antibody-doxorubicin conjugate. In certain embodiments, the IC₅₀ of the anti-CD20 antibody-cytotoxic agent conjugate is at least 90-fold less than the IC₅₀ of the anti-CD20 antibody-doxorubicin conjugate. In certain embodiments, the IC₅₀ of the anti-CD20 antibody-cytotoxic agent conjugate is at least 100-fold less than the IC₅₀ of the anti-CD20 antibody-doxorubicin conjugate. In certain embodiments, the IC₅₀ of the anti-CD20 antibody-cytotoxic agent conjugate is at least 125-fold less than the IC₅₀ of the anti-CD20 antibody-doxorubicin conjugate. In certain embodiments, the IC₅₀ of the anti-CD20 antibody-cytotoxic agent conjugate is at least 150-fold less than the IC₅₀ of the anti-CD20 antibody-doxorubicin conjugate. In certain embodiments, the IC₅₀ of the anti-CD20 antibody-cytotoxic agent conjugate is at least 175-fold less than the IC₅₀ of the anti-CD20 antibody-doxorubicin conjugate. In certain embodiments, the IC₅₀ of the anti-CD20 antibody-cytotoxic agent conjugate is at least 200-fold less than the IC₅₀ of the anti-CD20 antibody-doxorubicin conjugate. In certain embodiments, the IC₅₀ of the anti-CD20 antibody-cytotoxic agent conjugate is at least 2,000-fold less than the IC₅₀ of the anti-CD20 antibody-doxorubicin conjugate. In certain embodiments, the IC₅₀ of the anti-CD20 antibody-cytotoxic agent conjugate is no more than 500-fold less than the IC₅₀ of the anti-CD20 antibody-doxorubicin conjugate. In certain embodiments, the IC₅₀ of the anti-CD20 antibody-cytotoxic agent conjugate is no more than 600-fold less than the IC₅₀ of the anti-CD20 antibody-doxorubicin conjugate. In certain embodiments, the IC₅₀ of the anti-CD20 antibody-cytotoxic agent conjugate is no more than 700-fold less than the IC₅₀ of the anti-CD20 antibody-doxorubicin conjugate. In certain embodiments, the IC₅₀ of the anti-CD20 antibody-cytotoxic agent conjugate is no more than 1000-fold less than the IC₅₀ of the anti-CD20 antibody-doxorubicin conjugate. In certain embodiments, the IC₅₀ of the anti-CD20 antibody-cytotoxic agent conjugate is no more than 2000-fold less than the IC₅₀ of the anti-CD20 antibody-doxorubicin conjugate. In certain embodiments, the CD20-expressing cell population is a population of Daudi cells, Ramos cells, Raji cells, IM-9 cells, HS-Sultan cells, ARH-77 cells, HT cells, RL cells, DB cells, or 295R cells.

The invention further provides an anti-CD20 antibody-cytotoxic agent conjugate, wherein the conjugate has a rate of accumulation in a CD20-expressing cell that is at least 20-fold greater than the rate of accumulation of an unconjugated form of the anti-CD20 antibody in the CD20-expressing cell, wherein the rates of accumulation of the conjugate and of the unconjugated form of the antibody are measured by a method comprising: (a) culturing a population of the CD20-expressing cell with the conjugate; (b) culturing a population of the CD20-expressing cell with the unconjugated antibody, wherein the populations of steps (a) and (b) are cultured under the same conditions; and (c) measuring the amount of the conjugate and unconjugated antibody accumulated in the populations of steps (a) and (b), respectively. In certain embodiments, the rates of accumulation of the conjugate and the unconjugated form of the antibody in the CD20-expressing cell are determined by: (a) culturing a population of the CD20-expressing cell in the presence of the conjugate, wherein the antibody portion of the conjugate is labeled with a radioactive isotope; (b) culturing a population of the CD20-expressing cell with the unconjugated form of the antibody under the same conditions as the culturing of step (a), wherein the unconjugated form of the antibody is labeled with the radioactive isotope; (c) washing each of the populations of steps (a) and (b) under acidic conditions; and (d) comparing the amount of the radioactive isotope in the populations of steps (a) and (b) after the washing of step (c), wherein the rate of accumulation of the conjugate in the CD20-expressing cell is at least 20-fold greater than the rate of accumulation of the unconjugated form of the anti-CD20 antibody in the CD20-expressing cell if the amount of the radioactive isotope in the population of step (a) is at least 20-fold greater than the amount of the radioactive isotope in the population of step (b). In certain embodiments, the CD20-expressing cell is a Daudi cell, a Ramos cell, a Raji cell, an IM-9 cell, a HS-Sultan cell, an ARH-77 cell, a HT cell, a RL cell, a DB cell, or a 295R cell. In certain embodiments, the conjugate has a rate of accumulation inside the CD20-expressing cell that is between 20-fold and 5,000-fold greater than the rate of accumulation inside the CD20-expressing cell of the anti-CD20 antibody in unconjugated form. In certain embodiments, the conjugate has a rate of accumulation inside the CD20-expressing cell that is between 100-fold and 1,000-fold greater than the rate of accumulation inside the CD20-expressing cell of the anti-CD20 antibody in unconjugated form. In certain embodiments, the conjugate has a rate of accumulation inside the CD20-expressing cell that is between 25-fold and 75-fold greater than the rate of accumulation inside the CD20-expressing cell of the anti-CD20 antibody in unconjugated form. In certain embodiments, the conjugate has a rate of accumulation inside the CD20-expressing cell that is at least 50-fold greater than the rate of accumulation inside the CD20-expressing cell of the anti-CD20 antibody in unconjugated form. In certain embodiments, the conjugate has a rate of accumulation inside the CD20-expressing cell that is at least 200-fold greater than the rate of accumulation inside the CD20-expressing cell of the anti-CD20 antibody in unconjugated form. In certain embodiments, the conjugate has a rate of accumulation inside the CD20-expressing cell that is at least 500-fold greater than the rate of accumulation inside the CD20-expressing cell of the anti-CD20 antibody in unconjugated form. In certain embodiments, the conjugate has a rate of accumulation inside the CD20-expressing cell that is at least 1000-fold greater than the rate of accumulation inside the CD20-expressing cell of the anti-CD20 antibody in unconjugated form. In certain embodiments, the conjugate has a rate of accumulation inside the CD20-expressing cell that is no more than 200-fold greater than the rate of accumulation inside the CD20-expressing cell of the anti-CD20 antibody in unconjugated form. In certain embodiments, the conjugate has a rate of accumulation inside the CD20-expressing cell that is no more than 1000-fold greater than the rate of accumulation inside the CD20-expressing cell of the anti-CD20 antibody in unconjugated form. In certain embodiments, the conjugate has a rate of accumulation inside the CD20-expressing cell that is no more than 2000-fold greater than the rate of accumulation inside the CD20-expressing cell of the anti-CD20 antibody in unconjugated form.

The invention further provides an anti-CD20 antibody-cytotoxic agent conjugate, wherein the conjugate has a rate of accumulation in a CD20-expressing cell that is at least 20-fold greater than the rate of accumulation of an anti-CD20 antibody-doxorubicin conjugate in a CD20-expressing cell of the same cell type, wherein the anti-CD20 antibody-cytotoxic agent conjugate and the anti-CD20 antibody-doxorubicin conjugate comprise the same anti-CD20 antibody, and wherein the rates of accumulation of the anti-CD20 antibody-cytotoxic agent conjugate and of the anti-CD20 antibody-doxorubicin conjugate are measured by a method comprising: (a) culturing a population of the CD20-expressing cell with the anti-CD20 antibody-cytotoxic agent conjugate; (b) culturing a population of the CD20-expressing cell with the anti-CD20 antibody-doxorubicin conjugate, wherein the populations of steps (a) and (b) are cultured under the same conditions; and (c) measuring the amount of the anti-CD20 antibody-cytotoxic agent conjugate and anti-CD20 antibody-doxorubicin conjugate accumulated in the populations of steps (a) and (b), respectively. In certain embodiments, the conjugate has a rate of accumulation inside the CD20-expressing cell that is at least 50-fold greater than the rate of accumulation inside the CD20-expressing cell of the anti-CD20 antibody-doxorubicin conjugate. In certain embodiments, the conjugate has a rate of accumulation inside the CD20-expressing cell that is between 20-fold and 5,000-fold greater than the rate of accumulation inside the CD20-expressing cell of the anti-CD20 antibody-doxorubicin conjugate. In certain embodiments, the conjugate has a rate of accumulation inside the CD20-expressing cell that is between 50-fold and 2,500-fold greater than the rate of accumulation inside the CD20-expressing cell of the anti-CD20 antibody-doxorubicin conjugate. In certain embodiments, the conjugate has a rate of accumulation inside the CD20-expressing cell that is between 100-fold and 1,000-fold greater than the rate of accumulation inside the CD20-expressing cell of the anti-CD20 antibody-doxorubicin conjugate. In certain embodiments, the conjugate has a rate of accumulation inside the CD20-expressing cell that is between 25-fold and 75-fold greater than the rate of accumulation inside the CD20-expressing cell of the anti-CD20 antibody-doxorubicin conjugate. In certain embodiments, the conjugate has a rate of accumulation inside the CD20-expressing cell that is at least 200-fold greater than the rate of accumulation inside the CD20-expressing cell of the anti-CD20 antibody-doxorubicin conjugate. In certain embodiments, the conjugate has a rate of accumulation inside the CD20-expressing cell that is at least 1000-fold greater than the rate of accumulation inside the CD20-expressing cell of the anti-CD20 antibody-doxorubicin conjugate. In certain embodiments, the conjugate has a rate of accumulation inside the CD20-expressing cell that is no more than 200-fold greater than the rate of accumulation inside the CD20-expressing cell of the anti-CD20 antibody-doxorubicin conjugate. In certain embodiments, the conjugate has a rate of accumulation inside the CD20-expressing cell that is no more than 1000-fold greater than the rate of accumulation inside the CD20-expressing cell of the anti-CD20 antibody-doxorubicin conjugate. In certain embodiments, the conjugate has a rate of accumulation inside the CD20-expressing cell that is no more than 2000-fold greater than the rate of accumulation inside the CD20-expressing cell of the anti-CD20 antibody-doxorubicin conjugate.

In certain embodiments, the invention provides an anti-CD20 antibody-cytotoxic agent conjugate, wherein the conjugate exhibits an at least 1.5-fold greater accumulation in a non-peripheral region inside a CD20-expressing cell than the accumulation of an unconjugated form of the anti-CD20 antibody in the CD20-expressing cell, wherein the accumulation of the conjugate and of the unconjugated form of the antibody are measured by a method comprising: (a) culturing a population of the CD20-expressing cell with the conjugate; (b) culturing a population of the CD20-expressing cell with the unconjugated form of the anti-CD20 antibody; and (c) detecting by confocal fluorescence microscopy localization of the conjugate and the unconjugated form of the anti-CD20 antibody in the populations of steps (a) and (b), respectively, wherein the populations of steps (a) and (b) are cultured under the same conditions and for the same period of time, and wherein the conjugate exhibits an at least 1.5-fold greater accumulation in the CD20-expressing cell than the accumulation of the unconjugated form of the anti-CD20 antibody in the CD20-expressing cell if: (i) at least 1.5-fold as many cells of the population of step (a) contain a detectable amount of the conjugate in a non-peripheral region as the number of cells of the population of step (b) contain the unconjugated form of the antibody in a non-peripheral region; or (ii) the accumulation of the conjugate in a non-peripheral region of the majority of CD20-expressing cells of the population of step (a) is at least 1.5-fold greater than the accumulation of the unconjugated form of the anti-CD20 antibody in the majority of CD20-expressing cells of the population of step (b). In certain embodiments, the conjugate exhibits an at least 2-fold greater accumulation in the CD20-expressing cell than the accumulation of the unconjugated form of the anti-CD20 antibody in the CD20-expressing cell. In certain embodiments, the conjugate exhibits a between 1.5-fold and 5,000-fold greater accumulation in the CD20-expressing cell than the accumulation of the unconjugated form of the anti-CD20 antibody in the CD20-expressing cell. In certain embodiments, the conjugate exhibits a between 5-fold and 2,500-fold greater accumulation in the CD20-expressing cell than the accumulation of the unconjugated form of the anti-CD20 antibody in the CD20-expressing cell. In certain embodiments, the conjugate exhibits a between 50-fold and 1,000-fold greater accumulation in the CD20-expressing cell than the accumulation of the unconjugated form of the anti-CD20 antibody in the CD20-expressing cell. In certain embodiments, the conjugate exhibits a between 100-fold and 500-fold greater accumulation in the CD20-expressing cell than the accumulation of the unconjugated form of the anti-CD20 antibody in the CD20-expressing cell. In certain embodiments, the conjugate exhibits an at least 5-fold greater accumulation in the CD20-expressing cell than the accumulation of the unconjugated form of the anti-CD20 antibody in the CD20-expressing cell. In certain embodiments, the conjugate exhibits an at least 20-fold greater accumulation in the CD20-expressing cell than the accumulation of the unconjugated form of the anti-CD20 antibody in the CD20-expressing cell. In certain embodiments, the conjugate exhibits an at least 50-fold greater accumulation in the CD20-expressing cell than the accumulation of the unconjugated form of the anti-CD20 antibody in the CD20-expressing cell. In certain embodiments, the conjugate exhibits an at least 500-fold greater accumulation in the CD20-expressing cell than the accumulation of the unconjugated form of the anti-CD20 antibody in the CD20-expressing cell. In certain embodiments, the conjugate exhibits an at least 5000-fold greater accumulation in the CD20-expressing cell than the accumulation of the unconjugated form of the anti-CD20 antibody in the CD20-expressing cell. In certain embodiments, the conjugate exhibits an at most 50-fold greater accumulation in the CD20-expressing cell than the accumulation of the unconjugated form of the anti-CD20 antibody in the CD20-expressing cell. In certain embodiments, the conjugate exhibits an at most 500-fold greater accumulation in the CD20-expressing cell than the accumulation of the unconjugated form of the anti-CD20 antibody in the CD20-expressing cell. In certain embodiments, the conjugate exhibits an at most 5,000-fold greater accumulation in the CD20-expressing cell than the accumulation of the unconjugated form of the anti-CD20 antibody in the CD20-expressing cell. In certain embodiments, the majority of CD20-expressing cells of the population of step (b) is at least 60% of the cells in the population. In certain embodiments, the majority of CD20-expressing cells of the population of step (b) is at least 70% of the cells in the population. In certain embodiments, the majority of CD20-expressing cells of the population of step (b) is at least 80% of the cells in the population. In certain embodiments, the CD20-expressing cell is a Daudi cell, a Ramos cell, a Raji cell, an IM-9 cell, a HS-Sultan cell, an ARH-77 cell, a HT cell, a RL cell, a DB cell, or a 295R cell.

The invention further provides, an anti-CD20 antibody-cytotoxic agent conjugate, wherein the conjugate exhibits an at least 1.5-fold greater accumulation in a non-peripheral region inside a CD20-expressing cell than the accumulation of an anti-CD20 antibody-doxorubicin conjugate, wherein the anti-CD20 antibody-cytotoxic agent conjugate and the anti-CD20 antibody-doxorubicin conjugate comprise the same anti-CD20 antibody, in the CD20-expressing cell, wherein the accumulation of the conjugate and of the anti-CD20 antibody-doxorubicin conjugate are measured by a method comprising: (a) culturing a population of the CD20-expressing cell with the conjugate; (b) culturing a population of the CD20-expressing cell with the anti-CD20 antibody-doxorubicin conjugate; and (c) detecting by confocal fluorescence microscopy localization of the conjugate and the anti-CD20 antibody-doxorubicin conjugate in the populations of steps (a) and (b), respectively, wherein the populations of steps (a) and (b) are cultured under the same conditions and for the same period of time, and wherein the conjugate exhibits an at least 1.5-fold greater accumulation in the CD20-expressing cell than the accumulation of the anti-CD20 antibody-doxorubicin conjugate in the CD20-expressing cell if: (i) at least 1.5-fold as many cells of the population of step (a) contain a detectable amount of the conjugate in a non-peripheral region as the number of cells of the population of step (b) contain the anti-CD20 antibody-doxorubicin conjugate in a non-peripheral region; or (ii) the accumulation of the conjugate in a non-peripheral region of the majority of CD20-expressing cells of the population of step (a) is at least 1.5-fold greater than the accumulation of the anti-CD20 antibody-doxorubicin conjugate in the majority of CD20-expressing cells of the population of step (b). In certain embodiments, the conjugate exhibits an at least 2-fold greater accumulation in the CD20-expressing cell than the accumulation of the anti-CD20 antibody-doxorubicin conjugate in the CD20-expressing cell. In certain embodiments, the conjugate exhibits a between 1.5-fold and 5,000-fold greater accumulation in the CD20-expressing cell than the accumulation of the anti-CD20 antibody-doxorubicin conjugate in the CD20-expressing cell. In certain embodiments, the conjugate exhibits a between 5-fold and 2,500-fold greater accumulation in the CD20-expressing cell than the accumulation of the anti-CD20 antibody-doxorubicin conjugate in the CD20-expressing cell. In certain embodiments, the conjugate exhibits a between 50-fold and 1,000-fold greater accumulation in the CD20-expressing cell than the accumulation of the anti-CD20 antibody-doxorubicin conjugate in the CD20-expressing cell. In certain embodiments, the conjugate exhibits a between 100-fold and 500-fold greater accumulation in the CD20-expressing cell than the accumulation of the anti-CD20 antibody-doxorubicin conjugate in the CD20-expressing cell. In certain embodiments, the conjugate exhibits an at least 5-fold greater accumulation in the CD20-expressing cell than the accumulation of the anti-CD20 antibody-doxorubicin conjugate in the CD20-expressing cell. In certain embodiments, the conjugate exhibits an at least 20-fold greater accumulation in the CD20-expressing cell than the accumulation of the anti-CD20 antibody-doxorubicin conjugate in the CD20-expressing cell. In certain embodiments, the conjugate exhibits an at least 50-fold greater accumulation in the CD20-expressing cell than the accumulation of the anti-CD20 antibody-doxorubicin conjugate in the CD20-expressing cell. In certain embodiments, the conjugate exhibits an at least 200-fold greater accumulation in the CD20-expressing cell than the accumulation of the anti-CD20 antibody-doxorubicin conjugate in the CD20-expressing cell. In certain embodiments, the conjugate exhibits an at least 500-fold greater accumulation in the CD20-expressing cell than the accumulation of the anti-CD20 antibody-doxorubicin conjugate in the CD20-expressing cell. In certain embodiments, the conjugate exhibits an at least 2000-fold greater accumulation in the CD20-expressing cell than the accumulation of the anti-CD20 antibody-doxorubicin conjugate in the CD20-expressing cell. In certain embodiments, the conjugate exhibits an at most 50-fold greater accumulation in the CD20-expressing cell than the accumulation of the anti-CD20 antibody-doxorubicin conjugate in the CD20-expressing cell. In certain embodiments, the conjugate exhibits an at most 500-fold greater accumulation in the CD20-expressing cell than the accumulation of the anti-CD20 antibody-doxorubicin conjugate in the CD20-expressing cell. In certain embodiments, the conjugate exhibits an at most 5,000-fold greater accumulation in the CD20-expressing cell than the accumulation of the anti-CD20 antibody-doxorubicin conjugate in the CD20-expressing cell. In certain embodiments, the majority of CD20-expressing cells of the population of step (b) is at least 60% of the cells in the population. In certain embodiments, the majority of CD20-expressing cells of the population of step (b) is at least 70% of the cells in the population. In certain embodiments, the majority of CD20-expressing cells of the population of step (b) is at least 80% of the cells in the population. In certain embodiments, the CD20-expressing cell is a Daudi cell, a Ramos cell, a Raji cell, an IM-9 cell, a HS-Sultan cell, an ARH-77 cell, a HT cell, a RL cell, a DB cell, or a 295R cell.

In certain embodiments, the anti-CD20 antibody-cytotoxic agent conjugate and the conjugate of the anti-CD20 antibody and doxorubicin comprise the same linker.

In certain embodiments, the cytotoxic agent of an anti-CD20 antibody-cytotoxic agent conjugate of the invention is selected from the group consisting of an enediyne, a lexitropsin, a duocarmycin, a taxane, a puromycin, a dolastatin, a maytansinoid, and a vincaalkaloid. In certain, more specific embodiments, the cytotoxic agent is paclitaxel, docetaxel, CC-1065, SN-38, topotecan, morpholino-doxorubicin, rhizoxin, cyanomorpholino-doxorubicin, dolastatin-10, echinomycin, combretastatin, calicheamicin, maytansine, DM-1, auristatin E, auristatin EB, auristatin E-FP, monomethyl auristatin E, or netropsin.

In certain embodiments, the cytotoxic agent of an anti-CD20 antibody-cytotoxic agent conjugate of the invention is an anti-tubulin agent. In more specific embodiments, the cytotoxic agent is selected from the group consisting of a vinca alkaloid, a podophyllotoxin, a taxane, a baccatin derivative, a cryptophysin, a maytansinoid, a combretastatin, and a dolastatin. In more specific embodiments, the cytotoxic agent is vincristine, vinblastine, vindesine, vinorelbine, VP-16, camptothecin, paclitaxel, docetaxel, epithilone A, epithilone B, nocodazole, colchicine, colcimid, estramustine, cemadotin, discodermolide, maytansine, DM-1, auristatin E-FP, auristatin E, auristatine EB, monomethyl auristatin E or eleutherobin.

In a specific embodiment, the cytotoxic agent of an anti-CD20 antibody-cytotoxic agent conjugate of the invention is monomethyl Auristatin E (MMAE).

In specific embodiments, the anti-CD20 antibody of an anti-CD20 antibody-cytotoxic agent conjugate of the invention is conjugated to the cytotoxic agent via a linker, wherein the linker is peptide linker. In specific embodiments, the anti-CD20 antibody of an anti-CD20 antibody-cytotoxic agent conjugate of the invention is conjugated to the cytotoxic agent via a linker, wherein the linker is a val-cit linker, a phe-lys linker, a hydrazone linker, or a disulfide linker. In certain embodiments, the anti-CD20 antibody of an anti-CD20 antibody-cytotoxic agent conjugate of the invention is conjugated to the cytotoxic agent via a peptide linker.

In a specific embodiments, the conjugate of the invention is Rituximab-valine-citrulline-monomethyl Auristatin E (Rituximab-valcitMMAE or Rituximab-vcMMAE).

In certain embodiments, the anti-CD20 antibody of an anti-CD20 antibody-cytotoxic agent conjugate of the invention is conjugated to the cytotoxic agent via a linker, wherein the linker is hydrolyzable at a pH of less than 5.5. In a specific embodiment the linker is hydrolyzable at a pH of less than 5.0.

In certain embodiments, the anti-CD20 antibody of an anti-CD20 antibody-cytotoxic agent conjugate of the invention is conjugated to the cytotoxic agent via a linker, wherein the linker is cleavable by a protease. In a specific embodiment, the protease is a lysosomal protease. In other specific embodiments, the protease is, inter alia, a membrane-associated protease, an intracellular protease, or an endosomal protease.

In certain embodiments, the anti-CD20 antibody of an anti-CD20 antibody-cytotoxic agent conjugate of the invention is a monoclonal antibody, a humanized chimeric antibody, a chimeric antibody, a humanized antibody, a glycosylated antibody, a multispecific antibody, a human antibody, a single-chain antibody, a Fab fragment, a F(ab′) fragment, a F(ab′)₂ fragment, a Fd, a single-chain Fv, a disulfide-linked Fv, a fragment comprising a V_{L} domain, or a fragment comprising a V_{H} domain. In certain embodiments, the anti-CD20 antibody of an anti-CD20 antibody-cytotoxic agent conjugate of the invention is a polypeptide that binds specifically to CD20. In certain embodiments, the antibody is a bispecific antibody. In other embodiments, the antibody is not a bispecific antibody.

In certain embodiments, the anti-CD20 antibody is radioactively labeled. In certain embodiments, the anti-CD20 antibody of the anti-CD20 antibody-cytotoxic agent conjugate is radioactively labeled. In specific embodiments, the radioacive label is ⁹⁰Y, ¹¹¹In, ²¹¹At, 131I, ²¹²Bi, ²¹³Bi, ²²⁵Ac, ¹⁸⁶Re, ¹⁸⁸Re, ¹⁰⁹Pd, ⁶⁷Cu, ⁷⁷Br, ¹⁰⁵Rh, 198Au, ¹⁹⁹Au or ²¹²Pb.

In certain embodiments, the anti-CD20 antibody of an anti-CD20 antibody-cytotoxic agent conjugate of the invention comprises one or more CDRs of C2B8, 1F5, FB1, 2H7, 93-1B3, 109-3C2, B1, B9E9, 7D1, H147, L26, L27, or MEM97. In certain more specific embodiments, such an anti-CD20 antibody is a humanized antibody.

In certain embodiments, the anti-CD20 antibody of an anti-CD20 antibody-cytotoxic agent conjugate of the invention comprises the variable region of C2B8, 1F5, FB1, 2H7, 93-1B3, 109-3C2, B1, B9E9, 7D1, H147, L26, L27, or MEM97. In certain more specific embodiments, such an anti-CD20 antibody is a chimeric antibody.

In certain embodiments, the anti-CD20 antibody of an anti-CD20 antibody-cytotoxic agent conjugate of the invention is an affinity maturated variant of C2B8, 1F5, FB1, 2H7, 93-1B3, 109-3C2, B1, B9E9, 7D1, H147, L26, L27, or MEM97.

In certain embodiments, the anti-CD20 antibody of an anti-CD20 antibody-cytotoxic agent conjugate is a bispecific antibody. In other embodiments, the anti-CD20 antibody is not a bispecific antibody.

The invention further provides a pharmaceutical composition comprising an anti-CD20 antibody-cytotoxic agent conjugate of the invention, for example any of the foregoing anti-CD20 antibody-cytotoxic agent conjugates. Exemplary anti-CD20 antibody-cytotoxic agent conjugates are described below.

The invention further provides a pharmaceutical composition comprising an anti-CD20 antibody-cytotoxic agent conjugate, wherein the cytotoxic agent of the anti-CD20 antibody-cytotoxic agent conjugate has an IC₅₀ of at least 40-fold less than the IC₅₀ of doxorubicin, and wherein the IC₅₀ of each of the cytotoxic agent and doxorubicin is measured by a method comprising: (a) culturing one or more CD20-expressing cell populations in the presence of one or more concentrations of the cytotoxic agent for a 72- to 96-hour period; (b) culturing one or more CD20-expressing cell populations in the presence of one or more concentrations of doxorubicin for a 72- to 96-hour period; and (c) identifying a concentration of the cytotoxic agent and doxorubicin, respectively, at which 50% fewer cells in the CD20-expressing cell populations of steps (a) and (b), respectively, are viable at the end of the period relative to a CD20-expressing cell population cultured in the absence of the cytotoxic agent and doxorubicin, wherein the CD20-expressing cell populations of steps (a), (b) and (c) are of the same cell type and are cultured under the same conditions, and wherein the concentration of the cytotoxic agent and doxorubicin identified in step (c) is the IC₅₀ of the cytotoxic agent and doxorubicin, respectively.

The invention further provides a pharmaceutical composition comprising an anti-CD20 antibody-cytotoxic agent conjugate, wherein the anti-CD20 antibody-cytotoxic agent conjugate has an IC₅₀ of at least 40-fold less than the IC₅₀ of an anti-CD20 antibody-doxorubicin conjugate, wherein the anti-CD20 antibody-cytotoxic agent conjugate and the anti-CD20 antibody-doxorubicin conjugate comprise the same anti-CD20 antibody, and wherein the IC₅₀ of each of the anti-CD20 antibody-cytotoxic agent conjugate and the anti-CD20 antibody-doxorubicin conjugate is measured by a method comprising: (a) culturing one or more CD20-expressing cell populations in the presence of one or more concentrations of the anti-CD20 antibody-cytotoxic agent conjugate for a 72- to 96-hour period; (b) culturing one or more CD20-expressing cell populations in the presence of one or more concentrations of the anti-CD20 antibody-doxorubicin conjugate for a 72- to 96-hour period; and (c) identifying a concentration of the anti-CD20 antibody-cytotoxic agent conjugate and the anti-CD20 antibody-doxorubicin conjugate, respectively, at which 50% fewer cells in the CD20-expressing cell populations of steps (a) and (b), respectively, are viable at the end of the period relative to a CD20-expressing cell population cultured in the absence of the anti-CD20 antibody-cytotoxic agent conjugate and the anti-CD20 antibody-doxorubicin conjugate, wherein the CD20-expressing cell populations of steps (a), (b) and (c) are of the same cell type and are cultured under the same conditions, and wherein the concentration of the anti-CD20 antibody-cytotoxic agent conjugate and the anti-CD20 antibody-doxorubicin conjugate identified in step (c) is the IC₅₀ of the anti-CD20 antibody-cytotoxic agent conjugate and the anti-CD20 antibody-doxorubicin conjugate, respectively.

The invention further provides, a pharmaceutical composition comprising an anti-CD20 antibody-cytotoxic agent conjugate, wherein the conjugate has a rate of accumulation in a CD20-expressing cell that is at least 20-fold greater than the rate of accumulation of an unconjugated form of the anti-CD20 antibody in the CD20-expressing cell, wherein the rates of accumulation of the conjugate and of the unconjugated form of the antibody are measured by a method comprising: (a) culturing a population of the CD20-expressing cell with the conjugate; (b) culturing a population of the CD20-expressing cell with the unconjugated antibody, wherein the populations of steps (a) and (b) are cultured under the same conditions; and (c) measuring the amount of the conjugate and unconjugated antibody accumulated in the populations of steps (a) and (b), respectively.

The invention further provides a pharmaceutical composition comprising an anti-CD20 antibody-cytotoxic agent conjugate, wherein the conjugate has a rate of accumulation in a CD20-expressing cell that is at least 20-fold greater than the rate of accumulation of an anti-CD20 antibody-doxorubicin conjugate in a CD20-expressing cell of the same cell type, wherein the anti-CD20 antibody-cytotoxic agent conjugate and the anti-CD20 antibody-doxorubicin conjugate comprise the same anti-CD20 antibody, and wherein the rates of accumulation of the anti-CD20 antibody-cytotoxic agent conjugate and of the anti-CD20 antibody-doxorubicin conjugate are measured by a method comprising: (a) culturing a population of the CD20-expressing cell with the anti-CD20 antibody-cytotoxic agent conjugate; (b) culturing a population of the CD20-expressing cell with the anti-CD20 antibody-doxorubicin conjugate, wherein the populations of steps (a) and (b) are cultured under the same conditions; and (c) measuring the amount of the anti-CD20 antibody-cytotoxic agent conjugate and anti-CD20 antibody-doxorubicin conjugate accumulated in the populations of steps (a) and (b), respectively.

The invention further provides a pharmaceutical composition comprising an anti-CD20 antibody-cytotoxic agent conjugate, wherein the conjugate exhibits an at least 1.5-fold greater accumulation in a non-peripheral region inside a CD20-expressing cell than the accumulation of an unconjugated form of the anti-CD20 antibody in the CD20-expressing cell, wherein the accumulation of the conjugate and of the unconjugated form of the antibody are measured by a method comprising: (a) culturing a population of the CD20-expressing cell with the conjugate; (b) culturing a population of the CD20-expressing cell with the unconjugated form of the anti-CD20 antibody; and (c) detecting by confocal fluorescence microscopy localization of the conjugate and the unconjugated form of the anti-CD20 antibody in the populations of steps (a) and (b), respectively, wherein the populations of steps (a) and (b) are cultured under the same conditions and for the same period of time, and wherein the conjugate exhibits an at least 1.5-fold greater accumulation in the CD20-expressing cell than the accumulation of the unconjugated form of the anti-CD20 antibody in the CD20-expressing cell if: (i) at least 1.5-fold as many cells of the population of step (a) contain a detectable amount of the conjugate in a non-peripheral region as the number of cells of the population of step (b) contain the unconjugated form of the antibody in a non-peripheral region; or (ii) the accumulation of the conjugate in a non-peripheral region of the majority of CD20-expressing cells of the population of step (a) is at least 1.5-fold greater than the accumulation of the unconjugated form of the anti-CD20 antibody in the majority of CD20-expressing cells of the population of step (b).

The invention further provides a pharmaceutical composition comprising an anti-CD20 antibody-cytotoxic agent conjugate, wherein the conjugate exhibits an at least 1.5-fold greater accumulation in a non-peripheral region inside a CD20-expressing cell than the accumulation of an anti-CD20 antibody-doxorubicin conjugate, wherein the anti-CD20 antibody-cytotoxic agent conjugate and the anti-CD20 antibody-doxorubicin conjugate comprise the same anti-CD20 antibody, in the CD20-expressing cell, wherein the accumulation of the conjugate and of the anti-CD20 antibody-doxorubicin conjugate are measured by a method comprising: (a) culturing a population of the CD20-expressing cell with the conjugate; (b) culturing a population of the CD20-expressing cell with the anti-CD20 antibody-doxorubicin conjugate; and (c) detecting by confocal fluorescence microscopy localization of the conjugate and the anti-CD20 antibody-doxorubicin conjugate in the populations of steps (a) and (b), respectively, wherein the populations of steps (a) and (b) are cultured under the same conditions and for the same period of time, and wherein the conjugate exhibits an at least 1.5-fold greater accumulation in the CD20-expressing cell than the accumulation of the anti-CD20 antibody-doxorubicin conjugate in the CD20-expressing cell if: (i) at least 1.5-fold as many cells of the population of step (a) contain a detectable amount of the conjugate in a non-peripheral region as the number of cells of the population of step (b) contain the anti-CD20 antibody-doxorubicin conjugate in a non-peripheral region; or (ii) the accumulation of the conjugate in a non-peripheral region of the majority of CD20-expressing cells of the population of step (a) is at least 1.5-fold greater than the accumulation of the anti-CD20 antibody-doxorubicin conjugate in the majority of CD20-expressing cells of the population of step (b).

The invention further provides a method of treating a CD20-expressing cancer, comprising administering to a subject in need of such treatment an effective amount of an anti-CD20 antibody-cytotoxic agent conjugate, wherein the cytotoxic agent of the anti-CD20 antibody-cytotoxic agent conjugate has an IC₅₀ of at least 40-fold less than the IC₅₀ of doxorubicin, and wherein the IC₅₀ of each of the cytotoxic agent and doxorubicin is measured by a method comprising: (a) culturing one or more CD20-expressing cell populations in the presence of one or more concentrations of the cytotoxic agent for a 72- to 96-hour period; (b) culturing one or more CD20-expressing cell populations in the presence of one or more concentrations of doxorubicin for a 72- to 96-hour period; and (c) identifying a concentration of the cytotoxic agent and doxorubicin, respectively, at which 50% fewer cells in the CD20-expressing cell populations of steps (a) and (b), respectively, are viable at the end of the period relative to a CD20-expressing cell population cultured in the absence of the cytotoxic agent and doxorubicin, wherein the CD20-expressing cell populations of steps (a), (b) and (c) are of the same cell type and are cultured under the same conditions, and wherein the concentration of the cytotoxic agent and doxorubicin identified in step (c) is the IC₅₀ of the cytotoxic agent and doxorubicin, respectively.

The invention further provides a method of treating a CD20-expressing cancer, comprising administering to a subject in need of such treatment an effective amount of an anti-CD20 antibody-cytotoxic agent conjugate, wherein the anti-CD20 antibody-cytotoxic agent conjugate has an IC₅₀ of at least 40-fold less than the IC₅₀ of an anti-CD20 antibody-doxorubicin conjugate, wherein the anti-CD20 antibody-cytotoxic agent conjugate and the anti-CD20 antibody-doxorubicin conjugate comprise the same anti-CD20 antibody, and wherein the IC₅₀ of each of the anti-CD20 antibody-cytotoxic agent conjugate and the anti-CD20 antibody-doxorubicin conjugate is measured by a method comprising: (a) culturing one or more CD20-expressing cell populations in the presence of one or more concentrations of the anti-CD20 antibody-cytotoxic agent conjugate for a 72- to 96-hour period; (b) culturing one or more CD20-expressing cell populations in the presence of one or more concentrations of the anti-CD20 antibody-doxorubicin conjugate for a 72- to 96-hour period; and (c) identifying a concentration of the anti-CD20 antibody-cytotoxic agent conjugate and the anti-CD20 antibody-doxorubicin conjugate, respectively, at which 50% fewer cells in the CD20-expressing cell populations of steps (a) and (b), respectively, are viable at the end of the period relative to a CD20-expressing cell population cultured in the absence of the anti-CD20 antibody-cytotoxic agent conjugate and the anti-CD20 antibody-doxorubicin conjugate, wherein the CD20-expressing cell populations of steps (a), (b) and (c) are of the same cell type and are cultured under the same conditions, and wherein the concentration of the anti-CD20 antibody-cytotoxic agent conjugate and the anti-CD20 antibody-doxorubicin conjugate identified in step (c) is the IC₅₀ of the anti-CD20 antibody-cytotoxic agent conjugate and the anti-CD20 antibody-doxorubicin conjugate, respectively.

The invention further provides a method of treating a CD20-expressing cancer, comprising administering to a subject in need of such treatment an effective amount of an anti-CD20 antibody-cytotoxic agent conjugate, wherein the conjugate has a rate of accumulation in a CD20-expressing cell that is at least 20-fold greater than the rate of accumulation of an unconjugated form of the anti-CD20 antibody in the CD20-expressing cell, wherein the rates of accumulation of the conjugate and of the unconjugated form of the antibody are measured by a method comprising: (a) culturing a population of the CD20-expressing cell with the conjugate; (b) culturing a population of the CD20-expressing cell with the unconjugated antibody, wherein the populations of steps (a) and (b) are cultured under the same conditions; and (c) measuring the amount of the conjugate and unconjugated antibody accumulated in the populations of steps (a) and (b), respectively.

The invention further provides a method of treating a CD20-expressing cancer, comprising administering to a subject in need of such treatment an effective amount of an anti-CD20 antibody-cytotoxic agent conjugate, wherein the conjugate has a rate of accumulation in a CD20-expressing cell that is at least 20-fold greater than the rate of accumulation of an anti-CD20 antibody-doxorubicin conjugate in a CD20-expressing cell of the same cell type, wherein the anti-CD20 antibody-cytotoxic agent conjugate and the anti-CD20 antibody-doxorubicin conjugate comprise the same anti-CD20 antibody, and wherein the rates of accumulation of the anti-CD20 antibody-cytotoxic agent conjugate and of the anti-CD20 antibody-doxorubicin conjugate are measured by a method comprising: (a) culturing a population of the CD20-expressing cell with the anti-CD20 antibody-cytotoxic agent conjugate; (b) culturing a population of the CD20-expressing cell with the anti-CD20 antibody-doxorubicin conjugate, wherein the populations of steps (a) and (b) are cultured under the same conditions; and (c) measuring the amount of the anti-CD20 antibody-cytotoxic agent conjugate and anti-CD20 antibody-doxorubicin conjugate accumulated in the populations of steps (a) and (b), respectively.

The invention further provides a method of treating a CD20-expressing cancer, comprising administering to a subject in need of such treatment an effective amount of an anti-CD20 antibody-cytotoxic agent conjugate, wherein the conjugate exhibits an at least 1.5-fold greater accumulation in a non-peripheral region inside a CD20-expressing cell than the accumulation of an unconjugated form of the anti-CD20 antibody in the CD20-expressing cell, wherein the accumulation of the conjugate and of the unconjugated form of the antibody are measured by a method comprising: (a) culturing a population of the CD20-expressing cell with the conjugate; (b) culturing a population of the CD20-expressing cell with the unconjugated form of the anti-CD20 antibody; and (c) detecting by confocal fluorescence microscopy localization of the conjugate and the unconjugated form of the anti-CD20 antibody in the populations of steps (a) and (b), respectively, wherein the populations of steps (a) and (b) are cultured under the same conditions and for the same period of time, and wherein the conjugate exhibits an at least 1.5-fold greater accumulation in the CD20-expressing cell than the accumulation of the unconjugated form of the anti-CD20 antibody in the CD20-expressing cell if: (i) at least 1.5-fold as many cells of the population of step (a) contain a detectable amount of the conjugate in a non-peripheral region as the number of cells of the population of step (b) contain the unconjugated form of the antibody in a non-peripheral region; or (ii) the accumulation of the conjugate in a non-peripheral region of the majority of CD20-expressing cells of the population of step (a) is at least 1.5-fold greater than the accumulation of the unconjugated form of the anti-CD20 antibody in the majority of CD20-expressing cells of the population of step (b).

The invention further provides a method of treating a CD20-expressing cancer, comprising administering to a subject in need of such treatment an effective amount of an anti-CD20 antibody-cytotoxic agent conjugate, wherein the conjugate exhibits an at least 1.5-fold greater accumulation in a non-peripheral region inside a CD20-expressing cell than the accumulation of an anti-CD20 antibody-doxorubicin conjugate, wherein the anti-CD20 antibody-cytotoxic agent conjugate and the anti-CD20 antibody-doxorubicin conjugate comprise the same anti-CD20 antibody, in the CD20-expressing cell, wherein the accumulation of the conjugate and of the anti-CD20 antibody-doxorubicin conjugate are measured by a method comprising: (a) culturing a population of the CD20-expressing cell with the conjugate; (b) culturing a population of the CD20-expressing cell with the anti-CD20 antibody-doxorubicin conjugate; and (c) detecting by confocal fluorescence microscopy localization of the conjugate and the anti-CD20 antibody-doxorubicin conjugate in the populations of steps (a) and (b), respectively, wherein the populations of steps (a) and (b) are cultured under the same conditions and for the same period of time, and wherein the conjugate exhibits an at least 1.5-fold greater accumulation in the CD20-expressing cell than the accumulation of the anti-CD20 antibody-doxorubicin conjugate in the CD20-expressing cell if: (i) at least 1.5-fold as many cells of the population of step (a) contain a detectable amount of the conjugate in a non-peripheral region as the number of cells of the population of step (b) contain the anti-CD20 antibody-doxorubicin conjugate in a non-peripheral region; or (ii) the accumulation of the conjugate in a non-peripheral region of the majority of CD20-expressing cells of the population of step (a) is at least 1.5-fold greater than the accumulation of the anti-CD20 antibody-doxorubicin conjugate in the majority of CD20-expressing cells of the population of step (b).

In certain embodiments, the cancer is a follicular Non-Hodgkin's Lymphoma, a small lymphocytic lymphoma, a chronic lymphocytic leukemia, a lymphoplasmacytic Non-Hodgkin's Lymphoma, a hairy cell leukemia, a B cell prolymphocytic leukemia, a CD20-positive Acute lymphocytic leukemia, or a marginal zone Non-Hodgkin's Lymphoma.

The invention further provides a method of treating an immune disorder involving CD20-expressing cells, comprising administering to a subject in need of such treatment an effective amount of an anti-CD20 antibody-cytotoxic agent conjugate, wherein the cytotoxic agent of the anti-CD20 antibody-cytotoxic agent conjugate has an IC₅₀ of at least 40-fold less than the IC₅₀ of doxorubicin, and wherein the IC₅₀ of each of the cytotoxic agent and doxorubicin is measured by a method comprising: (a) culturing one or more CD20-expressing cell populations in the presence of one or more concentrations of the cytotoxic agent for a 72- to 96-hour period; (b) culturing one or more CD20-expressing cell populations in the presence of one or more concentrations of doxorubicin for a 72- to 96-hour period; and (c) identifying a concentration of the cytotoxic agent and doxorubicin, respectively, at which 50% fewer cells in the CD20-expressing cell populations of steps (a) and (b), respectively, are viable at the end of the period relative to a CD20-expressing cell population cultured in the absence of the cytotoxic agent and doxorubicin, wherein the CD20-expressing cell populations of steps (a), (b) and (c) are of the same cell type and are cultured under the same conditions, and wherein the concentration of the cytotoxic agent and doxorubicin identified in step (c) is the IC₅₀ of the cytotoxic agent and doxorubicin, respectively.

The invention further provides method of treating an immune disorder involving CD20-expressing cells, comprising administering to a subject in need of such treatment an effective amount of an anti-CD20 antibody-cytotoxic agent conjugate, wherein the anti-CD20 antibody-cytotoxic agent conjugate has an IC₅₀ of at least 40-fold less than the IC₅₀ of an anti-CD20 antibody-doxorubicin conjugate, wherein the anti-CD20 antibody-cytotoxic agent conjugate and the anti-CD20 antibody-doxorubicin conjugate comprise the same anti-CD20 antibody, and wherein the IC₅₀ of each of the anti-CD20 antibody-cytotoxic agent conjugate and the anti-CD20 antibody-doxorubicin conjugate is measured by a method comprising: (a) culturing one or more CD20-expressing cell populations in the presence of one or more concentrations of the anti-CD20 antibody-cytotoxic agent conjugate for a 72- to 96-hour period; (b) culturing one or more CD20-expressing cell populations in the presence of one or more concentrations of the anti-CD20 antibody-doxorubicin conjugate for a 72- to 96-hour period; and (c) identifying a concentration of the anti-CD20 antibody-cytotoxic agent conjugate and the anti-CD20 antibody-doxorubicin conjugate, respectively, at which 50% fewer cells in the CD20-expressing cell populations of steps (a) and (b), respectively, are viable at the end of the period relative to a CD20-expressing cell population cultured in the absence of the anti-CD20 antibody-cytotoxic agent conjugate and the anti-CD20 antibody-doxorubicin conjugate, wherein the CD20-expressing cell populations of steps (a), (b) and (c) are of the same cell type and are cultured under the same conditions, and wherein the concentration of the anti-CD20 antibody-cytotoxic agent conjugate and the anti-CD20 antibody-doxorubicin conjugate identified in step (c) is the IC₅₀ of the anti-CD20 antibody-cytotoxic agent conjugate and the anti-CD20 antibody-doxorubicin conjugate, respectively.

The invention further provides a method of treating an immune disorder involving CD20-expressing cells, comprising administering to a subject in need of such treatment an effective amount of an anti-CD20 antibody-cytotoxic agent conjugate, wherein the conjugate has a rate of accumulation in a CD20-expressing cell that is at least 20-fold greater than the rate of accumulation of an unconjugated form of the anti-CD20 antibody in the CD20-expressing cell, and wherein the rates of accumulation of the conjugate and of the unconjugated form of the antibody are measured by a method comprising: (a) culturing a population of the CD20-expressing cell with the conjugate; (b) culturing a population of the CD20-expressing cell with the unconjugated antibody, wherein the populations of steps (a) and (b) are cultured under the same conditions; and (c) measuring the amount of the conjugate and unconjugated antibody accumulated in the populations of steps (a) and (b), respectively.

The invention further provides a method of treating an immune disorder involving CD20-expressing cells, comprising administering to a subject in need of such treatment an effective amount of an anti-CD20 antibody-cytotoxic agent conjugate, wherein the conjugate has a rate of accumulation in a CD20-expressing cell that is at least 20-fold greater than the rate of accumulation of an anti-CD20 antibody-doxorubicin conjugate in a CD20-expressing cell of the same cell type, wherein the anti-CD20 antibody-cytotoxic agent conjugate and the anti-CD20 antibody-doxorubicin conjugate comprise the same anti-CD20 antibody, and and wherein the rates of accumulation of the anti-CD20 antibody-cytotoxic agent conjugate and of the anti-CD20 antibody-doxorubicin conjugate are measured by a method comprising: (a) culturing a population of the CD20-expressing cell with the anti-CD20 antibody-cytotoxic agent conjugate; (b) culturing a population of the CD20-expressing cell with the anti-CD20 antibody-doxorubicin conjugate, wherein the populations of steps (a) and (b) are cultured under the same conditions; and (c) measuring the amount of the anti-CD20 antibody-cytotoxic agent conjugate and anti-CD20 antibody-doxorubicin conjugate accumulated in the populations of steps (a) and (b), respectively.

The invention further provides method of treating an immune disorder involving CD20-expressing cells, comprising administering to a subject in need of such treatment an effective amount of an anti-CD20 antibody-cytotoxic agent conjugate, wherein the conjugate exhibits an at least 1.5-fold greater accumulation in a non-peripheral region inside a CD20-expressing cell than the accumulation of an unconjugated form of the anti-CD20 antibody in the CD20-expressing cell, wherein the accumulation of the conjugate and of the unconjugated form of the antibody are measured by a method comprising: (a) culturing a population of the CD20-expressing cell with the conjugate; (b) culturing a population of the CD20-expressing cell with the unconjugated form of the anti-CD20 antibody; and (c) detecting by confocal fluorescence microscopy localization of the conjugate and the unconjugated form of the anti-CD20 antibody in the populations of steps (a) and (b), respectively, wherein the populations of steps (a) and (b) are cultured under the same conditions and for the same period of time, and wherein the conjugate exhibits an at least 1.5-fold greater accumulation in the CD20-expressing cell than the accumulation of the unconjugated form of the anti-CD20 antibody in the CD20-expressing cell if: (i) at least 1.5-fold as many cells of the population of step (a) contain a detectable amount of the conjugate in a non-peripheral region as the number of cells of the population of step (b) contain the unconjugated form of the antibody in a non-peripheral region; or (ii) the accumulation of the conjugate in a non-peripheral region of the majority of CD20-expressing cells of the population of step (a) is at least 1.5-fold greater than the accumulation of the unconjugated form of the anti-CD20 antibody in the majority of CD20-expressing cells of the population of step (b).

The invention further provides a method of treating an immune disorder involving CD20-expressing cells, comprising administering to a subject in need of such treatment an effective amount of an anti-CD20 antibody-cytotoxic agent conjugate, wherein the conjugate exhibits an at least 1.5-fold greater accumulation in a non-peripheral region inside a CD20-expressing cell than the accumulation of an anti-CD20 antibody-doxorubicin conjugate, wherein the anti-CD20 antibody-cytotoxic agent conjugate and the anti-CD20 antibody-doxorubicin conjugate comprise the same anti-CD20 antibody, in the CD20-expressing cell, wherein the accumulation of the conjugate and of the anti-CD20 antibody-doxorubicin conjugate are measured by a method comprising: (a) culturing a population of the CD20-expressing cell with the conjugate; (b) culturing a population of the CD20-expressing cell with the anti-CD20 antibody-doxorubicin conjugate; and (c) detecting by confocal fluorescence microscopy localization of the conjugate and the anti-CD20 antibody-doxorubicin conjugate in the populations of steps (a) and (b), respectively, wherein the populations of steps (a) and (b) are cultured under the same conditions and for the same period of time, and wherein the conjugate exhibits an at least 1.5-fold greater accumulation in the CD20-expressing cell than the accumulation of the anti-CD20 antibody-doxorubicin conjugate in the CD20-expressing cell if: (i) at least 1.5-fold as many cells of the population of step (a) contain a detectable amount of the conjugate in a non-peripheral region as the number of cells of the population of step (b) contain the anti-CD20 antibody-doxorubicin conjugate in a non-peripheral region; or (ii) the accumulation of the conjugate in a non-peripheral region of the majority of CD20-expressing cells of the population of step (a) is at least 1.5-fold greater than the accumulation of the anti-CD20 antibody-doxorubicin conjugate in the majority of CD20-expressing cells of the population of step (b).

In certain embodiments, the immune disorder is rheumatoid arthritis, multiple sclerosis, endocrine ophthalmopathy, uveoretinitis, systemic lupus erythematosus, myasthenia gravis, Grave's disease, glomerulonephritis, autoimmune hepatological disorder, autoimmune inflammatory bowel disease, anaphylaxis, allergic reaction, Sjogren's syndrome, juvenile onset (Type I) diabetes mellitus, primary biliary cirrhosis, Wegener's granulomatosis, fibromyalgia, inflammatory bowel disease, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, presenile dementia, demyelinating diseases, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyly, and telangiectasia), adult onset diabetes mellitus (Type II diabetes), male and female autoimmune infertility, ankylosing spondolytis, ulcerative colitis, Crohn's disease, mixed connective tissue disease, polyarteritis nedosa, systemic necrotizing vasculitis, juvenile onset rheumatoid arthritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic encephalomyelitis, toxic epidermal necrolysis, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Eaton-Lambert syndrome, relapsing polychondritis, cryoglobulinemia, Waldenstrom's macroglobulemia, Epstein-Barr virus infection, mumps, Evan's syndrome, non-cancerous lymphocytosis, pre-cancerous lymphocytosis, or autoimmune gonadal failure.

In certain embodiments, the immune disorder is rheumatoid arthritis, multiple sclerosis, endocrine ophthalmopathy, systemic lupus erythematosus, myasthenia gravis, Grave's disease, glomerulonephritis, anaphylaxis, allergic reaction, Sjogren's syndrome, juvenile onset (Type I) diabetes mellitus, primary biliary cirrhosis, Wegener's granulomatosis, inflammatory bowel disease, polymyositis, dermatomyositis, Schmidt's syndrome, Addison's disease, adrenalitis, thyroiditis, Hashimoto's thyroiditis, autoimmune thyroid disease, pernicious anemia, chronic hepatitis, lupoid hepatitis, atherosclerosis, demyelinating diseases, subacute cutaneous lupus erythematosus, hypoparathyroidism, autoimmune thrombocytopenia, idiopathic thrombocytopenic purpura, hemolytic anemia, pemphigus vulgaris, pemphigus, dermatitis herpetiformis, alopecia areata, pemphigoid, scleroderma, progressive systemic sclerosis, CREST syndrome (calcinosis, Raynaud's phenomenon, esophageal dysmotility, sclerodactyly, and telangiectasia), adult onset diabetes mellitus (Type II diabetes), ulcerative colitis, Crohn's disease, mixed connective tissue disease, polyarteritis nedosa, systemic necrotizing vasculitis, juvenile onset rheumatoid arthritis, atopic rhinitis, Goodpasture's syndrome, asthma, anti-phospholipid syndrome, farmer's lung, erythema multiforme, autoimmune chronic active hepatitis, bird-fancier's lung, allergic encephalomyelitis, toxic epidermal necrolysis, alveolitis, allergic alveolitis, fibrosing alveolitis, erythema nodosum, transfusion reaction, Caplan's syndrome, erythroblastosis fetalis, Felty's syndrome, IgA nephropathy, Henoch-Schonlein purpura, graft versus host disease, transplantation rejection, relapsing polychondritis, cryoglobulinemia, Waldenstrom's macroglobulemia, Epstein-Barr virus infection, non-cancerous lymphocytosis, pre-cancerous lymphocytosis, and autoimmune gonadal failure.

In certain embodiments, the methods of the invention for treating an immune disorder involving CD20-expressing cells further comprise administering to the subject an immunosuppressive agent. In certain specific embodiments, the immunosuppressive agent is cyclosporine, FK506, rapamycin, methotrexate, cyclophosphamide, or prednisone.

In certain embodiments, the methods of the invention for treating a CD20-expressing cancer and the methods for treating an immune disorder involving CD20-expressing cells further comprise administering to the subject a second cytostatic or cytotoxic agent. In certain embodiments, the method further comprises further administering to the subject a second antibody that binds to an antigen of the CD20-expressing cancer or the CD20-expressing cells, respectively, wherein the second antibody is not an anti-CD20 antibody. In certain specific embodiments, the second antibody is selected from the group consisting of an anti-CD19 antibody, an anti-CD22 antibody, an anti-CD30 antibody, and an anti-CD40 antibody. In certain more specific embodiments, the second antibody is conjugated to a second cytotoxic or cytostatic agent. The cytotoxic agent attached to the second antibody can be the same as the cytotoxic agent of the anti-CD20 antibody-cytotoxic agent conjugate of the invention, or it can be different. In certain specific embodiments, the second cytotoxic or cytostatic agent is a chemotherapeutic agent, a radioisotope, or a toxin. In certain embodiments, the subject is a mammal. In certain embodiments, the subject is human.

The present invention further provides kits comprising an anti-CD20 antibody-cytotoxic agent conjugate of the invention. Optionally, the kits may further comprise one or more additional therapeutic agents as described in Sections 5.12 and 5.13, for example an antibody or an immunosuppressive agent. Exemplary embodiments of the kits of the invention are described below.

For example, in certain embodiments, the present invention provides a kit comprising in a first container, an anti-CD20 antibody, and in a second container, a cytotoxic agent, wherein the cytotoxic agent has an IC₅₀ of at least 40-fold less than the IC₅₀ of doxorubicin, and wherein the IC₅₀ of each of the cytotoxic agent and doxorubicin is measured by a method comprising: (a) culturing one or more CD20-expressing cell populations in the presence of one or more concentrations of the cytotoxic agent for a 72- to 96-hour period; (b) culturing one or more CD20-expressing cell populations in the presence of one or more concentrations of doxorubicin for a 72- to 96-hour period; and (c) identifying a concentration of the cytotoxic agent and doxorubicin, respectively, at which 50% fewer cells in the CD20-expressing cell populations, respectively, are viable at the end of the period relative to a CD20-expressing cell population cultured in the absence of the cytotoxic agent and doxorubicin, wherein the CD20-expressing cell populations of steps (a), (b) and (c) are of the same cell type and are cultured under the same conditions, and wherein the concentration of the cytotoxic agent and doxorubicin identified in step (c) is the IC₅₀ of the cytotoxic agent and doxorubicin, respectively. In certain embodiments, the kit further comprises, in a third container, a linker for conjugating the anti-CD20 antibody to the cytotoxic agent.

In other embodiments, the invention further provides a kit comprising in a first container, an anti-CD20 antibody, and in a second container, a cytotoxic agent, wherein upon conjugation of the anti-CD20 antibody and the drug, the resulting conjugate has a rate of accumulation in a CD20-expressing cell that is at least 20-fold greater than the rate of accumulation of an unconjugated form of the anti-CD20 antibody in the CD20-expressing cell, and wherein the rates of accumulation of the conjugate and of the unconjugated form of the antibody are measured by a method comprising: (a) culturing a population of the CD20-expressing cell with the conjugate; (b) culturing a population of the CD20-expressing cell with the unconjugated antibody, wherein the populations of steps (a) and (b) are cultured under the same conditions; and (c) measuring the amount of the conjugate and unconjugated antibody accumulated in the populations of steps (a) and (b), respectively.

In yet other embodiments, the invention further provides a kit comprising in a first container, an anti-CD20 antibody, and in a second container, a cytotoxic agent, wherein upon conjugation of the anti-CD20 antibody and the drug, the resulting conjugate has a rate of accumulation in a CD20-expressing cell that is at least 20-fold greater than the rate of accumulation of an anti-CD20 antibody-doxorubicin conjugate in a CD20-expressing cell of the same cell type, wherein the anti-CD20 antibody-cytotoxic agent conjugate and the anti-CD20 antibody-doxorubicin conjugate comprise the same anti-CD20 antibody, and wherein the rates of accumulation of the anti-CD20 antibody-cytotoxic agent conjugate and of the anti-CD20 antibody-doxorubicin conjugate are measured by a method comprising: (a) culturing a population of the CD20-expressing cell with the anti-CD20 antibody-cytotoxic agent conjugate; (b) culturing a population of the CD20-expressing cell with the anti-CD20 antibody-doxorubicin conjugate, wherein the populations of steps (a) and (b) are cultured under the same conditions; and (c) measuring the amount of the anti-CD20 antibody-cytotoxic agent conjugate and anti-CD20 antibody-doxorubicin conjugate accumulated in the populations of steps (a) and (b), respectively.

In yet other embodiments, the invention further provides a kit comprising in a first container, an anti-CD20 antibody, and in a second container, a cytotoxic agent, wherein upon conjugation of the anti-CD20 antibody and the drug, the resulting conjugate exhibits an at least 1.5-fold greater accumulation in a non-peripheral region inside a CD20-expressing cell than the accumulation of an unconjugated form of the anti-CD20 antibody in the CD20-expressing cell, wherein the accumulation of the conjugate and of the unconjugated form of the antibody are measured by a method comprising: (a) culturing a population of the CD20-expressing cell with the conjugate; (b) culturing a population of the CD20-expressing cell with the unconjugated form of the anti-CD20 antibody; and (c) detecting by confocal fluorescence microscopy localization of the conjugate and the unconjugated form of the anti-CD20 antibody in the populations of steps (a) and (b), respectively, wherein the populations of steps (a) and (b) are cultured under the same conditions and for the same period of time, and wherein the conjugate exhibits an at least 1.5-fold greater accumulation in the CD20-expressing cell than the accumulation of the unconjugated form of the anti-CD20 antibody in the CD20-expressing cell if: (i) at least 1.5-fold as many cells of the population of step (a) contain a detectable amount of the conjugate in a non-peripheral region as the number of cells of the population of step (b) contain the unconjugated form of the antibody in a non-peripheral region; or (ii) the accumulation of the conjugate in a non-peripheral region of the majority of CD20-expressing cells of the population of step (a) is at least 1.5-fold greater than the accumulation of the unconjugated form of the anti-CD20 antibody in the majority of CD20-expressing cells of the population of step (b).

In yet other embodiments, the invention further provides a kit comprising in a first container, an anti-CD20 antibody, and in a second container, a cytotoxic agent, wherein upon conjugation of the anti-CD20 antibody and the drug, the resulting conjugate exhibits an at least 1.5-fold greater accumulation in a non-peripheral region inside a CD20-expressing cell than the accumulation of an anti-CD20 antibody-doxorubicin conjugate, wherein the anti-CD20 antibody-cytotoxic agent conjugate and the anti-CD20 antibody-doxorubicin conjugate comprise the same anti-CD20 antibody, in the CD20-expressing cell, wherein the accumulation of the conjugate and of the anti-CD20 antibody-doxorubicin conjugate are measured by a method comprising: (a) culturing a population of the CD20-expressing cell with the conjugate; (b) culturing a population of the CD20-expressing cell with the anti-CD20 antibody-doxorubicin conjugate; and (c) detecting by confocal fluorescence microscopy localization of the conjugate and the anti-CD20 antibody-doxorubicin conjugate in the populations of steps (a) and (b), respectively, wherein the populations of steps (a) and (b) are cultured under the same conditions and for the same period of time, and wherein the conjugate exhibits an at least 1.5-fold greater accumulation in the CD20-expressing cell than the accumulation of the anti-CD20 antibody-doxorubicin conjugate in the CD20-expressing cell if: (i) at least 1.5-fold as many cells of the population of step (a) contain a detectable amount of the conjugate in a non-peripheral region as the number of cells of the population of step (b) contain the anti-CD20 antibody-doxorubicin conjugate in a non-peripheral region; or (ii) the accumulation of the conjugate in a non-peripheral region of the majority of CD20-expressing cells of the population of step (a) is at least 1.5-fold greater than the accumulation of the anti-CD20 antibody-doxorubicin conjugate in the majority of CD20-expressing cells of the population of step (b).

In yet other embodiments, the invention further provides a kit comprising in a first container, an anti-CD20 antibody, in a second container, a cytotoxic agent, and in a third container, a linker for conjugating the anti-CD20 antibody to the cytotoxic agent, wherein upon conjugation of the anti-CD20 antibody and the drug via the linker, the resulting conjugate has a rate of accumulation in a CD20-expressing cell that is at least 20-fold greater than the rate of accumulation of an unconjugated form of the anti-CD20 antibody in the CD20-expressing cell, and wherein the rates of accumulation of the conjugate and of the unconjugated form of the antibody are measured by a method comprising: (a) culturing a population of the CD20-expressing cell with the conjugate; (b) culturing a population of the CD20-expressing cell with the unconjugated antibody, wherein the populations of steps (a) and (b) are cultured under the same conditions; and (c) measuring the amount of the conjugate and unconjugated antibody accumulated in the populations of steps (a) and (b), respectively.

In yet other embodiments, the invention further provides a kit comprising in a first container, an anti-CD20 antibody, in a second container, a cytotoxic agent, and in a third container, a linker for conjugating the anti-CD20 antibody to the cytotoxic agent, wherein upon conjugation of the anti-CD20 antibody and the drug via the linker, the resulting conjugate exhibits an at least 1.5-fold greater accumulation in a non-peripheral region inside a CD20-expressing cell than the accumulation of an unconjugated form of the anti-CD20 antibody in the CD20-expressing cell, wherein the accumulation of the conjugate and of the unconjugated form of the antibody are measured by a method comprising: (a) culturing a population of the CD20-expressing cell with the conjugate; (b) culturing a population of the CD20-expressing cell with the unconjugated form of the anti-CD20 antibody; and (c) detecting by confocal fluorescence microscopy localization of the conjugate and the unconjugated form of the anti-CD20 antibody in the populations of steps (a) and (b), respectively, wherein the populations of steps (a) and (b) are cultured under the same conditions and for the same period of time, and wherein the conjugate exhibits an at least 1.5-fold greater accumulation in the CD20-expressing cell than the accumulation of the unconjugated form of the anti-CD20 antibody in the CD20-expressing cell if: (i) at least 1.5-fold as many cells of the population of step (a) contain a detectable amount of the conjugate in a non-peripheral region as the number of cells of the population of step (b) contain the unconjugated form of the antibody in a non-peripheral region; or (ii) the accumulation of the conjugate in a non-peripheral region of the majority of CD20-expressing cells of the population of step (a) is at least 1.5-fold greater than the accumulation of the unconjugated form of the anti-CD20 antibody in the majority of CD20-expressing cells of the population of step (b).

In yet other embodiments, the invention further provides a kit comprising in a first container, an anti-CD20 antibody, in a second container, a cytotoxic agent, and in a third container, a linker for conjugating the anti-CD20 antibody to the cytotoxic agent, wherein upon conjugation of the anti-CD20 antibody and the drug via the linker, the resulting conjugate exhibits an at least 1.5-fold greater accumulation in a non-peripheral region inside a CD20-expressing cell than the accumulation of an anti-CD20 antibody-doxorubicin conjugate, wherein the anti-CD20 antibody-cytotoxic agent conjugate and the anti-CD20 antibody-doxorubicin conjugate comprise the same anti-CD20 antibody, in the CD20-expressing cell, wherein the accumulation of the conjugate and of the anti-CD20 antibody-doxorubicin conjugate are measured by a method comprising: (a) culturing a population of the CD20-expressing cell with the conjugate; (b) culturing a population of the CD20-expressing cell with the anti-CD20 antibody-doxorubicin conjugate; and (c) detecting by confocal fluorescence microscopy localization of the conjugate and the anti-CD20 antibody-doxorubicin conjugate in the populations of steps (a) and (b), respectively, wherein the populations of steps (a) and (b) are cultured under the same conditions and for the same period of time, and wherein the conjugate exhibits an at least 1.5-fold greater accumulation in the CD20-expressing cell than the accumulation of the anti-CD20 antibody-doxorubicin conjugate in the CD20-expressing cell if: (i) at least 1.5-fold as many cells of the population of step (a) contain a detectable amount of the conjugate in a non-peripheral region as the number of cells of the population of step (b) contain the anti-CD20 antibody-doxorubicin conjugate in a non-peripheral region; or (ii) the accumulation of the conjugate in a non-peripheral region of the majority of CD20-expressing cells of the population of step (a) is at least 1.5-fold greater than the accumulation of the anti-CD20 antibody-doxorubicin conjugate in the majority of CD20-expressing cells of the population of step (b).

In yet other embodiments, the invention further provides a kit comprising in a first container, an anti-CD20 antibody, in a second container, a cytotoxic agent, and in a third container, a linker for conjugating the anti-CD20 antibody to the cytotoxic agent, wherein upon conjugation of the anti-CD20 antibody and the drug via the linker, the resulting conjugate has a rate of accumulation in a CD20-expressing cell that is at least 20-fold greater than the rate of accumulation of an anti-CD20 antibody-doxorubicin conjugate in a CD20-expressing cell of the same cell type, wherein the anti-CD20 antibody-cytotoxic agent conjugate and the anti-CD20 antibody-doxorubicin conjugate comprise the same anti-CD20 antibody, and wherein the rates of accumulation of the anti-CD20 antibody-cytotoxic agent conjugate and of the anti-CD20 antibody-doxorubicin conjugate are measured by a method comprising: (a) culturing a population of the CD20-expressing cell with the anti-CD20 antibody-cytotoxic agent conjugate; (b) culturing a population of the CD20-expressing cell with the anti-CD20 antibody-doxorubicin conjugate, wherein the populations of steps (a) and (b) are cultured under the same conditions; and (c) measuring the amount of the anti-CD20 antibody-cytotoxic agent conjugate and anti-CD20 antibody-doxorubicin conjugate accumulated in the populations of steps (a) and (b), respectively.

In yet other embodiments, the invention further provides a kit comprising: (a) an anti-CD20 antibody-cytotoxic agent conjugate, wherein the cytotoxic agent of the anti-CD20 antibody-cytotoxic agent conjugate has an IC₅₀ of at least 40-fold less than the IC₅₀ of doxorubicin, and wherein the IC₅₀ of each of the cytotoxic agent and doxorubicin is measured by a method comprising: (i) culturing one or more CD20-expressing cell populations in the presence of one or more concentrations of the cytotoxic agent for a 72- to 96-hour period; (ii) culturing one or more CD20-expressing cell populations in the presence of one or more concentrations of doxorubicin for a 72- to 96-hour period; and (iii) identifying a concentration of the cytotoxic agent and doxorubicin, respectively, at which 50% fewer cells in the CD20-expressing cell populations of steps (i) and (ii), respectively, are viable at the end of the period relative to a CD20-expressing cell population cultured in the absence of the cytotoxic agent and doxorubicin, wherein the CD20-expressing cell populations of steps (i), (ii) and (iii) are of the same cell type and are cultured under the same conditions, and wherein the concentration of the cytotoxic agent and doxorubicin identified in step (iii) is the IC₅₀ of the cytotoxic agent and doxorubicin, respectively, and (b) a notice by a regulatory agency indicating approval for manufacture, use or sale of the conjugate for human administration.

In yet other embodiments, the invention further provides a kit comprising: (a) an anti-CD20 antibody-cytotoxic agent conjugate, wherein the anti-CD20 antibody-cytotoxic agent conjugate has an IC₅₀ of at least 40-fold less than the IC₅₀ of an anti-CD20 antibody-doxorubicin conjugate, wherein the anti-CD20 antibody-cytotoxic agent conjugate and the anti-CD20 antibody-doxorubicin conjugate comprise the same anti-CD20 antibody, and wherein the IC₅₀ of each of the anti-CD20 antibody-cytotoxic agent conjugate and the anti-CD20 antibody-doxorubicin conjugate is measured by a method comprising: (i) culturing one or more CD20-expressing cell populations in the presence of one or more concentrations of the anti-CD20 antibody-cytotoxic agent conjugate for a 72- to 96-hour period; (ii) culturing one or more CD20-expressing cell populations in the presence of one or more concentrations of the anti-CD20 antibody-doxorubicin conjugate for a 72- to 96-hour period; and (iii) identifying a concentration of the anti-CD20 antibody-cytotoxic agent conjugate and the anti-CD20 antibody-doxorubicin conjugate, respectively, at which 50% fewer cells in the CD20-expressing cell populations of steps (i) and (ii), respectively, are viable at the end of the period relative to a CD20-expressing cell population cultured in the absence of the anti-CD20 antibody-cytotoxic agent conjugate and the anti-CD20 antibody-doxorubicin conjugate, wherein the CD20-expressing cell populations of steps (i), (ii) and (iii) are of the same cell type and are cultured under the same conditions, and wherein the concentration of the anti-CD20 antibody-cytotoxic agent conjugate and the anti-CD20 antibody-doxorubicin conjugate identified in step (iii) is the IC₅₀ of the anti-CD20 antibody-cytotoxic agent conjugate and the anti-CD20 antibody-doxorubicin conjugate, respectively; and (b) a notice by a regulatory agency indicating approval for manufacture, use or sale of the conjugate for human administration.

In yet other embodiments, the invention further provides a kit comprising: (a) an anti-CD20 antibody-cytotoxic agent conjugate, wherein the conjugate has a rate of accumulation in a CD20-expressing cell that is at least 20-fold greater than the rate of accumulation of an unconjugated form of the anti-CD20 antibody in the CD20-expressing cell, and wherein the rates of accumulation of the conjugate and of the unconjugated form of the antibody are measured by a method comprising: (i) culturing a population of the CD20-expressing cell with the conjugate; (ii) culturing a population of the CD20-expressing cell with the unconjugated antibody, wherein the populations of steps (i) and (ii) are cultured under the same conditions; and (iii) measuring the amount of the conjugate and unconjugated antibody accumulated in the populations of steps (i) and (ii), respectively; and (b) a notice by a regulatory agency indicating approval for manufacture, use or sale of the conjugate for human administration.

In yet other embodiments, the invention further provides a kit comprising: (a) an anti-CD20 antibody-cytotoxic agent conjugate, wherein the conjugate has a rate of accumulation in a CD20-expressing cell that is at least 20-fold greater than the rate of accumulation of an anti-CD20 antibody-doxorubicin conjugate in a CD20-expressing cell of the same cell type, wherein the anti-CD20 antibody-cytotoxic agent conjugate and the anti-CD20 antibody-doxorubicin conjugate comprise the same anti-CD20 antibody, and wherein the rates of accumulation of the anti-CD20 antibody-cytotoxic agent conjugate and of the anti-CD20 antibody-doxorubicin conjugate are measured by a method comprising: (i) culturing a population of the CD20-expressing cell with the anti-CD20 antibody-cytotoxic agent conjugate; (ii) culturing a population of the CD20-expressing cell with the anti-CD20 antibody-doxorubicin conjugate, wherein the populations of steps (i) and (ii) are cultured under the same conditions; and (iii) measuring the amount of the anti-CD20 antibody-cytotoxic agent conjugate and anti-CD20 antibody-doxorubicin conjugate accumulated in the populations of steps (i) and (ii), respectively; and (b) a notice by a regulatory agency indicating approval for manufacture, use or sale of the conjugate for human administration.

In yet other embodiments, the invention further provides a kit comprising: (a) an anti-CD20 antibody-cytotoxic agent conjugate, wherein the conjugate exhibits an at least 1.5-fold greater accumulation in a non-peripheral region inside a CD20-expressing cell than the accumulation of an unconjugated form of the anti-CD20 antibody in the CD20-expressing cell, wherein the accumulation of the conjugate and of the unconjugated form of the antibody are measured by a method comprising: (i) culturing a population of the CD20-expressing cell with the conjugate; (ii) culturing a population of the CD20-expressing cell with the unconjugated form of the anti-CD20 antibody; and (iii) detecting by confocal fluorescence microscopy localization of the conjugate and the unconjugated form of the anti-CD20 antibody in the populations of steps (a) and (b), respectively, wherein the populations of steps (a) and (b) are cultured under the same conditions and for the same period of time, and wherein the conjugate exhibits an at least 1.5-fold greater accumulation in the CD20-expressing cell than the accumulation of the unconjugated form of the anti-CD20 antibody in the CD20-expressing cell if: (A) at least 1.5-fold as many cells of the population of step (a) contain a detectable amount of the conjugate in a non-peripheral region as the number of cells of the population of step (b) contain the unconjugated form of the antibody in a non-peripheral region; or (B) the accumulation of the conjugate in a non-peripheral region of the majority of CD20-expressing cells of the population of step (a) is at least 1.5-fold greater than the accumulation of the unconjugated form of the anti-CD20 antibody in the majority of CD20-expressing cells of the population of step (ii); and (b) a notice by a regulatory agency indicating approval for manufacture, use or sale of the conjugate for human administration.

In yet other embodiments, the invention further provides a kit comprising: (a) an anti-CD20 antibody-cytotoxic agent conjugate, wherein the conjugate exhibits an at least 1.5-fold greater accumulation in a non-peripheral region inside a CD20-expressing cell than the accumulation of an anti-CD20 antibody-doxorubicin conjugate, wherein the anti-CD20 antibody-cytotoxic agent conjugate and the anti-CD20 antibody-doxorubicin conjugate comprise the same anti-CD20 antibody, in the CD20-expressing cell, wherein the accumulation of the conjugate and of the anti-CD20 antibody-doxorubicin conjugate are measured by a method comprising: (i) culturing a population of the CD20-expressing cell with the conjugate; (ii) culturing a population of the CD20-expressing cell with the anti-CD20 antibody-doxorubicin conjugate; and (iii) detecting by confocal fluorescence microscopy localization of the conjugate and the anti-CD20 antibody-doxorubicin conjugate in the populations of steps (a) and (b), respectively, wherein the populations of steps (a) and (b) are cultured under the same conditions and for the same period of time, and wherein the conjugate exhibits an at least 1.5-fold greater accumulation in the CD20-expressing cell than the accumulation of the anti-CD20 antibody-doxorubicin conjugate in the CD20-expressing cell if: (A) at least 1.5-fold as many cells of the population of step (a) contain a detectable amount of the conjugate in a non-peripheral region as the number of cells of the population of step (b) contain the anti-CD20 antibody-doxorubicin conjugate in a non-peripheral region; or (B) the accumulation of the conjugate in a non-peripheral region of the majority of CD20-expressing cells of the population of step (a) is at least 1.5-fold greater than the accumulation of the anti-CD20 antibody-doxorubicin conjugate in the majority of CD20-expressing cells of the population of step (ii); and (b) a notice by a regulatory agency indicating approval for manufacture, use or sale of the conjugate for human administration.

The invention further provides an anti-CD20 antibody-cytotoxic agent conjugate, wherein the conjugate is purified.

The invention further provides a pharmaceutical composition comprising an anti-CD20 antibody-cytotoxic agent conjugate, wherein the anti-CD20 antibody-cytotoxic agent conjugate is purified.

The invention further provides a method comprising administering an anti-CD20 antibody-cytotoxic agent conjugate, wherein the anti-CD20 antibody-cytotoxic agent conjugate is purified.

The invention further provides a kit comprising an anti-CD20 antibody-cytotoxic agent conjugate, wherein the anti-CD20 antibody-cytotoxic agent conjugate is purified.

In a preferred embodiment, the cytotoxic agent is not a radioisotope. In another preferred embodiment, the cytotoxic agent is not a toxin. In a more specific preferred embodiment, the cytotoxic agent is not ricin.

In certain embodiments, the kit further comprises a second cytotoxic or a cytostatic agent. In certain more specific embodiments, the second cytotoxic or cytostatic agent is selected from the group consisting of an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, a vinca alkaloid, a purine antagonist, and a dihydrofolate reductase inhibitor. In certain embodiments, the second cytotoxic or cytostatic agent is androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16, VM-26, azothioprine, mycophenolate mofetil, methotrexate, acyclovir, gangcyclovir, zidovudine, vidarabine, ribavarin, azidothymidine, cytidine arabinoside, amantadine, dideoxyuridine, iododeoxyuridine, poscarnet, or trifluridine.

In certain embodiments, the kit further comprises a second antibody other than an anti-CD20 antibody. In certain embodiments, the second antibody is an anti-CD19 antibody, an anti-CD22 antibody, an anti-CD30 antibody, and an anti-CD40 antibody. In certain embodiments, the second antibody is conjugated to a second cytotoxic or cytostatic agent.

### 4. BRIEF DESCRIPTION OF THE FIGURES

FIG. 1. Synthesis and structures of linker-drug systems used for mAb conjugation. (a) Synthesis of vcMMAE from the MMAE and vc-containing linker; (b) Synthesis of vcDox from doxorubicin and the vc-containing linker. Conjugates were prepared by reduction of internal mAb disulfides with dithiothreitol, followed by addition of the linker-drugs shown above. Stable thioether-linked ADCs were formed upon addition of the free sulfhydryl groups on the mAbs to the maleimides present on the drugs. The ADCs contained approximately 6-7 drugs/mAb.

FIG. 2. Cell characterization for CD20. The human B cell lymphoma lines Daudi, Raji and Ramos and the anaplastic large cell lymphoma line, Karpas, were evaluated by flow cytometry to assess their relative expression levels of CD20. Shown are the resultant fluorescent intensity profiles for unstained cells, those stained with the detecting secondary reagent alone and those stained with anti-CD20 mAb followed by a secondary goat anti-human IgG-FITC reagent.

FIG. 3. Binding comparison of anti-CD20 mAbs and ADC. Increasing concentrations of either Rituximab, mAb 1F5 or their respective ADCs were combined with Daudi cells, incubated on ice to block antigen modulation and washed. The bound mAb or ADC was then detected by excess goat anti-human or goat anti-mouse IgG-FITC as described in Materials and Methods.

FIG. 4. Sensitivity of Cells to ADCs. Cells in complete media were incubated with titrations of mAb and ADCs for 2 h to allow binding, washed to remove unbound reagent, replated in fresh complete media and returned to incubation for an additional 94 h. Alamar blue was added to culture wells 4 h prior to harvest. Cell viability assessed by detecting dye metabolism on a fluorescent plate reader as described in Materials and Methods and compared to that of control wells cultured in the presence of complete media alone. Dose response to these agents is shown for CD20-positive Raji cells (A), and Ramos cells (B), and CD20 negative Karpas cells (C). Points are an average of quadruplicate determinations.

FIG. 5. Selective induction of Apoptosis. Ramos cells were incubated with 5 μg/ml of Rituximab, Rituximab-vcMMAE, Rituximab-vcDOX, or medium alone. At the designated times cells were removed from cultures and stained with Annexin V-FITC and propidium iodide (PI) as described in Materials and Methods. The range of apoptotic cells (Annexin V⁺/PI⁻) and of dead cells (Annexin V⁺/PI⁺) was determined by flow cytometric analysis of each population.

FIG. 6. Cellular localization of Rituximab and Rituximab-ADC. Ramos B cells were treated with Rituximab, Rituximab-vcMMAE, or Rituximab-vcDOX in complete media at 37° C., and at the indicated times were collected, fixed and permeabilized by paraformaldehyde/saponin (Cytofix/Cytoperm™ Buffer, BD PharMingen, San Diego, Calif.). After blocking with goat IgG, cells were stained with a goat anti-human IgG Fc_-specific FITC conjugate the localization of fluorescence signals was then examined by Deltavision confocal microscopy. Shown are representative, composite (pile-up) images and single images from the respective composite captured through the equator or cell midpoint at 24 h post-treatment for Rituximab, and at progressive time points for Rituximab-vcMMAE or Rituximab-vcDOX. No staining was observed in parallel studies done with CD20⁻ Karpas cells (data not shown).

FIG. 7. Efficacy of Rituximab-ADC in an NHL model. (A) Antitumor activity of Rituximab and Rituximab ADCs were evaluated in subcutaneous Ramos NHL tumor model in SCID mice. Mice were implanted with 5×10⁶ L540cy Hodgkin's disease cells into the right flank. Groups of mice (five/group) either were left untreated or received Rituximab, Rituximab-vcMMAE, Rituximab-vcDox or an irrelevant ADC, BR96-vcMMAE on a schedule of q4dx3 starting when the tumor size in each group of 5 animals averaged 100 mm³.

### 5. DETAILED DESCRIPTION OF THE INVENTION

Anti-CD20 mAb conjugates were previously shown to be ineffective when linked with the anti-cancer drug doxorubicin (Braslawsky et al., 1991,Cancer Immunol Immunother. 33:367-74) or with toxins (Goulet et al., 1997, Blood 90(6):2364-75) suggesting that CD20 does not constitute a viable target for mAb-mediated drug delivery to the inside of cells. CD20 is displayed at variable but reasonably high levels on the surface of malignant B cells (from 2×10⁴-4×10⁵/cell; Vervoordeldonk et al., 1994, Cancer 73(3 Suppl): 1006-11; and herein) and is internalized and redistributed by the process of receptor-mediated endocytosis (Pulczynski et al., 1994, Leuk Res. 18:541-52). Various combinations of cell lines and mAbs have lead to differing reports of rates of CD20 receptor modulation in the presence or absence of reactive mAbs (Pulczynski et al., 1994, Leuk Res. 18:541-52; Vervoordeldonk et al., 1994, Cancer 73(3 Suppl):1006-11). Taken together, the data indicate that CD20 either does not internalize or weakly internalizes. Based on these findings researchers have focused on using approaches to CD20-directed mAb therapy that did not involve delivery of payloads to the inside of cells. Most notable has been the use of mAbs alone that can initiate tumor cell killing though signal transduction and complement-mediated mechanisms, and through Antibody-dependent cell-mediated cytotoxicity (ADCC), such as Rituximab.

In contrast to the previous failed attempts to generate ADCs and immunotoxins comprising CD20, the present inventors have identified an effective system for delivery of cytotoxic agents using anti-CD20 antibodies. Although this system is exemplified (in Section 6, infra) with Rituximab-vcMMAE ADC and 1F5-vcMMAE ADCs (i.e., ADCs containing the anti-CD20 antibodies Rituximab and 1F5, respectively, linked to the drug monomethyl Auristatin E through a vc linker), the system can be used with a variety of other anti-CD20 antibodies, drugs, and linkers as described in the following sections.

Accordingly, the present invention provides anti-CD20 ADCs comprising anti-CD20 antibodies (described in Sections 5.1-5.3, infra) conjugated to cytotoxic agents (described in Section 5.4), particularly those that have a high potency (see Section 5.4.1) and/or is capable of promoting net accumulation of the anti-CD20 ADC into CD20-expressing cells (see Section 5.4.2), for example by way of enhancing cellular uptake of the ADC relative to an unconjugated form of the antibody. The antibody unit of an ADC of the invention is preferably conjugated to the cytotoxic agent of the ADC via a linker, most preferably a linker that is hydrolyzed upon uptake of the ADC into a CD20-expressing cell (see Section 5.1). The present invention yet further provides methods of treatment (see Section 5.5) of CD20-expressing cancers (see Section 5.8) and immune disorders (see Section 5.9) involving CD20-expressing cells, comprising administering to a patient in need of such treatment an anti-CD20 ADC of the invention, in either single therapy or combination therapy (see Sections 5.12 and 5.13) regimens. The present invention further provides pharmaceutical compositions (see Section 5.6) and kits (see Section 5.11) comprising such conjugates.

### 5.1 Anti-CD20 Antibodies

The present invention encompasses anti-CD20 antibody-drug conjugates and their use to treat CD20-expressing cancers, for example cancers of B cell origin, and immune disorders mediated by or involving CD20-expressing cells.

Any human, humanized or chimeric anti-CD20 antibody can be employed in the methods and compositions of the invention. In a highly preferred embodiment, the anti-CD20 antibody comprises the variable region or the CDRs of monoclonal antibody 2B8. In a preferred mode of the embodiment, the anti-CD20 antibody is chimeric 2B8 antibody (“C2B8”).

C2B8 is commercially available as Rituximab, and has been approved by the FDA for the treatment of patients with relapsed or refractory, low grade or follicular, CD20-positive, B-cell non-Hodgkin's lymphoma (NHL).

In yet another mode of the embodiment, the C2B8 antibody is glycosylated with bisected oligosaccharides (Jean-Mairet et al., 2000, abstract no. 698; International Society for Preventive Oncology Meeting 2000).

In other embodiments, the anti-CD20 antibody comprises the variable region or the CDRs of one or more of the following anti-CD20 monoclonal antibodies: 1F5, FB1, 2H7, 93-1B3, 109-3C2, B1, B9E9, 7D1, H147, L26, L27, and MEM-97.

Many of these anti-CD20 antibodies have been administered to humans in clinical trials and been deemed safe for human use. For example, radio-iodinated B1 has been used in the treatment of B-cell lymphoma (see, e.g., Kaminski et al., 1993, New Eng. J. Med. 329(7):459-465) and has been accepted for fast-track FDA approval under the trademark BEXXAR (see, e.g., The Scientist 14[4]:16, Feb. 21, 2000). FB1 was described by Nozawa et al., 1999, Fukushima J. Med. Sci. 45:1-11.

Many of the anti-CD20 antibodies are available commercially, either as the purified monoclonal antibody or the antibody-secreting hybridoma. Sources of the anti-CD20 antibodies or hybridomas include Lab Vision Corporation, Fremont, Calif. (93-1B3 antibody); Bioprobe BV, the Netherlands (B9E9, 93-1B3 and 109-3C2 hybridomas); Serotec, United Kingdom (2H7, 7D1 and H147 antibodies); ID Labs, Ontario, Canada (2H7 antibody); Ancell, Bayport, Minn. (2H7 antibody); the American Type Culture Collection, Manassas, Va. (chimeric 2H7-expressing cell line C273; 1F5 hybridoma).

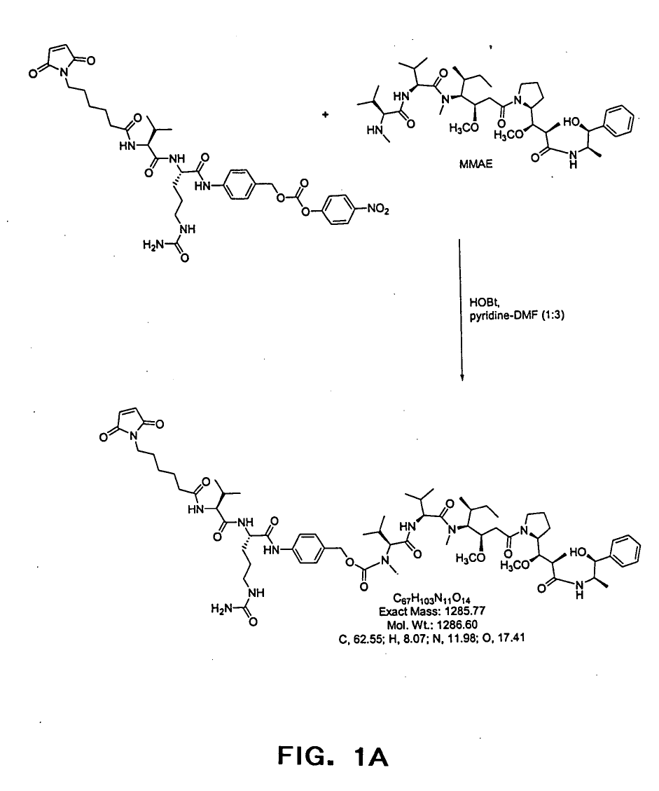

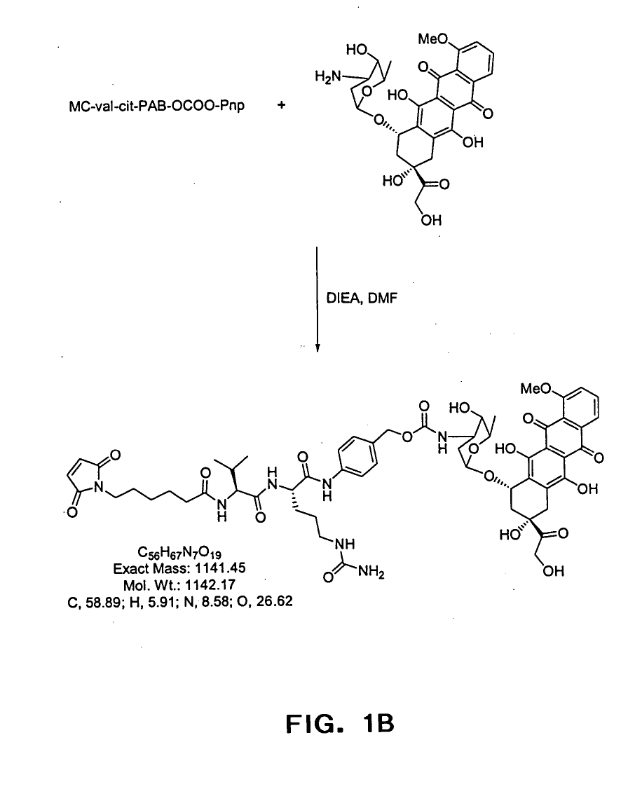

Further, the sequence of some of the anti-CD20 antibodies is known, MEM-97 has been partially sequenced (Dubel et al., 1994, J. Immunol. Methods 175:89-95). A comparison of the variable regions of the light and heavy chains of the anti-CD20 antibodies C2B8, B9E9, and 1F5 is provided in FIG. 1 of Schultz et al., 2000, Cancer Research 60:6663-6669.

The anti-CD20 antibodies used in the present methods and compositions are preferably monoclonal, and may be multispecific, human, humanized or chimeric antibodies, single chain antibodies, Fab fragments, F(ab′) fragments, fragments produced by a Fab expression library, and CD20 binding fragments of any of the above. The term “antibody,” as used herein, refers to immunoglobulin molecules and immunologically active portions of immunoglobulin molecules, i.e., molecules that contain an antigen binding site that immunospecifically binds CD20. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the invention, CD20-human antigen-binding antibody fragments can be used in the present invention include, but are not limited to, Fab, Fab′ and F(ab′)₂, Fd, single-chain Fvs (scFv), single-chain antibodies, disulfide-linked Fvs (sdfv) and fragments comprising either a V_{L} or V_{H} domain. Antigen-binding antibody fragments, including single-chain antibodies, may comprise the CD20-binding variable region(s) alone or in combination with the entirety or a portion of the following: hinge region, CH1, CH2, CH3 and CL domains. Also included in the invention are antigen-binding fragments also comprising any combination of variable region(s) with a hinge region, CH1, CH2, CH3 and CL domains. Preferably, the variable regions are derived human, murine (e.g., mouse and rat), donkey, sheep, rabbit, goat, guinea pig, camelid, horse, or chicken antibodies. As used herein, “human” antibodies include antibodies having the amino acid sequence of a human immunoglobulin and include antibodies isolated from human immunoglobulin libraries, from human B-cells, or from animals transgenic for one or more human immunoglobulin, as described infra and, for example in U.S. Pat. No. 5,939,598 by Kucherlapati et al.

The anti-CD20 antibodies that may be used in the methods of the present invention may be monospecific, bispecific, trispecific or of greater multispecificity. Multispecific antibodies may be specific for different epitopes of CD20 or may be specific for both CD20 as well as for a heterologous protein. See, e.g., PCT publications WO 93/17715; WO 92/08802; WO 91/00360; WO 92/05793; Tutt, et al., 1991, J. Immunol. 147:60-69; U.S. Pat. Nos. 4,474,893; 4,714,681; 4,925,648; 5,573,920; 5,601,819; Kostelny et al., 1992, J. Immunol. 148:1547-1553.

Antibodies of the present invention may be described or specified in terms of the particular variable regions or CDRs they comprise. In certain embodiments antibodies of the invention comprise one or more CDRs of the anti-CD20 antibodies 2B8, FB1, 1F5, 2H7, 93-1B3, 109-3C2, B1, B9E9, 7D1, H147, L26, L27, and MEM-97. In a preferred embodiment, those antibodies comprise human constant regions. In a most preferred embodiment, those antibodies comprise human constant and framework regions. Methods of generating such antibodies are described below.

Additionally, anti-CD20 antibodies for use in the methods and compositions of the present invention may also be described or specified in terms of their primary structures. Antibodies having regions of at least 50%, at least 55%, at least 60%, at least 65%, at least 70%, at least 75%, at least 80%, at least 85%, at least 90%, at least 95% and most preferably at least 98% identity (as calculated using methods known in the art and described herein in Section 5.1.1) to the CDRs or variable regions of 2B8, FB1, 1F5, 2H7, 93-1B3, 109-3C2, B1, B9E9, 7D1, H147, L26, L27, and MEM-97 are also included in the present invention. In certain embodiments, Antibodies having regions of at most 50%, at most 55%, at most 60%, at most 65%, at most 70%, at most 75%, at most 80%, at most 85%, at most 90%, at most 95% or at most 98% identity (as calculated using methods known in the art and described herein in Section 5.1.1) to the CDRs or variable regions of 2B8, FB1, 1F5, 2H7, 93-1B3, 109-3C2, B1, B9E9, 7D1, H147, L26, L27, and MEM-97 are also included in the present invention.

The present invention further encompasses the use of and compositions comprising an anti-CD20 antibody that has amino acid substitutions relative to a native anti-CD20 antibody that resulting in improved affinity for CD20 relative to the native antibody. In certain embodiments, such an antibody can be humanized. An exemplary method for identifying anti-CD20 antibodies with increased affinity is through systematic mutagenesis and screening, preferably reiterative screening, for antibodies with improved affinity to CD20, for example as described by Wu et al., 1998, Proc. Natl. Acad. Sci. U.S.A. 95:6037-6042.

Anti-CD20 antibodies useful in the methods and compositions of the present invention may also be described or specified in terms of their binding affinity to CD20. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×⁻¹³ M,10⁻¹³ M, 5×10⁻¹⁴ M, 10⁻¹⁴ M, 5×10⁻¹⁵ M, or 10⁻¹⁵ M. In certain embodiments, preferred binding affinities include those with a dissociation constant or Kd more than 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹, 5×10⁻¹² M, 10⁻¹² M, 5×⁻¹³ M, 10⁻¹³ M, 5×10⁻¹⁴ M, 10⁻¹⁴ M, 5×10⁻¹⁵ M, or 10⁻¹⁵ M.

The anti-CD20 antibodies useful in the present methods and compositions include derivatives that, in addition to conjugation to a drug of the invention, are modified, i.e, by the covalent attachment of any type of molecule to the antibody such that covalent attachment does not prevent the antibody from binding to CD20. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, synthesis in the presence of tunicamycin, etc. Additionally, the derivative may contain one or more non-classical amino acids.

The anti-CD20 antibodies useful in the methods and compositions of the present invention may be generated by any suitable method known in the art. Polyclonal antibodies to CD20 can be produced by various procedures well known in the art. For example, CD20 can be administered to various host animals including, but not limited to, rabbits, mice, rats, etc. to induce the production of sera containing polyclonal antibodies specific for the protein. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed., 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. In a non-limiting example, mice can be immunized with CD20 or a fragment or derivative thereof or with a cell expressing said CD20 or CD20 fragment or derivative. Once an immune response is detected, e.g., antibodies specific for CD20 are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding CD20. Ascites fluid, which generally contains high levels of antibodies, can be generated by injecting mice with positive hybridoma clones.

Antibody fragments which recognize specific epitopes may be generated by known techniques. For example, Fab and F(ab′)₂ fragments may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

For example, the anti-CD20 antibodies useful in the methods and compositions of the present invention can also be generated using various phage display methods known in the art. In phage display methods, functional antibody domains are displayed on the surface of phage particles which carry the nucleic acid sequences encoding them. In a particular embodiment, such phage can be utilized to display antigen binding domains expressed from a repertoire or combinatorial antibody library (e.g., human or murine). In phage display methods, functional antibody domains are displayed on the surface of phage particles which carry the nucleic acid sequences encoding them. In particular, DNA sequences encoding V_{H} and V_{L} domains are amplified from animal cDNA libraries (e.g., human or murine cDNA libraries of lymphoid tissues). The DNA encoding the V_{H} and V_{L} domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 binding domains expressed from phage with Fab, Fv or disulfide stabilized Fv antibody domains recombinantly fused to either the phage gene III or gene VIII protein. Phage expressing an antigen binding domain that binds to CD20 can be selected or identified with antigen e.g., using labeled antigen or antigen bound or captured to a solid surface or bead. Examples of phage display methods that can be used to make the anti-CD20 antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology, 191-280; PCT Application No. PCT/GB91/O1 134; PCT Publications WO 90/02809; WO 91/10737; WO 92/01047; WO 92/18619; WO 93/1 1236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques 1992, 12(6):864-869; and Sawai et al., 1995, AJRI 34:26-34; and Better et al., 1988, Science 240:1041-1043 (said references incorporated by reference in their entireties).

Examples of techniques which can be used to produce single-chain Fvs and antibodies include those described in U.S. Pat. Nos. 4,946,778 and 5,258,498; Huston et al., 1991, Methods in Enzymology 203:46-88; Shu et al., 1993, PNAS 90:7995-7999; and Skerra et al., 1988, Science 240:1038-1040. For some uses, including in vivo use of antibodies in humans and in vitro proliferation or cytotoxicity assays, it is preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science, 1985, 229:1202; Oi et al., 1986, BioTechniques 4:214; Gillies et al., 1989, J. Immunol. Methods 125:191-202; U.S. Pat. Nos. 5,807,715; 4,816,567; and 4,816,397, which are incorporated herein by reference in their entirety. Humanized antibodies are antibody molecules from non-human species antibody that binds the desired antigen having one or more CDRs from the non-human species and framework and constant regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., 1988, Nature 332:323, which are incorporated herein by reference in their entireties.) Antibodies can be humanized using a variety of techniques known in the art including, for example, CDR-grafting (EP 239,400; PCT publication WO 9 1/09967; U.S. Pat. Nos. 5,225,539; 5,530,101; and 5,585,089), veneering or resurfacing (EP 592,106; EP 519,596; Padlan, Molecular Immunology, 1991, 28(4/5):489-498; Studnicka et al., 1994, Protein Engineering 7(6):805-814; Roguska. et al., 1994, PNAS 91:969-973), and chain shuffling (U.S. Pat. No. 5,565,332).

Completely human antibodies are particularly desirable for therapeutic treatment of human subjects. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which express human immunoglobulin genes. For example, the human heavy and light chain immunoglobulin gene complexes may be introduced randomly or by homologous recombination into mouse embryonic stem cells. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of CD20. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see, Lonberg and Huszar, 1995, Int. Rev. Immunol. 13:65-93. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.), Genpharm (San Jose, Calif.) and Medarex (Princeton, N.J.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1994, Bio/technology 12:899-903).

In a specific embodiment, the anti-CD20 antibody is a bispecific antibody. In another specific embodiment, the anti-CD20 antibody is not a bispecific antibody.

In certain embodiments, the antibody is conjugated to a radioisotope. In more specific embodiments, the radioisotope is ⁹⁰Y (yttrium), ¹¹¹In (indium), ²¹¹At (astatide), ¹³¹I (iodine), ²¹²Bi (bismuth), ²¹³Bi, ²²⁵Ac (actinium), ¹⁸⁶Re (rhenium), ¹⁸⁸Re, ¹⁰⁹Pd (palladium), ⁶⁷Cu (copper), ⁷⁷Br (bromine), 105Rh (rhodium), ¹⁹⁸Au (gold), ¹⁹⁹Au or ²¹²Pb (lead).

The anti-CD20 antibodies useful in the present methods and compositions may further be recombinantly fused to a heterologous protein at the N- or C-terminus.

In specific embodiments of the invention the anti-CD20 antibody is not one or more of C2B8, 1F5, FB1, 2H7, 93-1B3, 109-3C2, B1, B9E9, 7D1, H147, L26, L27, or MEM97. In specific embodiments, the anti-CD20 antibody is a peptide that binds specifically to CD20.

### 5.1.1 Determining Sequence Homology Among Anti-CD20 Antibodies

To determine the percent identity of two amino acid sequences or of two nucleic acids, e.g., between the amino acid sequences of, or nucleic acid sequences that encode, the variable regions of two anti-CD20 antibodies, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment, the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid encoding an anti-CD20 antibody. BLAST protein searches can be performed with the XBLAST program, score=50, wordlength=3 to obtain amino acid sequences homologous to a an anti-CD20 antibody. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see http://bioweb.pasteur.fr/docs/man/man/fasta.1.html#sect2, the contents of which are incorporated herein by reference.

Alternatively, protein sequence alignment may be carried out using the CLUSTAL W algorithm, as described by Higgins et al., 1996, Methods Enzymol. 266:383-402.

The percent identity between two sequences can be determined using techniques similar to those described above, with or without allowing gaps. In calculating percent identity, only exact matches are counted.

### 5.2 Binding Assays

Methods of demonstrating the ability of an antibody to bind to CD20, and thus its usefulness in the disclosed methods and compositions, are described herein.

A putative anti-CD20 antibody may be assayed for immunospecific binding to CD20 by any method known in the art. The immunoassays which can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et. al., eds., 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody to the cell lysate, incubating for a period of time (e.g., 1-4 hours) at 40° C., adding protein A and/or protein G sepharose beads to the cell lysate, incubating for about an hour or more at 40° C., washing the beads in lysis buffer and resuspending the beads in SDS/sample buffer. The ability of the antibody to immunoprecipitate CD20 can be assessed by, e.g., Western blot analysis. One of skill in the art would be knowledgeable as to the parameters that can be modified to increase the binding of the antibody to CD20 and decrease the background (e.g., pre-clearing the cell lysate with sepharose beads). For further discussion regarding immunoprecipitation protocols see, e.g., Ausubel et al., eds., 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, incubating the membrane in blocking solution (e.g., PBS with 3% BSA or non-fat milk), washing the membrane in washing buffer (e.g., PBS-Tween 20), blocking the membrane with primary antibody (i.e., the putative anti-CD20 antibody) diluted in blocking buffer, washing the membrane in washing buffer, incubating the membrane with a secondary antibody (which recognizes the primary antibody, e.g., an anti-human antibody) conjugated to an enzyme substrate (e.g., horseradish peroxidase or alkaline phosphatase) or radioactive molecule (e.g., ³²P or ¹²⁵I) diluted in blocking buffer, washing the membrane in wash buffer, and detecting the presence of the secondary antibody. One of skill in the art would be knowledgeable as to the parameters that can be modified to increase the signal detected and to reduce the background noise. For further discussion regarding Western blot protocols see, e.g., Ausubel et al., eds., 1994, Current Protocols in Molecular Biology, Vol. 1, John Wiley & Sons, Inc., New York at 10.8.1.

ELISAs comprise preparing antigen (i.e., CD20), coating the well of a 96 well microtiter plate with the CD20, adding the antibody conjugated to a detectable compound such as an enzyme (e.g., horseradish peroxidase or alkaline phosphatase) to the well and incubating for a period of time, and detecting the presence of the antibody. In ELISAs the antibody does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of CD20 protein to the coated well. One of skill in the art would be knowledgeable as to the parameters that can be modified to increase the signal detected as well as other variations of ELISAs known in the art. For further discussion regarding ELISAs see, e.g., Ausubel et al., eds., 1994, Current Protocols in Molecular Biology, Vol. 1, John Wiley & Sons, Inc., New York at 11.2.1.

The binding affinity of an antibody to CD20 and the off-rate of an antibody-CD20 interaction can be determined by competitive binding assays. One example of a competitive binding assay is a radioimmunoassay comprising the incubation of labeled CD20 (e.g., ³H or ¹²⁵I) with the antibody of interest in the presence of increasing amounts of unlabeled CD20, and the detection of the antibody bound to the labeled CD20. The affinity of the antibody for CD20 and the binding off-rates can then be determined from the data by Scatchard plot analysis. Competition with a second antibody can also be determined using radioimmunoassays. In this case, CD20 is incubated with the antibody of interest conjugated to a labeled compound (e.g., ³H or ¹²⁵I) in the presence of increasing amounts of an unlabeled second antibody.

### 5.3 Methods of Producing Anti-CD20 Antibodies

The anti-CD20 antibodies of the invention can be produced by any method known in the art for the synthesis of proteins, in particular, by chemical synthesis or preferably, by recombinant expression techniques.

Recombinant expression of an anti-CD20 antibody, including a fragment, derivative or analog thereof, e.g., a heavy or light chain of an anti-CD20 antibody, requires construction of an expression vector containing a nucleic acid that encodes the anti-CD20 antibody. Once a nucleic acid encoding an anti-CD20 antibody has been obtained, the vector for the production of the anti-CD20 antibody may be produced by recombinant DNA technology using techniques well known in the art. Thus, methods for preparing an anti-CD20 antibody by expressing a nucleic acid containing a nucleotide sequence encoding said anti-CD20 antibody are described herein. Methods which are well known to those skilled in the art can be used to construct expression vectors containing coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an anti-CD20 antibody operably linked to a promoter. The anti-CD20 antibody nucleotide sequence may encode a heavy or light chain thereof, or a heavy or light chain variable domain, operably linked to a promoter. Such vectors may include the nucleotide sequence encoding the constant region of the anti-CD20 antibody molecule (see, e.g., PCT Publication WO 86/05807; PCT Publication WO 89/01036; and U.S. Pat. No. 5,122,464) and the variable domain of the anti-CD20 antibody may be cloned into such a vector for expression of the entire heavy or light chain.

The expression vector is transferred to a host cell by conventional techniques and the transfected cells are then cultured by conventional techniques to produce a protein of the invention. Thus, the invention encompasses host cells containing a nucleic acid encoding a protein of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the protein molecules of the invention. Such host-expression systems represent vehicles by which the coding sequences of interest may be produced and subsequently purified, but also represent cells which may, when transformed or transfected with the appropriate nucleotide coding sequences, express a protein of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecules, are used for the expression of a recombinant protein of the invention. For example, mammalian cells such as Chinese hamster ovary cells (CHO), in conjunction with a vector such as the major intermediate early gene promoter element from human cytomegalovirus is an effective expression system for proteins of the invention (Foecking et al., 1986, Gene 45:101; Cockett et al., 1990, Bio/Technology 8:2).

In bacterial systems, a number of expression vectors may be advantageously selected depending upon the use intended for the folding and post-translation modification requirements protein being expressed. Where possible, when a large quantity of an anti-CD20 antibody is to be produced, for the generation of the anti-CD20 ADCs of the invention or pharmaceutical compositions comprising such ADCs, vectors which direct the expression of high levels of fusion protein products that are readily purified may be desirable. Such vectors include, but are not limited, to the E. coli expression vector pUR278 (Ruther et al., 1983, EMBO 1. 2:1791), in which the anti-CD20 antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express fusion proteins with glutathione S-transferase (GST). In general, such fusion proteins are soluble and can easily be purified from lysed cells by adsorption and binding to matrix glutathioneagarose beads followed by elution in the presence of free glutathione. The pGEX vectors are designed to include thrombin or factor Xa protease cleavage sites so that the cloned anti-CD20 antibody can be released from the GST moiety.

In an insect system, Autographa californica nuclear polyhedrosis virus (AcNPV) is used as a vector to express foreign genes. The virus grows in Spodoptera frugiperda cells. The anti-CD20 antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the coding sequence of the anti-CD20 antibody may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the anti-CD20 antibody in infected hosts. (See, e.g., Logan & Shenk, 1984, Proc. Natl. Acad. Sci. USA 8 1:355-359). Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of anti-CD20 antibodies may be important for the binding and/or activities of the antibodies. Different host cells have characteristic and specific mechanisms for the post-translational processing and modification of proteins and gene products. Appropriate cell lines or host systems can be chosen to ensure the correct modification and processing of the anti-CD20 antibody expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, Hela, COS, MDCK, 293, 3T3, and W138.

For long-term, high-yield production of recombinant anti-CD20 antibodies, stable expression is preferred. For example, cell lines which stably express an anti-CD20 antibody may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express an anti-CD20 antibody for use in the methods of the present invention.

A number of selection systems may be used, including but not limited to the herpes simplex virus thymidine kinase (Wigler et al., 1977, Cell 11:223), hypoxanthine guanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95 ; Tolstoshev, 1993, Ann. Rev. Pharmacol. Toxicol. 32:573-596; Mulligan, 1993, Science 260:926-932 ; and Morgan and Anderson, 1993, Ann. Rev. Biochem. 62:191-217; May, 1993, TIB TECH 11(5):155-215); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, New York (1993); Kriegler, Gene Transfer and Expression, A Laboratory Manual, Stockton Press, New York (1990); and in Chapters 12 and 13, Dracopoli et al. (eds), Current Protocols in Human Genetics, John Wiley & Sons, New York (1994); Colberre-Garapin et al., 1981, J. Mol. Biol. 150:1, which are incorporated by reference herein in their entireties.

The expression levels of an anti-CD20 antibody can be increased by vector amplification (for a review, see Bebbington and Hentschel, “The Use of Vectors Based on Gene Amplification for the Expression of Cloned Genes in Mammalian Cells in DNY Cloning”, Vol. 3. (Academic Press, New York, 1987)). When a marker in the vector system expressing the anti-CD20 antibody is amplifiable, increase in the level of inhibitor present in culture of host cell will increase the number of copies of the marker gene. Since the amplified region is associated with the anti-CD20 antibody gene, production of the anti-CD20 antibody will also increase (Crouse et al., 1983, Mol. Cell. Biol. 3:257).

In certain specific embodiments, the host cell may be co-transfected with two expression vectors encoding an anti-CD20 antibody, the first vector encoding a heavy chain derived protein and the second vector encoding a light chain derived protein. The two vectors may contain identical selectable markers which enable equal expression of heavy and light chain proteins. Alternatively, a single vector may be used which encodes, and is capable of expressing, both heavy and light chain proteins. In such situations, the light chain should be placed before the heavy chain to avoid an excess of toxic free heavy chain (Proudfoot, 1986, Nature 322:52 (1986); Kohler, 1980, Proc. Natl. Acad. Sci. USA 77:2 197). The coding sequences for the heavy and light chains may comprise cDNA or genomic DNA.

Once an anti-CD20 antibody has been produced by an animal, chemically synthesized, or recombinantly expressed, it may be purified by any method known in the art for purification of proteins, for example, by chromatography (e.g., ion exchange; affinity, particularly by affinity for the specific antigen (i.e., CD20); Protein A; or affinity for a heterologous fusion partner wherein the protein is a fusion protein; and sizing column chromatography), centrifugation, differential solubility, or by any other standard technique for the purification of proteins.

The present invention encompasses the use of anti-CD20 antibodies recombinantly fused or chemically conjugated (including both covalent and non-covalent conjugation) to heterologous proteins (of preferably at least 10, 20, 30, 40, 50, 60, 70, 80, 90 or at least 100 amino acids) to generate fusion proteins. The fusion does not necessarily need to be direct, but may occur through linker sequences.

### 5.4 Linkers

As discussed below in Section 5.6, ADCs are generally made by conjugating a drug to an antibody through a linker. Thus, a majority of the ADCs of the present invention, which comprise an anti-CD20 antibody and a high potency drug and/or an internalization-promoting drug, further comprise a linker. Any linker that is known in the art may be used in the ADCs of the present invention, e.g., bifunctional agents (such as dialdehydes or imidoesters) or branched hydrazone linkers (see, e.g., U.S. Pat. No. 5,824,805, which is incorporated by reference herein in its entirety).

In certain, non-limiting, embodiments of the invention, the linker region between the drug moiety and the antibody moiety of the anti-CD20 ADC is cleavable or hydrolyzable under certain conditions, wherein cleavage or hydrolysis of the linker releases the drug moiety from the antibody moiety. Preferably, the linker is sensitive to cleavage or hydrolysis under intracellular conditions.

In a preferred embodiment, the linker region between the drug moiety and the antibody moiety of the anti-CD20 ADC is hydrolyzable if the pH changes by a certain value or exceeds a certain value. In a particularly preferred embodiment of the invention, the linker is hydrolyzable in the milieu of the lysosome, e.g., under acidic conditions (i.e., a pH of around 5-5.5 or less). In other embodiments, the linker is a peptidyl linker that is cleaved by a peptidase or protease enzyme, including but not limited to a lysosomal protease enzyme, a membrane-associated protease, an intracellular protease, or an endosomal protease. Preferably, the linker is at least two amino acids long, more preferably at least three amino acids long. Peptidyl linkers that are cleavable by enzymes that are present in CD20-expressing cancers are preferred. For example, a peptidyl linker that is cleavable by cathepsin-B (e.g., a Gly-Phe-Leu-Gly linker), a thiol-dependent protease that is highly expressed in cancerous tissue, can be used. Other such linkers are described, e.g., in U.S. Pat. No. 6,214,345, which is incorporated by reference in its entirety herein.

In other, non-mutually exclusive embodiments of the invention, the linker by which the anti-CD20 antibody and the drug of an ADC of the invention are conjugated promotes cellular internalization. In certain embodiments, the linker-drug moiety of the ADC promotes cellular internalization. In certain embodiments, the linker is chosen such that the structure of the entire ADC promotes cellular internalization.

In a specific embodiment of the invention, derivatives of valine-citrulline are used as linker (val-cit linker). The synthesis of doxorubicin with the val-cit linker have been previously described (U.S. Pat. No. 6,214,345 to Dubowchik and Firestone, which is incorporated by reference herein in its entirety).

In another specific embodiment, the linker is a phe-lys linker.

In another specific embodiment, the linker is a thioether linker (see, e.g., U.S. Pat. No. 5,622,929 to Willner et al., which is incorporated by reference herein in its entirety).

In yet another specific embodiment, the linker is a hydrazone linker (see, e.g., U.S. Pat. Nos. 5,122,368 to Greenfield et al. and 5,824,805 to King et al., which are incorporated by reference herein in their entireties).

In yet other specific embodiments, the linker is a disulfide linker. A variety of disulfide linkers are known in the art, including but not limited to those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene). SPDB and SMPT (see, e.g., Thorpe et al., 1987, Cancer Res., 47:5924-5931; Wawrzynczak et al., 1987, In Immunoconjugates: Antibody Conjugates in Radioimagery and Therapy of Cancer, ed. C. W. Vogel, Oxford U. Press, pp. 28-55; see also U.S. Pat. No. 4,880,935 to Thorpe et al., which is incorporated by reference herein in its entirety).

A variety of linkers that can be used with the compositions and methods of the present invention are described in U.S. Patent application entitled “Drug Conjugates and their use for treating cancer, an autoimmune disease or an infectious disease”, by Inventors: Peter D. Senter, Svetlana Doronina and Brian E. Toki, submitted on event day herewith, which is incorporated by reference in its entirety herein.

In yet other embodiments of the present invention, the linker unit of an anti-CD20 antibody-linker-drug conjugate (anti-CD20 ADC) links the cytotoxic or cytostatic agent (drug unit; -D) and the anti-CD20 antibody unit (-A). As used herein the term anti-CD20 ADC encompasses anti-CD20 antibody drug conjugates with and without a linker unit. The linker unit has the general formula:
-Tₐ—W_{w}—Y_{y}—
wherein:

-T- is a stretcher unit;

a is 0 or 1;

each —W— is independently an amino acid unit;

w is independently an integer ranging from 2 to 12;

—Y— is a spacer unit; and

y is 0, 1 or 2.

### 5.4.1 The Stretcher Unit

The stretcher unit (-T-), when present, links the anti-CD20 antibody unit to an amino acid unit (—W—). Useful functional groups that can be present on an anti-CD20 antibody, either naturally or via chemical manipulation include, but are not limited to, sulfhydryl, amino, hydroxyl, the anomeric hydroxyl group of a carbohydrate, and carboxyl. Preferred functional groups are sulfhydryl and amino. Sulfhydryl groups can be generated by reduction of the intramolecular disulfide bonds of an anti-CD20 antibody. Alternatively, sulfhydryl groups can be generated by reaction of an amino group of a lysine moiety of an anti-CD20 antibody with 2-iminothiolane (Traut's reagent) or other sulfhydryl generating reagents. In specific embodiments, the anti-CD20 antibody is a recombinant antibody and is engineered to carry one or more lysines. In other embodiments, the recombinant anti-CD20 antibody is engineered to carry additional sulfhydryl groups, e.g., additional cysteines.

In certain specific embodiments, the stretcher unit forms a bond with a sulfur atom of the anti-CD20 antibody unit. The sulfur atom can be derived from a sulfhydryl (—SH) group of a reduced anti-CD20 antibody (A). Representative stretcher units of these embodiments are depicted within the square brackets of Formulas (Ia) and (Ib; see infra), wherein A-, —W—, —Y—, -D, w and y are as defined above and R¹ is selected from —C₁-C₁₀ alkylene-, —C₃-C₈ carbocyclo-, —O—(C₁-C₈alkyl)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —(CH₂CH₂O)ᵣ—, and —(CH₂CH₂O)ᵣ—CH₂—; and r is an integer ranging from 1-10.

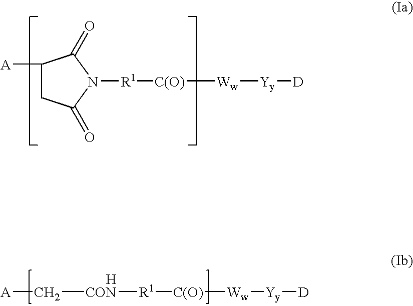

An illustrative stretcher unit is that of formula (Ia) where R¹ is —(CH₂)₅—:

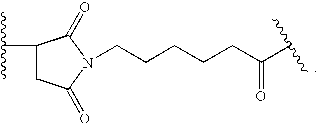

Another illustrative stretcher unit is that of formula (Ia) where R¹ is —(CH₂CH₂O)ᵣ—CH₂—; and r is 2:

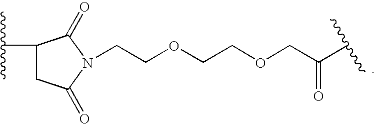

Still another illustrative stretcher unit is that of formula (Ib) where R¹ is —(CH₂)₅—:

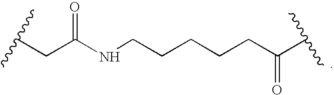

In certain other specific embodiments, the stretcher unit is linked to the anti-CD20 antibody unit (A) via a disulfide bond between a sulfur atom of the anti-CD20 antibody unit and a sulfur atom of the stretcher unit. A representative stretcher unit of this embodiment is depicted within the square brackets of Formula (II), wherein R¹, A-, —W—, —Y—, -D, w and y are as defined above.
A [symbol] S—R¹—C(O) [symbol] W_{w}—Y_{y}—D (II)

In even other specific embodiments, the reactive group of the stretcher contains a reactive site that can be reactive to an amino group of an anti-CD20 antibody. The amino group can be that of an arginine or a lysine. Suitable amine reactive sites include, but are not limited to, activated esters such as succinimide esters, 4-nitrophenyl esters, pentafluorophenyl esters, anhydrides, acid chlorides, sulfonyl chlorides, isocyanates and isothiocyanates. Representative stretcher units of these embodiments are depicted within the square brackets of Formulas (IIIa) and (IIIb), wherein R¹, A-, —W—, —Y—, -D, w and y are as defined above;

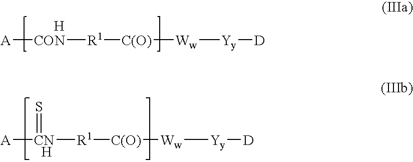

In yet another aspect of the invention, the reactive function of the stretcher contains a reactive site that is reactive to a modified carbohydrate group that can be present on an anti-CD20 antibody. In a specific embodiment, the anti-CD20 antibody is glycosylated enzymatically to provide a carbohydrate moiety. The carbohydrate may be mildly oxidized with a reagent such as sodium periodate and the resulting carbonyl unit of the oxidized carbohydrate can be condensed with a stretcher that contains a functionality such as a hydrazide, an oxime, a reactive amine, a hydrazine, a thiosemicarbazone, a hydrazine carboxylate, and an arylhydrazide such as those described by Kaneko, T. et al. Bioconjugate Chem 1991, 2, 133-41. Representative stretcher units of this embodiment are depicted within the square brackets of Formulas (IVa)-(IVc), wherein R¹, A-, —W—, —Y—, -D, w and y are as defined above.

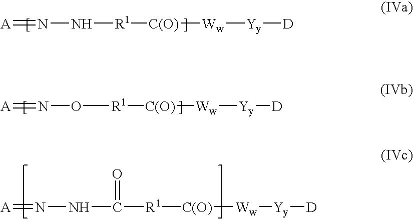

### 5.4.2 The Amino Acid Unit

The amino acid unit (—W—) links the stretcher unit (-T-) to the Spacer unit (—Y—) if the Spacer unit is present, and links the stretcher unit to the cytotoxic or cytostatic agent (Drug unit; D) if the spacer unit is absent.

—W_{w}— is a dipeptide, tripeptide, tetrapeptide, pentapeptide, hexapeptide, heptapeptide, octapeptide, nonapeptide, decapeptide, undecapeptide or dodecapeptide unit. Each —W— unit independently has the formula denoted below in the square brackets, and w is an integer ranging from 2 to 12: 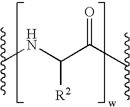
wherein R² is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

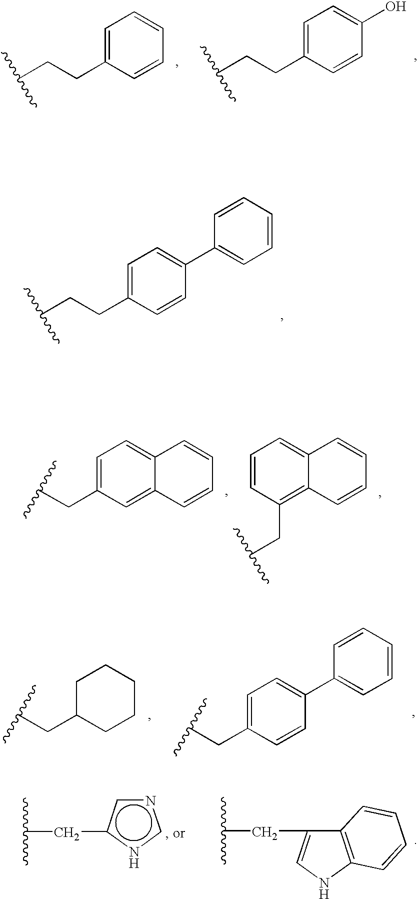

The amino acid unit of the linker unit can be enzymatically cleaved by an enzyme including, but not limited to, a tumor-associated protease to liberate the drug unit (—D) which is protonated in vivo upon release to provide a cytotoxic drug (D).

Illustrative W_{w} units are represented by formulas (V)-(VII):

| | |
|---|---|
| | (V) |
| 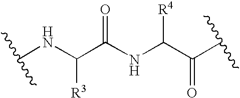 | |

wherein R³ and R⁴ are as follows:

| R³ | R⁴ |
|---|---|
| benzyl | (CH₂)₄NH₂; |
| methyl | (CH₂)₄NH₂; |
| isopropyl | (CH₂)₄NH₂; |
| isopropyl | (CH₂)₃NHCONH₂; |
| benzyl | (CH₂)₃NHCONH₂; |
| isobutyl | (CH₂)₃NHCONH₂; |
| sec-butyl | (CH₂)₃NHCONH_{2L ;} |
| 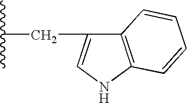 | (CH₂)₃NHCONH₂; |
| benzyl | methyl; and |
| benzyl | (CH₂)₃NHC(═NH)NH₂; |

| | |
|---|---|
| | (VI) |
| 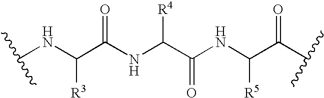 | |

wherein R³, R⁴ and R⁵ are as follows:

| R³ | R⁴ | R⁵ |
|---|---|---|
| benzyl | benzyl | (CH₂)₄NH₂; |
| isopropyl | benzyl | (CH₂)₄NH₂; and |
| H | benzyl | (CH₂)₄NH₂; |

| | |
|---|---|
| | (VII) |
| 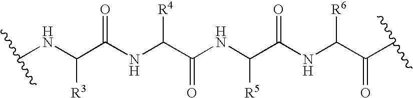 | |

wherein R³, R⁴, and R⁵ and R⁶ are as follows:

| R³ | R⁴ | R⁵ | R⁶ |
|---|---|---|---|
| H | benzyl | isobutyl | H; and |
| methyl | isobutyl | methyl | isobutyl. |

Preferred amino acid units include, but are not limited to, units of formula (V) where: R³ is benzyl and R⁴ is —(CH₂)₄NH₂; R³is isopropyl and R⁴ is —(CH₂)₄NH₂; R³ is isopropyl and R⁴ is —(CH₂)₃NHCONH₂. Another preferred amino acid unit is a unit of formula (VI), where: R³ is benzyl, R⁴ is benzyl, and R⁵ is —(CH₂)₄NH₂.

—W_{w}— units useful in the present invention can be designed and optimized in their selectivity for enzymatic cleavage by a particular tumor-associated protease. The preferred —W_{w}— units are those whose cleavage is catalyzed by the proteases, cathepsin B, C and D, and plasmin.

In one embodiment, —W_{w}— is a dipeptide, tripeptide or tetrapeptide unit.

Where R², R³, R⁴, R⁵ or R⁶ is other than hydrogen, the carbon atom to which R², R³, R⁴, R⁵ or R⁶ is attached is chiral.

Each carbon atom to which R², R³, R⁴, R⁵ or R⁶ is attached is independently in the (S) or (R) configuration.

In a preferred embodiment, the amino acid unit is a phenylalanine-lysine dipeptide (phe-lys linker). In antother preferred embodiment, the amino acid unit is a valine-citrulline dipeptide (val-cit linker).

### 5.4.3 The Spacer Unit

The spacer unit (—Y—), when present, links an amino acid unit to the drug unit. Spacer units are of two general types: self-immolative and non self-immolative. A non self-immolative spacer unit is one in which part or all of the spacer unit remains bound to the drug unit after enzymatic cleavage of an amino acid unit from the anti-CD20 antibody-linker-drug conjugate or the drug-linker compound. Examples of a non self-immolative spacer unit include, but are not limited to a (glycine-glycine) spacer unit and a glycine spacer unit (both depicted in Scheme 1). When an anti-CD20 antibody-linker-drug conjugate of the invention containing a glycine-glycine spacer unit or a glycine spacer unit undergoes enzymatic cleavage via a tumor-cell associated-protease, a cancer-cell-associated protease or a lymphocyte-associated protease, a glycine-glycine-drug moiety or a glycine-drug moiety is cleaved from A-T—W_{w}—. To liberate the drug, an independent hydrolysis reaction should take place within the target cell to cleave the glycine-drug unit bond.

In a preferred embodiment, —Y_{y}— is a p-aminobenzyl ether which can be substituted with Qₘ where Q is is —C₁-C₈ alkyl, —C₁-C₈ alkoxy, -halogen, -nitro or -cyano; and m is an integer ranging from 0-4.

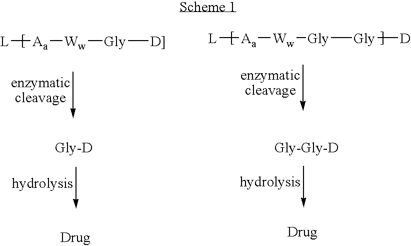

In one embodiment, a non self-immolative spacer unit (—Y—) is -Gly-Gly-.

In another embodiment, a non self-immolative the spacer unit (—Y—) is -Gly-.

In one embodiment, the drug-linker compound or an anti-CD20 antibody-linker-drug conjugate lacks a spacer unit (y=0).

Alternatively, an anti-CD20 antibody-linker-drug conjugate of the invention containing a self-immolative spacer unit can release the drug (D) without the need for a separate hydrolysis step. In these embodiments, —Y— is a p-aminobenzyl alcohol (PAB) unit that is linked to —W_{w}— via the nitrogen atom of the PAB group, and connected directly to -D via a carbonate, carbamate or ether group (Scheme 2 and Scheme 3). 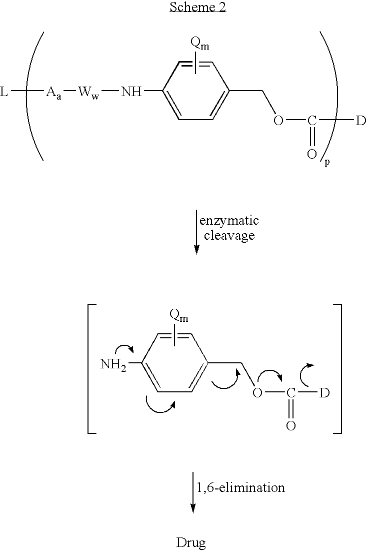
where Q is —C₁-C₈ alkyl, —C₁-C₈ alkoxy, -halogen, -nitro or -cyano; m is an integer ranging from 0-4; and p is an integer ranging from 1-20. 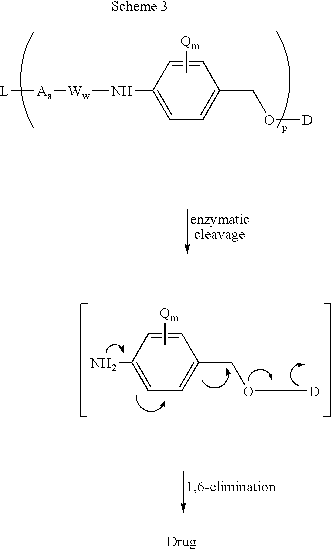
where Q is —C₁-C₈ alkyl, —C₁-C₈ alkoxy, -halogen, -nitro or -cyano; m is an integer ranging from 0-4; and p is an integer ranging from 1-20.

Other examples of self-immolative spacers include, but are not limited to, aromatic compounds that are electronically equivalent to the PAB group such a 2-aminoimidazol-5-methanol derivatives (see Hay et al., Bioorg. Med. Chem. Lett., 1999, 9, 2237 for examples) and ortho or para-aminobenzylacetals. Spacers can be used that undergo facile cyclization upon amide bond hydrolysis, such as substituted and unsubstituted 4-aminobutyric acid amides (Rodrigues et al., Chemistry Biology, 1995, 2, 223), appropriately substituted bicyclo[2.2.1] and bicyclo[2.2.2] ring systems (Storm, et al., J. Amer. Chem. Soc., 1972, 94, 5815) and 2-aminophenylpropionic acid amides (Amsberry, et al., J. Org. Chem., 1990, 55, 5867). Elimination of amine-containing drugs that are substituted at the a-position of glycine (Kingsbury, et al., J. Med. Chem., 1984, 27, 1447) are also examples of self-immolative spacer strategies that can be applied to the anti-CD20 antibody-linker-drug conjugates of the invention.

In an alternate embodiment, the spacer unit is a branched bis(hydroxymethyl)styrene (BHMS) unit (Scheme 4), which can be used to incorporate additional drugs. 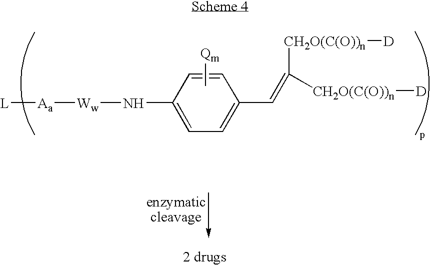
where Q is —C₁-C₈ alkyl, —C₁-C₈ alkoxy, -halogen, -nitro or -cyano; m is an integer ranging from 0-4; n is 0 or 1; and p is an integer raging from 1-20.

In one embodiment, the two -D moieties are the same.

In another embodiment, the two -D moieties are different.

Preferred spacer units (—Y_{y}—) are represented by Formulas (VIII)-(X): 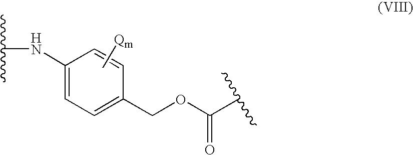
where Q is C₁-C₈ alkyl, C₁-C₈ alkoxy, halogen, nitro or cyano; and m is an integer ranging from 0-4;

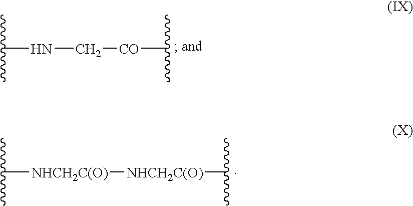

### 5.5 Drugs

The present invention encompasses compositions comprising ADCs comprising an anti-CD20 antibody conjugated to a drug through a linker, where the ADC is capable of exerting a cytotoxic or cytostatic effect on a CD20-expressing cell by the drug. As used herein, the term “drug” or “cytotoxic agent,” where employed in the context of an anti-CD20 ADC of the invention, does not include radioisotopes.

The ADCs of the invention are tailored to produce clinically beneficial cytotoxic or cytostatic effects on CD20-expressing cells when administered to a patient with a CD20-expressing cancer or an immune disorder involving CD20-expressing cells, preferably when administered alone but also in combination with other therapeutic agents. Such cytotoxic or cytostatic effects can be achieved by use of a high potency drug or a drug that is capable of enhancing the rate of accumulation inside the CD20-expressing cell of the anti-CD20 antibody to which it is conjugated. Examples of such classes of drugs, which are not mutually exclusive, are provided below.

### 5.5.1 High Potency Drugs

The present invention encompasses the use of anti-CD20 ADCs in which the cytotoxic or cytostatic agent is a high potency drug, i.e., a drug that has a sufficiently high degree of potency that the ADC is capable of exerting a cytotoxic or cytotoxic effect on CD20-expressing cells, such as CD20-expressing cancer cells or CD20-expressing cells involved in an immune disorder.

In certain, preferred, embodiments of the present invention, a high potency drug is one that is at least 60-fold more potent than doxorubicin. In certain other embodiments the high potency drug is at least 10-fold, 20-fold, 30-fold, 40-fold, 50-fold, 70-fold, 80-fold, 90-fold, 100-fold, 125-fold, 150-fold, 175-fold, 200-fold, 250-fold, 500-fold, 750-fold, 1,000-fold, 2,000-fold, or 20,000-fold more potent than doxorubicin. In certain embodiments of the invention, a high potency drug is one that is not more than 20-fold, 30-fold, 40-fold, 50-fold, 60-fold, 70-fold, 80-fold, 90-fold, 100-fold, 125-fold, 150-fold, 175-fold, 200-fold, 250-fold, 500-fold, 750-fold, 1,000-fold, 2,000-fold, or 20,000-fold more potent than doxorubicin. In certain embodiments of the invention, a high potency drug is one that is between 60-fold and 2000-fold, 60-fold and 1000-fold, 60-fold and 500-fold, 100-fold and 250-fold, 40-fold and 4,000-fold, 10-fold and 20-fold, 20-fold and 30-fold, 30-fold and 40-fold, 40-fold and 50-fold, 50-fold and 60-fold, 60-fold and 70-fold, 70-fold and 80-fold, 80-fold and 90-fold, 90-fold and 100-fold, 100-fold and 125-fold, 125-fold and 150-fold, 150-fold and 175-fold, 175-fold and 200-fold, 200-fold and 250-fold, 250-fold and 500-fold, 500-fold and 750-fold, 750-fold and 1,000-fold, 1,000-fold and 2,000-fold, 10-fold and 20,000-fold, 20-fold and 10,000-fold, 50-fold and 5,000-fold, 75-fold and 2,000-fold, 100-fold and 1,000-fold, 200-fold and 500-fold, 10-fold and 100-fold, 100-fold and 200-fold, 200-fold and 500-fold, or 500-fold and 2,000-fold more potent than doxorubicin. Methods by which cytotoxicity, an indicator of potency, is measured are described in Section 5.4.1.1, infra.

In certain embodiments of the invention, potency is determined by comparing the effect of the ADC comprising the high potency drug to an ADC comprising the same anti-CD20 antibody and doxorubicin on the same cell type. In these embodiments, a high potency drug is one that, when conjugated to an anti-CD20 antibody by a certain linker, is at least 10-fold, 20-fold, 30-fold, 40-fold, 50-fold, 60-fold, 70-fold, 80-fold, 90-fold, 100-fold, 125-fold, 150-fold, 175-fold, 200-fold, 250-fold, 500-fold, 750-fold, 1000-fold, 2,000-fold, or 20,000-fold more cytotoxic or cytostatic than an ADC comprising the same anti-CD20 antibody conjugated to doxorubicin. In these embodiments, a high potency drug is one that, when conjugated to an anti-CD20 antibody by a certain linker, is not more than 20-fold, 30-fold, 40-fold, 50-fold, 60-fold, 70-fold, 80-fold, 90-fold, 100-fold, 125-fold, 150-fold, 175-fold, 200-fold, 250-fold, 500-fold, 750-fold, 1000-fold, 2,000-fold, or 20,000-fold more cytotoxic or cytostatic than an ADC comprising the same anti-CD20 antibody conjugated to doxorubicin. In these embodiments, a high potency drug is one that when conjugated to an anti-CD20 antibody by a certain linker, is between 60-fold and 2000-fold, 60-fold and 1000-fold, 60-fold and 500-fold, 100-fold and 250-fold, 40-fold and 4,000-fold, 10-fold and 20-fold, 20-fold and 30-fold, 30-fold and 40-fold, 40-fold and 50-fold, 50-fold and 60-fold, 60-fold and 70-fold, 70-fold and 80-fold, 80-fold and 90-fold, 90-fold and 100-fold, 100-fold and 125-fold, 125-fold and 150-fold, 150-fold and 175-fold, 175-fold and 200-fold, 200-fold and 250-fold, 250-fold and 500-fold, 500-fold and 750-fold, 750-fold and 1,000-fold, 1,000-fold and 2,000-fold, 10-fold and 20,000-fold, 20-fold and 10,000-fold, 50-fold and 5,000-fold, 75-fold and 2,000-fold, 100-fold and 1,000-fold, 200-fold and 500-fold, 10-fold and 100-fold, 100-fold and 200-fold, 200-fold and 500-fold, or 500-fold and 2,000-fold more cytotoxic or cytostatic than an ADC comprising the same anti-CD20 antibody conjugated to doxorubicin. Such comparisons should be made at comparable concentrations on the same cell types.

In other embodiments of the invention, potency is determined by comparing the effect of the drug to doxorubicin on the same cell type. In these embodiments, a high potency drug is one that is at least 10-fold, 20-fold, 30-fold, 40-fold, 50-fold, 60-fold, 70-fold, 80-fold, 90-fold, 100-fold, 125-fold, 150-fold, 175-fold, 200-fold, 250-fold, 500-fold, 750-fold, 1000-fold, 2,000-fold, or 20,000-fold more cytotoxic or cytostatic than doxorubicin. In these embodiments, a high potency drug is one that is not more than 20-fold, 30-fold, 40-fold, 50-fold, 60-fold, 70-fold, 80-fold, 90-fold, 100-fold, 125-fold, 150-fold, 175-fold, 200-fold, 250-fold, 500-fold, 750-fold, 1000-fold, 2,000-fold, or 20,000-fold more cytotoxic or cytostatic than doxorubicin. In these embodiments a high potency drug is one that is between 60-fold and 2000-fold, 60-fold and 1000-fold, 60-fold and 500-fold, 100-fold and 250-fold, 40-fold and 4,000-fold, 10-fold and 20-fold, 20-fold and 30-fold, 30-fold and 40-fold, 40-fold and 50-fold, 50-fold and 60-fold, 60-fold and 70-fold, 70-fold and 80-fold, 80-fold and 90-fold, 90-fold and 100-fold, 100-fold and 125-fold, 125-fold and 150-fold, 150-fold and 175-fold, 175-fold and 200-fold, 200-fold and 250-fold, 250-fold and 500-fold, 500-fold and 750-fold, 750-fold and 1,000-fold, 1,000-fold and 2,000-fold, 10-fold and 20,000-fold, 20-fold and 10,000-fold, 50-fold and 5,000-fold, 75-fold and 2,000-fold, 100-fold and 1,000-fold, 200-fold and 500-fold, 10-fold and 100-fold, 100-fold and 200-fold, 200-fold and 500-fold, or 500-fold and 2,000-fold more cytotoxic or cytostatic than doxorubicin. Such comparisons should be made at comparable concentrations on the same cell types.

The IC₅₀ of a cytotoxic agent or an anti-CD20 antibody-cytotoxic agent conjugate, respectively, is the concentration of the cytotoxic agent or the anti-CD20 antibody-cytotoxic agent conjugate, respectively, at which 50% of cells in a cell culture or the anti-CD20 antibody-cytotoxic agent conjugate, respectively, are non-viable at the end of an incubation period of the cell culture with the cytotoxic agent or the anti-CD20 antibody-cytotoxic agent conjugate, respectively, compared to an untreated cell culture under otherwise the same conditions (see below) in the absence of a cytotoxic or cytostatic agent. Thus, the higher the cytotoxicity of an agent or an anti-CD20 antibody-cytotoxic agent conjugate, respectively, is, the lower is its IC₅₀.

In other embodiments of the invention, potency is determined by comparing the IC₅₀ of the cytotoxic agent with the IC₅₀ of doxorubicin on the same cell type. In these embodiments, a high potency cytotoxic agent is one that has an IC₅₀ of at least 10-fold, 20-fold, 30-fold, 40-fold, 50-fold, 60-fold, 70-fold, 80-fold, 90-fold, 100-fold, 125-fold, 150-fold, 175-fold, 200-fold, 250-fold, 500-fold, 750-fold, 1000-fold, 2,000-fold, or 20,000-fold less than the IC₅₀ of doxorubicin. In these embodiments, a high potency cytotoxic agent is one that has an IC₅₀ of not more than 20-fold, 30-fold, 40-fold, 50-fold, 60-fold, 70-fold, 80-fold, 90-fold, 100-fold, 125-fold, 150-fold, 175-fold, 200-fold, 250-fold, 500-fold, 750-fold, 1000-fold, 2,000-fold, or 20,000-fold less than the IC₅₀ of doxorubicin. In these embodiments, a high potency cytotoxic agent is one that has an IC₅₀ of between 60-fold and 2000-fold, 60-fold and 1000-fold, 60-fold and 500-fold, 100-fold and 250-fold, 40-fold and 4,000-fold, 10-fold and 20-fold, 20-fold and 30-fold, 30-fold and 40-fold, 40-fold and 50-fold, 50-fold and 60-fold, 60-fold and 70-fold, 70-fold and 80-fold, 80-fold and 90-fold, 90-fold and 100-fold, 100-fold and 125-fold, 125-fold and 150-fold, 150-fold and 175-fold, 175-fold and 200-fold, 200-fold and 250-fold, 250-fold and 500-fold, 500-fold and 750-fold, 750-fold and 1,000-fold, 1,000-fold and 2,000-fold, 10-fold and 20,000-fold, 20-fold and 10,000-fold, 50-fold and 5,000-fold, 75-fold and 2,000-fold, 100-fold and 1,000-fold, 200-fold and 500-fold, 10-fold and 100-fold, 100-fold and 200-fold, 200-fold and 500-fold, or 500-fold and 2,000-fold less than the IC₅₀ of doxorubicin. Such comparisons should be made at comparable concentrations on the same cell types.

In yet other embodiments of the invention, potency is determined by comparing the IC₅₀ of an anti-CD20 ADC containing the cytotoxic agent with the IC₅₀ of an anti-CD20 ADC containing doxorubicin. In these embodiments, a high cytotoxic agent is one that has an IC₅₀ of at least 10-fold, 20-fold, 30-fold, 40-fold, 50-fold, 60-fold, 70-fold, 80-fold, 90-fold, 100-fold, 125-fold, 150-fold, 175-fold, 200-fold, 250-fold, 500-fold, 750-fold, 1000-fold, 2,000-fold, or 20,000-fold less than the IC₅₀ of an anti-CD20 ADC containing doxorubicin. In these embodiments, a high cytotoxic agent is one that has an IC₅₀ of not more than 20-fold, 30-fold, 40-fold, 50-fold, 60-fold, 70-fold, 80-fold, 90-fold, 100-fold, 125-fold, 150-fold, 175-fold, 200-fold, 250-fold, 500-fold, 750-fold, 1000-fold, 2,000-fold, or 20,000-fold less than the IC₅₀ of an anti-CD20 ADC containing doxorubicin. In these embodiments, a high cytotoxic agent is one that has an IC₅₀ of between 60-fold and 2000-fold, 60-fold and 1000-fold, 60-fold and 500-fold, 100-fold and 250-fold, 40-fold and 4,000-fold, 10-fold and 20-fold, 20-fold and 30-fold, 30-fold and 40-fold, 40-fold and 50-fold, 50-fold and 60-fold, 60-fold and 70-fold, 70-fold and 80-fold, 80-fold and 90-fold, 90-fold and 100-fold, 100-fold and 125-fold, 125-fold and 150-fold, 150-fold and 175-fold, 175-fold and 200-fold, 200-fold and 250-fold, 250-fold and 500-fold, 500-fold and 750-fold, 750-fold and 1,000-fold, 1,000-fold and 2,000-fold, 10-fold and 20,000-fold, 20-fold and 10,000-fold, 50-fold and 5,000-fold, 75-fold and 2,000-fold, 100-fold and 1,000-fold, 200-fold and 500-fold, 10-fold and 100-fold, 100-fold and 200-fold, 200-fold and 500-fold, or 500-fold and 2,000-fold less than the IC₅₀ of an anti-CD20 ADC containing doxorubicin. The ADCs employed when such comparisons are made should include the same antibody and the same linker. Such comparisons should also be made on the same cell types.

Compounds that are at least 40-fold more potent than doxorubicin on CD20-expressing cells under one or more of the foregoing conditions include: DNA minor groove binders, including enediynes and lexitropsins, duocarmycins, taxanes (including paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epithilone A and B, estramustine, cryptophysins, cemadotin, maytansinoids, dolastatins, e.g., auristatin E, dolstatin 10, MMAE (monomethyl Auristatin E), discodermolide, eleutherobin, and mitoxantrone.

In certain specific embodiments, the cytotoxic or cytostatic agent comprises an enediyne moiety. In a specific embodiment, the enediyne moiety is calicheamicin. Enediyne compounds cleave double stranded DNA by generating a diradical via Bergman cyclization.

In other specific embodiments, the cytotoxic or cytostatic agent is auristatin E or a derivative thereof, for example, auristatin EB, monomethyl auristatin E, and auristatin E-FP. The synthesis and structure of auristatin E, also known in the art as dolastatin-10, and its derivatives are described in U.S. patent application Ser. Nos.: 09/845,786 and 10/001,191; in the International Patent Application No.: PCT/US02/13435, in U.S. Pat. Nos: 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, all of which are incorporated by reference in their entireties herein.

In specific embodiments, the drug is a DNA minor groove binding agent. Examples of such compounds and their syntheses are disclosed in U.S. Pat. No.: 6,130,237, which is incorporated by reference in its entirety herein. In certain embodiments, the drug is a CBI compound.

In certain specific embodiments of the invention, the drug is not a polypeptide of greater than 50, 100 or 200 amino acids, for example a toxin. In a specific embodiment of the invention, the drug is not ricin.

In other specific embodiment of the invention, the high potency drug is not one or more of the cytotoxic or cytostatic agents of one of the following non-mutually exclusive classes of agents: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, vinca alkaloids, purine antagonists, and dihydrofolate reductase inhibitors. In more specific embodiments, the high potency drug is not one or more of an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16, VM-26, azothioprine, mycophenolate mofetil, methotrexate, acyclovir, gangcyclovir, zidovudine, vidarabine, ribavarin, azidothymidine, cytidine arabinoside, amantadine, dideoxyuridine, iododeoxyuridine, poscarnet, and trifluridine.

A variety of cytotoxic and cytostatic agents that can be used with the compositions and methods of the present invention are described in U.S. Patent application entitled “Drug Conjugates and their use for treating cancer, an autoimmune disease or an infectious disease”, by Inventors: Peter D. Senter, Svetlana Doronina and Brian E. Toki, submitted on event day herewith, which is incorporated by reference in its entirety herein.

### 5.5.1.1 Dolastatin Drugs

In certain embodiments, the cytotoxic or cytostatic agent is a dolastatin. In more specific embodiments, the dolastatin is of the Auristatin class. In a specific embodiment of the invention, the cytotoxic or cytostatic agent is monomethyl Auristatin E (MMAE; Formula XI). In another specific embodiment of the invention, the cytotoxic or cytostatic agent is Auristatin E-FP (Formula XVI).

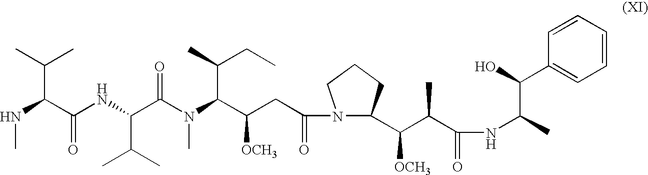

In certain embodiments of the invention, the cytotoxic or cytostatic agent is a dolastatin of formulas XII-XVIII.

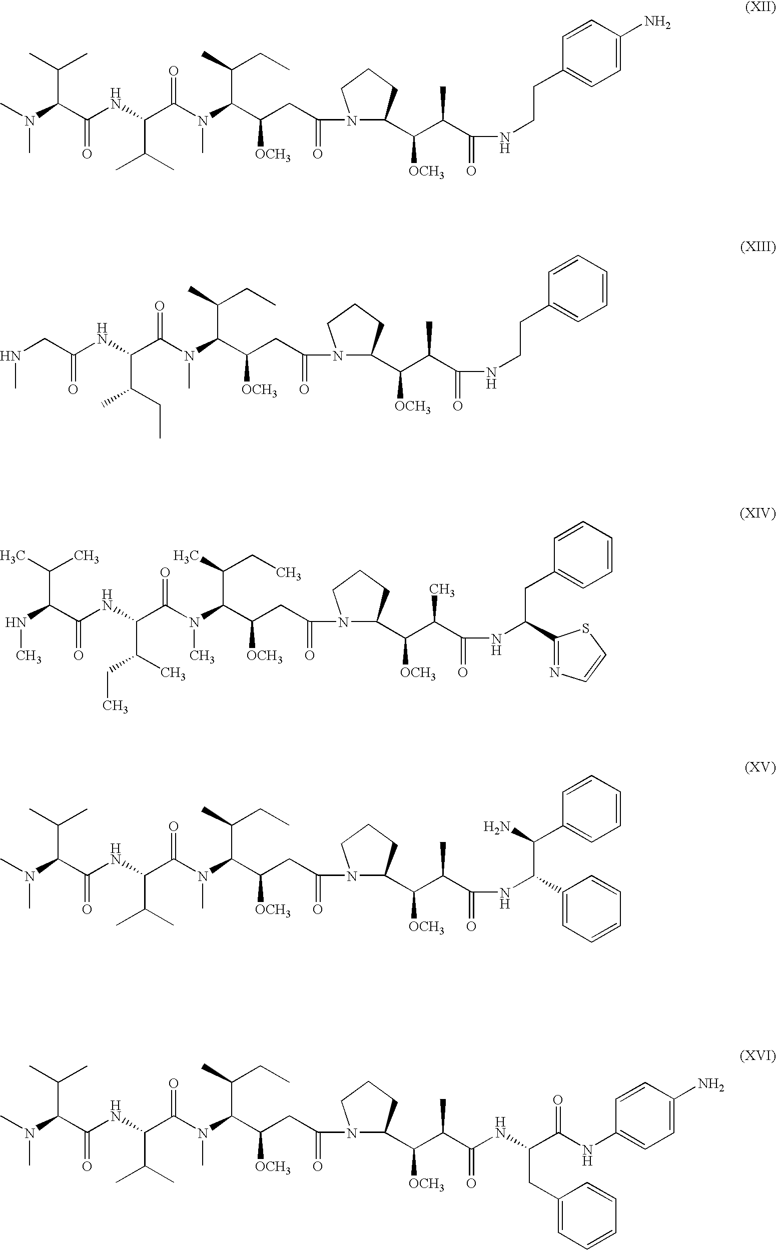

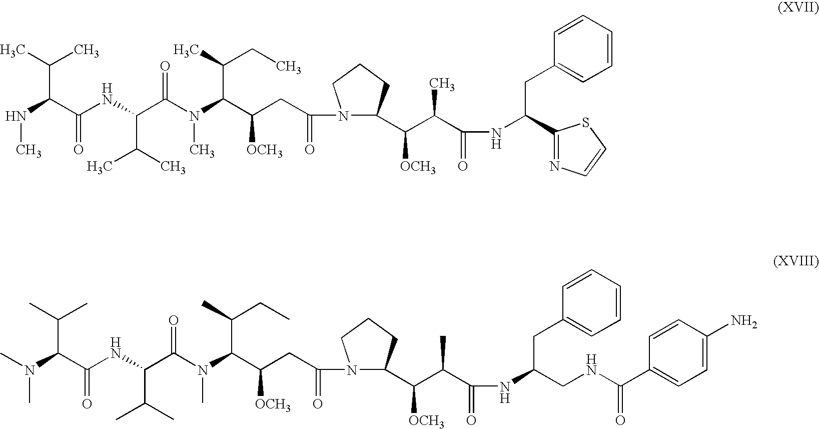

### 5.5.1.2 Assays to Determine Cytotoxicity

In determining whether a given drug is a high potency drug as defined in Section 5.4.1, supra, i.e. whether the drug is suitable for use in an ADC of the invention, the cytotoxicity of the drug, preferably its relative cytotoxicity to doxorubicin, is measured. The cytotoxicity of a drug on CD20-expressing cells, or its relative cytotoxicity to doxorubicin on CD20-expressing cells, can be measured by a variety of different methods known to the skilled artisan. In certain specific embodiments, the relative cytotoxicity of a drug is determined by one of the methods described below.

In certain embodiments of the invention, the sensitivity of cells to the unconjugated drug is measured. Inter alia, the following cell lines can be used to determine the cytotoxicity of an unconjugated drug: Daudi, Ramos, Raji, IM-9, HS-Sultan, ARH-77, HT, RL, DB, or 295R. In a specific embodiment the Karpas cell line is used as a CD20-negative control. In a preferred embodiment, the Raji cell line is used to determine the cytotoxicity of a drug of interest. In a specific embodiment, the cells are exposed to the drug of interest, washed, replated in fresh media and incubated. Several hours before harvest, the cells are treated with alamarBlue™ (BioSource International, Inc.). By monitoring alamarBlue™ reduction spectrophotometrically, cell viability can be determined. Other techniques of determining cell viability are known to the skilled artisan and can be used with the methods of the present invention.

In other embodiments of the invention, sensitivity of cells to ADCs is determined. For example, Daudi, Ramos, Raji, IM-9, HS-Sultan, ARH-77, HT, RL, DB, or 295R cell lines can be used to determine the cytotoxicity of an ADC of interest. In a specific embodiment, the Karpas cell line is used as a CD20-negative control. In a preferred embodiment, the Raji cell line is used to determine the cytotoxicity of an ADC. In a specific embodiment, the cells are exposed to the ADC of interest, washed, replated in fresh media and incubated. Several hours before harvest, the cells are treated with alamarBlue™ (BioSource International, Inc.). By monitoring alamarBlue™ reduction spectrophotometrically, cell viability can be determined. Other techniques of determining cell viability are known to the skilled artisan and can be used with the methods of the present invention. Other staining methods to determine viability of cells include, but are not limited to, Trypan Blue exclusion, Neutral Red staining, Crystal violet inclusion, and ⁵¹Cr release.

In a preferred embodiment of the invention, the IC₅₀ of the cytotoxic agent and doxorubicin is determined by (a) culturing one or more Raji cell populations in the presence of one or more concentrations of the cytotoxic agent for a 72- to 96-hour period; (b) culturing one or more Raji cell populations in the presence of one or more concentrations of doxorubicin for a 72- to 96-hour period, wherein the Raji cell populations are cultured under the same conditions; and (c) identifying a concentration of the cytotoxic agent and doxorubicin, respectively, at which 50% fewer cells in the Raji cell populations, respectively, are viable at the end of the period relative to a Raji cell population cultured under the same conditions in the absence of the cytotoxic agent and doxorubicin such that the concentration of the cytotoxic agent and doxorubicin identified in step (c) is the IC₅₀ of the cytotoxic agent and doxorubicin, respectively. In order to determine the ratio between the IC₅₀ of a cytotoxic agent and the IC₅₀ of doxorubicin, the IC₅₀ of both agents are determined in parallel under the same conditions. The same conditions relate inter alia to the following parameters: approximately the same cell density at the beginning of the assay; the same temperature, culture medium, CO₂ concentration, same period of time of the different incubation and culturing steps. In a preferred embodiment, the IC₅₀ of the cytotoxic agent and doxorubicin are measured in parallel with each other. In another embodiment, a historical control is used to identify the ratio of the IC₅₀ of the cytotoxic agent to doxorubicin.

In a preferred embodiment of the invention, the IC₅₀ of an anti-CD20 antibody-cytotoxic agent conjugate is measured by (a) culturing one or more Raji cell populations in the presence of one or more concentrations of the anti-CD20 antibody-cytotoxic agent conjugate for a 72- to 96-hour period; (b) culturing one or more Raji cell populations in the presence of one or more concentrations of the anti-CD20 antibody-doxorubicin conjugate for a 72- to 96-hour period, wherein the Raji cell populations are cultured under the same conditions; and (c) identifying a concentration of the anti-CD20 antibody-cytotoxic agent conjugate and the anti-CD20 antibody-doxorubicin conjugate, respectively, at which 50% fewer cells in the Raji cell populations, respectively, are viable at the end of the period relative to a Raji cell population cultured under the same conditions in the absence of the anti-CD20 antibody-cytotoxic agent conjugate and the anti-CD20 antibody-doxorubicin conjugate, such that the concentration of the anti-CD20 antibody-cytotoxic agent conjugate and the anti-CD20 antibody-doxorubicin conjugate identified in step (c) is the IC₅₀ of the anti-CD20 antibody-cytotoxic agent conjugate and the anti-CD20 antibody-doxorubicin conjugate, respectively. In order to determine the ratio between the IC₅₀ of an anti-CD20 antibody-cytotoxic agent conjugate and the IC₅₀ of a conjugate of the anti-CD20 antibody and doxorubicin, the IC₅₀ of both agents are determined in parallel under the same conditions. The same conditions relate inter alia to the following parameters: approximately the same cell density at the beginning of the assay; the same temperature, culture medium, CO₂ concentration, same period of time of the different incubation and culturing steps. In a preferred embodiment, the IC₅₀ of the anti-CD20 antibody-cytotoxic agent conjugate and anti-CD20 antibody-doxorubicin conjugate are measured in parallel with each other. In another embodiment, a historical control is used to identify the ratio of the IC₅₀ of the anti-CD20 antibody-cytotoxic agent conjugate to anti-CD20 antibody-doxorubicin conjugate.

In order to compare the cytotoxicity of the drug of interest with the cytotoxicity of doxorubicin, the cytotoxicity of both drugs is measured in parallel under the same conditions. The ratio of the cytoxicity of the drug of interest to the cytoxicity of Doxorubicin can be determined by dividing the IC₅₀ of the drug of interest by the IC₅₀ of Doxorubicin. This ratio can be determined in different cell lines. In a preferred embodiment, the ratio in the cell type for which the lowest differential of cytotoxicity between the drug of interest and Doxorubicin has been measured is used to determine by what factor the drug of interest is more cytotoxic than doxorubicin.

In order to compare the cytotoxicity of the anti-CD20 antibody-drug conjugate of interest with the cytotoxicity of a conjugate of the same anti-CD20 antibody conjugated to doxorubicin, the cytotoxicity of both anti-CD20 ADCs is measured in parallel under the same conditions. The ratio of the cytoxicity of the anti-CD20 antibody-drug conjugate of interest to the cytotoxicity of the anti-CD20 antibody-doxorubicin conjugate can be determined by dividing the IC₅₀ of the anti-CD20 antibody-drug conjugate by the IC₅₀ of the anti-CD20 antibody-doxorubicin conjugate. This ratio can be determined in different cell lines. In a preferred embodiment, the ratio in the cell type for which the lowest differential of cytotoxicity between the anti-CD20 antibody-drug conjugate of interest and the cytotoxicity of the anti-CD20 antibody-doxorubicin conjugate has been measured is used to determine by what factor the anti-CD20 antibody-drug conjugate of interest is more cytotoxic than the anti-CD20 antibody-doxorubicin conjugate. In a preferred embodiment the anti-CD20 antibody-drug conjugate of interest and the anti-CD20 antibody-doxorubicin conjugate comprise the same linker.

In order to determine the cytotoxicity of an ADC of the invention, a CD20 expressing cell line should be used. As a control a CD20 negative cell line can be used. More preferably, the CD20-expressing cell line in the absence of the ADC or in the presence of unconjugated antibody is to be used.

### 5.5.2 Drugs that Enhance Accumulation of an Anti-CD20 Adc Inside a CD20-Expressing Cell

As stated above, in certain embodiments of the invention, an anti-CD20 ADC that is capable of accumulating inside a CD20-expressing cell at a rate that results in a cytotoxic or cytostatic effect is engineered by conjugating an anti-CD20 antibody to a drug that enhances accumulation of the conjugate inside the cell. In certain embodiments, accumulation of the anti-CD20 antibody-drug conjugate inside the cell is enhanced relative to accumulation inside the cell of a comparable anti-CD20-doxorubicin conjugate. In other embodiment, accumulation inside the cell is enhanced relative to accumulation inside the cell of an unconjugated antibody. The rate of accumulation inside a CD20-expressing cell is the net effect of internalization of the conjugate into the cell and export of the conjugate out of the cell.

In certain embodiments of the invention, the drug that promotes accumulation inside the cell is an anti-tubulin agent. Anti-tubulin agents are a well established class of cancer therapy compounds. Examples of anti-tubulin agents include, but are not limited to, taxanes (e.g., Taxol® (paclitaxel), docetaxel), T67 (Tularik), vincas, and auristatins (e.g., auristatin E, auristatin EB, monomethyl auristatin E, auristatin E FP). Antitubulin agents included in this class are also: vinca alkaloids, including vincristine and vinblastine, vindesine and vinorelbine; taxanes such as paclitaxel and docetaxel and baccatin derivatives, epithilone A and B, nocodazole, colchicine and colcimid, estramustine, cryptophysins, cemadotin, maytansinoids, combretastatins, dolastatins, discodermolide and eleutherobin.

In certain embodiments, the rate of accumulation of an anti-CD20 antibody-cytotoxic agent conjugate inside a CD20-expressing cell is at least 1.5-fold, 2-fold, 5-fold, 10-fold, 20-fold, 50-fold, 200-fold, 500-fold, or 1000-fold greater than the rate of accumulation inside a CD20-expressing cell of an anti-CD20 antibody in unconjugated form. In certain embodiments, the rate of accumulation of an anti-CD20 antibody-cytotoxic agent conjugate inside a CD20-expressing cell is at most 2-fold, 5-fold, 10-fold, 20-fold, 50-fold, 200-fold, 500-fold, or 1000-fold greater than the rate of accumulation inside a CD20-expressing cell of an anti-CD20 antibody in unconjugated form. In certain embodiments, the rate of accumulation of an anti-CD20 antibody-cytotoxic agent conjugate inside a CD20-expressing cell is between 20-fold and 5,000-fold, 50-fold and 2,500-fold, 100-fold and 1,000-fold, 100-fold and 500-fold, 1.5-fold and 2-fold, 2-fold and 5-fold, 5-fold and 20-fold, 20-fold and 50-fold, 50-fold and 500-fold, 500-fold and 5,000-fold, 1.5-fold and 5-fold, 5-fold and 50-fold, 50-fold and 5,000-fold, 1.5-fold and 5,000-fold, 2-fold and 5,000-fold, 5-fold and 200-fold, 10-fold and 100-fold, or 25-fold and 75-fold greater than the rate of accumulation inside a CD20-expressing cell of an anti-CD20 antibody in unconjugated form.

In certain other embodiments, the rate of accumulation of an anti-CD20 antibody-cytotoxic agent conjugate inside a CD20-expressing cell is at least 1.5-fold, 2-fold, 5-fold, 10-fold, 20-fold, 50-fold, 200-fold, 500-fold, or 1000-fold greater than the rate of accumulation inside a CD20-expressing cell of a conjugate of the anti-CD20 antibody and doxorubicin. In certain embodiments, the rate of accumulation of an anti-CD20 antibody-cytotoxic agent conjugate inside a CD20-expressing cell is at most 2-fold, 5-fold, 10-fold, 20-fold, 50-fold, 200-fold, 500-fold, or 1,000-fold greater than the rate of accumulation inside a CD20-expressing cell of a conjugate of the anti-CD20 antibody and doxorubicin. In certain embodiments, the rate of accumulation of an anti-CD20 antibody-cytotoxic agent conjugate inside a CD20-expressing cell is between 20-fold and 5,000-fold, 50-fold and 2,500-fold, 100-fold and 1,000-fold, 100-fold and 500-fold, 1.5-fold and 2-fold, 2-fold and 5-fold, 5-fold and 20-fold, 20-fold and 50-fold, 50-fold and 500-fold, 500-fold and 5,000-fold, 1.5-fold and 5-fold, 5-fold and 50-fold, 50-fold and 5,000-fold, 1.5-fold and 5,000-fold, 2-fold and 5,00-fold, 5-fold and 200-fold, 10-fold and 100-fold, or 25-fold and 75-fold greater than the rate of accumulation inside a CD20-expressing cell of a conjugate of the anti-CD20 antibody and doxorubicin.

In a specific embodiment, the rate of accumulation inside a CD20 expressing cell of an anti-CD20 antibody ADC is measured by incubating a CD20 expressing cell with isotopically labeled anti-CD20 antibody ADC under conditions conducive to accumulation of the ADC inside the cell. The isotope labeling can be on the antibody, the linker, or the drug moiety of the anti-CD20 ADC, but is preferably on the antibody so that the rate can be compared to that of a similarly labeled, unconjugated antibody or an antibody conjugated to doxorubicin. Subsequent to the incubation step, any anti-CD20 antibody ADC that is bound to the cell surface is removed by acidic washing steps. Subsequently, the radioactivity inside the cells is measured by any method known to the skilled artisan, such as by placing the cells into a scintillation counter. The amount of radioactivity measured is proportional to the anti-CD20 antibody ADC accumulated inside the CD20 expressing cells.

To determine the ratio of the rates of accumulation inside a CD20 expressing cells between an anti-CD20 antibody ADC and a conjugate of the anti-CD20 antibody and doxorubin or the unconjugated antibody, the respective rates are measured under the same conditions in parallel. The same conditions relate inter alia to the following parameters: approximately the same cell density at the beginning of the assay, the same number of cells being assayed, the same temperature, culture medium, CO₂ concentration, same period of time of the different incubation and culturing steps.

Determining the differential rate of accumulation does not require measuring and comparing absolute rates of accumulation. Rather, the relative amounts of radioactivity taken up by the CD20-expressing cells in a given time period under similar conditions can be used as an indicator of the relative rates of accumulation of the anti-CD20 ADC of the invention, the unconjugated antibody, or the anti-CD20 antibody-doxorubicin conjugate. Any CD20-expressing cell can be used for such measurements, but most preferably a cell line such as, but not limited to Raji, Ramos, Daudi, IM-9, HS-Sultan, ARH-77, HT, RL, DB, or 295R cell line, is used.

In other embodiments, the antibody is labeled with a fluorescent label rather than a radioisotope. The relative rate of accumulation of the fluorescent label, for example as measured by fluorometry, can be used to determine the relative rates of ADC versus unconjugated/doxorubicin-conjugated antibody accumulation inside the cells. As discussed above for the radioisotope accumulation assay, ADC or antibody bound to the surface of the CD20-expressing cells is removed from the cells prior to measuring the amount of fluorescent signal that has accumulated inside the cell, for example by using one or more acid washes.

In certain embodiments, immunocytochemistry is used to determine the rate of accumulation of the ADC in a nonperipheral region of a CD20 expressing cell. In these embodiments, CD20-expressing cells are cultured with an anti-CD20 ADC, and under the same conditions the CD20-expressing cells are cultured with the anti-CD20 antibody in unconjugated form. In a preferred embodiment, the different cultures are done in parallel. In other embodiments, a historical control is used. The same conditions relate inter alia to the following parameters: approximately the same cell density at the beginning of the assay, approximately the same number of cells being assayed, the same temperature, culture medium, CO₂ concentration, same period of time of the different incubation and culturing steps. In specific embodiments, the CD20-expressing cells are cultured with the anti-CD20 ADC and the anti-CD20 antibody in unconjugated form for at least 10 minutes, 30 minutes, 1 hour, 4 hours, 12 hours, or 24 hours. In specific embodiments, the CD20-expressing cells are cultured with the anti-CD20 ADC and the anti-CD20 antibody in unconjugated form for at most 10 minutes, 30 minutes, 1 hour, 4 hours, 12 hours, or 24 hours. In a specific embodiment, a timecourse is taken with timepoints at 10 minutes, 30 minutes, 1 hour, 4 hours, 12 hours, and 24 hours. In specific embodiments, the cells are cultured at 37° C. Subsequent to the culturing step, the cells are fixed, permeabilized and stained with an anti-human IgG specific antibody (e.g., an anti-human IgG Fcγ specific antibody) labeled with a fluorescent label. The localization of the fluorescence signal is then determined by confocal fluorescence microscopy. In certain embodiments of the invention, the focal plane is an equatorial section of the cell. In this section, peripheral staining can be distinguished from non-peripheral staining, i.e., staining in the interior of the cell. In specific embodiments, a non-peripheral region of the cell is a region of the cell that is not associated with the cytoplasmic membrane. In specific embodiments, the CD20-expressing cells are double stained to detect the anti-CD20 ADC or the unconjugated anti-CD20 antibody, respectively, and a marker of the cell periphery. In a specific embodiment, Spectrin is used as a marker of the cell periphery. In another specific embodiment Wheat Germ Agglutinin (WGA) is used as a marker of the cell periphery. In specific embodiments, accumulation of the anti-CD20 ADC in a non-peripheral region inside the CD20-expressing cell as determined by immunocytochemistry can be compared to accumulation of the unconjugated anti-CD20 antibody statistically. The percentage of cells in a population of CD20-expressing cells with a detectable amount of the anti-CD20 ADC in a non-peripheral region of the cell is determined and compared to the percentage of cells in a population of CD20-expressing cells with a detectable amount of the unconjugated anti-CD20 antibody in a non-peripheral region. The total number of cells in the populations assayed should be comparable to each other. In specific embodiments, the population size assayed is at least 10, 50, 100, 500 or 1000 cells. In certain embodiments, at least 1.5-fold, 2-fold, 5-fold, 20-fold, 50-fold, 500-fold, or 5,000-fold more CD20-expressing cells show a detectable amount of the anti-CD20 ADC in a non-peripheral region than CD20-expressing cells show a detectable amount of the unconjugated anti-CD20 antibody in a non-peripheral region. In certain embodiments, at most 1.5-fold, 2-fold, 5-fold, 20-fold, 50-fold, 500-fold, or 5,000-fold more CD20-expressing cells show a detectable amount of the anti-CD20 ADC in a non-peripheral region than CD20-expressing cells show a detectable amount of the unconjugated anti-CD20 antibody in a non-peripheral region. In certain embodiments, between 20-fold and 5,000-fold, 50-fold and 2,500-fold, 100-fold and 1,000-fold, 100-fold and 500-fold, 1.5-fold and 2-fold, 2-fold and 5-fold, 5-fold and 20-fold, 20-fold and 50-fold, 50-fold and 500-fold, 500-fold and 5,000-fold, 1.5-fold and 5-fold, 5-fold and 50-fold, 50-fold and 5,000-fold, 1.5-fold and 5,000-fold, 2-fold and 5,00-fold, 5-fold and 200-fold, 10-fold and 100-fold, or 25-fold and 75-fold more CD20-expressing cells show a detectable amount of the anti-CD20 ADC in a non-peripheral region than CD20-expressing cells show a detectable amount of the unconjugated anti-CD20 antibody in a non-peripheral region.

In specific embodiments, accumulation of the anti-CD20 ADC in a non-peripheral region inside the CD20-expressing cell can be compared to accumulation of the unconjugated anti-CD20 antibody by comparing the intensity of the staining in the immunocytochemistry in a nonperipheral region of the CD20-expressing cell. In certain embodiments, accumulation of the anti-CD20 ADC in a non-peripheral region of the majority of the CD20-expressing cells assayed is at least 1.5-fold, 2-fold, 5-fold, 20-fold, 50-fold, 500-fold, or 5,000-fold higher than the average accumulation of the unconjugated anti-CD20 antibody in a non-peripheral region of the CD20-expressing cell. In certain embodiments, accumulation of the anti-CD20 ADC in a non-peripheral region of the majority of the CD20-expressing cells assayed is at most 1.5-fold, 2-fold, 5-fold, 20-fold, 50-fold, 500-fold, or 5,000-fold higher than the average accumulation of the unconjugated anti-CD20 antibody in a non-peripheral region of the CD20-expressing cell. In certain embodiments, accumulation of the anti-CD20 ADC in a non-peripheral region of the majority of the CD20-expressing cells assayed is between 20-fold and 5,000-fold, 50-fold and 2,500-fold, 100-fold and 1,000-fold, 100-fold and 500-fold, 1.5-fold and 2-fold, 2-fold and 5-fold, 5-fold and 20-fold, 20-fold and 50-fold, 50-fold and 500-fold, 500-fold and 5,000-fold, 1.5-fold and 5-fold, 5-fold and 50-fold, 50-fold and 5,000-fold, 1.5-fold and 5,000-fold, 2-fold and 5,00-fold, 5-fold and 200-fold, 10-fold and 100-fold, or 25-fold and 75-fold higher than the average accumulation of the unconjugated anti-CD20 antibody in a non-peripheral region of the CD20-expressing cell. In specific embodiments, the majority of cells is at least 60%, 70%, 80%, 90%, or 98% of the cells assayed. In specific embodiments, the majority of cells is at most 70%, 80%, 90%, or 98% of the cells assayed.

In certain embodiments, CD20-expressing cells are cultured with an anti-CD20 ADC, and under the same conditions the CD20-expressing cells are cultured with anti-CD20 antibody-doxorubicin conjugate. In preferred embodiments, the CD20 antibody and the linker of the conjugates are the same. In a preferred embodiment, the different cultures are done in parallel. In other embodiments, a historical control is used. The same conditions relate inter alia to the following parameters: approximately the same cell density at the beginning of the assay, approximately same number of cells assayed, the same temperature, culture medium, CO₂ concentration, same period of time of the different incubation and culturing steps. In specific embodiments, the CD20-expressing cells are cultured with the anti-CD20 ADC and the anti-CD20 antibody-doxorubicin conjugate, respectively, for at least 10 minutes, 30 minutes, 1 hour, 4 hours, 12 hours, or 24 hours. In specific embodiments, the CD20-expressing cells are cultured with the anti-CD20 ADC and the anti-CD20 antibody-doxorubicin conjugate, respectively, for at most 10 minutes, 30 minutes, 1 hour, 4 hours, 12 hours, or 24 hours. In a specific embodiment, a timecourse is taken with timepoints at 10 minutes, 30 minutes, 1 hour, 4 hours, 12 hours, and 24 hours. In specific embodiments, the cells are cultured at 37° C. Subsequent to the culturing step, the cells are fixed, permeabilized and stained with an anti-human IgG Fcγ specific antibody labeled with a fluorescent label. The localization of the fluorescence signal is then determined by confocal fluorescence microscopy. In certain embodiments of the invention, the focal plane is an equatorial section of the cell. In this section, peripheral staining can be distinguished from non-peripheral staining, i.e., staining in the interior of the cell. In specific embodiments, the CD20-expressing cells are double stained to detect the anti-CD20 ADC or the anti-CD20 antibody-doxorubicin conjugate, respectively, and a marker of the cell periphery. In a specific embodiment, Spectrin is used as a marker of the cell periphery. In another specific embodiment Wheat Germ Agglutinin (WGA) is used as a marker of the cell periphery. In specific embodiments, accumulation of the anti-CD20 ADC in a non-peripheral region inside the CD20-expressing cell can be compared to accumulation of the anti-CD20 antibody-doxorubicin conjugate statistically. The percentage of cells in a population of CD20-expressing cells with a detectable amount of the anti-CD20 ADC in a non-peripheral region of the cell is determined and compared to the percentage of cells in a population of CD20-expressing cells with a detectable amount of the anti-CD20 antibody-doxorubicin conjugate in a non-peripheral region. The total number of cells in the populations assayed should be comparable to each other. In specific embodiments, the population size assayed is at least 10, 50, 100, 500 or 1000 cells. In certain embodiments, at least 1.5-fold, 2-fold, 5-fold, 20-fold, 50-fold, 500-fold, or 5,000-fold more CD20-expressing cells show a detectable amount of the anti-CD20 ADC in a non-peripheral region than CD20-expressing cells show a detectable amount of the anti-CD20 antibody-doxorubicin conjugate in a non-peripheral region. In certain embodiments, at most 1.5-fold, 2-fold, 5-fold, 20-fold, 50-fold, 500-fold, or 5,000-fold more CD20-expressing cells show a detectable amount of the anti-CD20 ADC in a non-peripheral region than CD20-expressing cells show a detectable amount of the anti-CD20 antibody-doxorubicin conjugate in a non-peripheral region. In certain embodiments, between 20-fold and 5,000-fold, 50-fold and 2,500-fold, 100-fold and 1,000-fold, 100-fold and 500-fold, 1.5-fold and 2-fold, 2-fold and 5-fold, 5-fold and 20-fold, 20-fold and 50-fold, 50-fold and 500-fold, 500-fold and 5,000-fold, 1.5-fold and 5-fold, 5-fold and 50-fold, 50-fold and 5,000-fold, 1.5-fold and 5,000-fold, 2-fold and 5,00-fold, 5-fold and 200-fold, 10-fold and 100-fold, or 25-fold and 75-fold more CD20-expressing cells show a detectable amount of the anti-CD20 ADC in a non-peripheral region than CD20-expressing cells show a detectable amount of the anti-CD20 antibody-doxorubicin conjugate in a non-peripheral region.

In specific embodiments, accumulation of the anti-CD20 ADC in a non-peripheral region inside the CD20-expressing cell can be compared to accumulation of the anti-CD20 antibody-doxorubicin conjugate by comparing the intensity of the staining obtained by immunocytochemistry (see above) inside the CD20-expressing cell. In certain embodiments, accumulation of the anti-CD20 ADC in a non-peripheral region of the majority of the CD20-expressing cells assayed is at least 1.5-fold, 2-fold, 5-fold, 20-fold, 50-fold, 500-fold, or 5,000-fold higher than the average accumulation of the anti-CD20 antibody-doxorubicin conjugate in a non-peripheral region of the CD20-expressing cell. In certain embodiments, accumulation of the anti-CD20 ADC in a non-peripheral region of the majority of the CD20-expressing cells assayed is at most 1.5-fold, 2-fold, 5-fold, 20-fold, 50-fold, 500-fold, or 5,000-fold higher than the average accumulation of the anti-CD20 antibody-doxorubicin conjugate in a non-peripheral region of the CD20-expressing cell. In certain embodiments, accumulation of the anti-CD20 ADC in a non-peripheral region of the majority of the CD20-expressing cells assayed is between 20-fold and 5,000-fold, 50-fold and 2,500-fold, 100-fold and 1,000-fold, 100-fold and 500-fold, 1.5-fold and 2-fold, 2-fold and 5-fold, 5-fold and 20-fold, 20-fold and 50-fold, 50-fold and 500-fold, 500-fold and 5,000-fold, 1.5-fold and 5-fold, 5-fold and 50-fold, 50-fold and 5,000-fold, 1.5-fold and 5,000-fold, 2-fold and 5,00-fold, 5-fold and 200-fold, 10-fold and 100-fold, or 25-fold and 75-fold higher than the average accumulation of the anti-CD20 antibody-doxorubicin conjugate in a non-peripheral region of the CD20-expressing cell. In specific embodiments, the majority of cells is at least 60%, 70%, 80%, 90%, or 98% of the cells assayed. In specific embodiments, the majority of cells is at most 70%, 80%, 90%, or 98% of the cells assayed.

In a specific embodiment, the drug is a maytansinoid, a group of anti-tubulin agents. In a more specific embodiment, the drug is maytansine. Further, in a specific embodiment, the cytotoxic or cytostatic agent is DM-1 (ImmunoGen, Inc.; see also Chari et al, 1992, Cancer Res 52:127-131). Maytansine, a natural product, inhibits tubulin polymerization resulting in a mitotic block and cell death. Thus, the mechanism of action of maytansine appears to be similar to that of vincristine and vinblastine. Maytansine, however, is about 200 to 1,000-fold more cytotoxic in vitro than these Vinca alkaloids.

In another specific embodiment, the drug is auristatin E derivative, auristatin E FP (“AEFP”).

### 5.6 Formation of Anti-CD20 Antibody-Drug Conjugates

The generation of anti-CD20 antibody drug conjugates (ADCs) can be accomplished by any technique known to the skilled artisan. Briefly, the anti-CD20 ADCs comprise an anti-CD20 antibody, a drug, and a linker that joins the drug and the antibody. A number of different reactions are available for covalent attachment of drugs to antibodies. This often accomplished by reaction of the amino acid residues of the antibody molecule, including the amine groups of lysine, the free carboxylic acid groups of glutamic and aspartic acid, the sulfhydryl groups of cysteine and the various moieties of the aromatic amino acids. One of the most commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of the antibody molecule. Also available for attachment of drugs to antibodies is the Schiff base reaction. This method involves the periodate oxidation of a drug that contains glycol or hydroxy groups, thus forming an aldehyde which is then reacted with the antibody molecule. Attachment occurs via formation of a Schiff base with amino groups of the antibody molecule. Isothiocyanates can also be used as coupling agents for covalently attaching drugs to antibodies. Other techniques known to the skilled artisan and within the scope of the present invention. Non-limiting examples of such techniques are described in, e.g., U.S. Pat. Nos. 5,665,358, 5,643,573, and 5,556,623, which are incorporated by reference in their entireties herein.

In certain embodiments, an intermediate, which is the precursor of the linker, is reacted with the drug under appropriate conditions. In certain embodiments, reactive groups are used on the drug and/or the intermediate. The product of the reaction between the drug and the intermediate, or the derivatized drug, is subsequently reacted with the anti-CD20 antibody under appropriate conditions. Care should be taken to maintain the stability of the antibody under the conditions chosen for the reaction between the derivatized drug and the antibody.

### 5.7 Therapeutic/Prophylactic Administration and Compositions

The invention provides methods of treating and preventing proliferative disorders of cells that express CD20, for example CD20-expressing cancers and B-cell associated immune disorders.

The outcome of the present therapeutic and prophylactic methods is to at least produce in a patient a healthful benefit, which includes but is not limited to: prolonging the lifespan of a patient, prolonging the onset of symptoms of cancer or an immune disorder, and/or alleviating a symptom of cancer or the disorder after onset of a symptom. Where the CD20-associated disorder is cancer of CD20-expressing cells, such a healthful benefit can result in delaying tumor growth and/or promoting tumor regression. Where the CD20-associated disorder is an immune disorder, such a healthful benefit can result inhibiting disease progression and/or reducing disease symptoms.

As used herein, the term “prevention” refers to administration of an ADC of the invention to the patient before the onset of symptoms or molecular indications of the cancer or immune disorder of interest, for example to an individual with a predisposition or at a high risk of acquiring the cancer or immune disorder. In contrast, the term “treatment” refers to administration of an ADC of the present invention to the patient after the onset of symptoms or molecular indications of the cancer or immune disorder at any clinical stage.

In a preferred aspect, the anti-CD20 antibody-drug conjugate is substantially purified (e.g., substantially free from substances that limit its effect or produce undesired side-effects). In certain specific embodiments, the anti-CD20 antibody-drug conjugate is 40% pure, more preferably about 50% pure, and most preferably about 60% pure. In certain specific embodiments, the anti-CD20 antibody-drug conjugate is approximately 60-65%, 65-70%, 70-75%, 75-80%, 80-85%, 85-90%, 90-95%, or 95-98% pure. In another specific embodiment, the anti-CD20 antibody-drug conjugate is approximately 99% pure.

The subject is preferably an animal, including but not limited to animals such as cows, pigs, horses, chickens, cats, dogs, etc., and is preferably a mammal, and most preferably human.

Formulations and methods of administration that can be employed are described below; additional appropriate formulations and routes of administration can be selected from among those described herein below.

Various delivery systems are known and can be used to administer an anti-CD20 antibody in accordance with the methods of the present invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432). Methods of introduction include but are not limited to intradermal, intramuscular, intraperitoneal, intravenous, subcutaneous, intranasal, epidural, and oral routes. The anti-CD20 antibody-drug conjugates may be also administered by any convenient route, for example by infusion or bolus injection, by absorption through epithelial or mucocutaneous linings (e.g., oral mucosa, rectal and intestinal mucosa, etc.) and may be administered together with other biologically active agents, including but not limited to an immunogenic molecule. Administration can be systemic or local.

In a specific embodiment, it may be desirable to administer the anti-CD20 antibody-drug conjugate by injection, by means of a catheter, by means of a suppository, or by means of an implant, said implant being of a porous, non-porous, or gelatinous material, including a membrane, such as a sialastic membrane, or a fiber. Preferably, when administering an anti-CD20 antibody-drug conjugate, care must be taken to use materials to which the anti-CD20 antibody-drug conjugate does not absorb.

In another embodiment, the anti-CD20 antibody-drug conjugate can be delivered in a vesicle, in particular a liposome (see Langer, 1990, Science 249:1527-1533; Treat et al., 1989, in Liposomes in the Therapy of Infectious Disease and Cancer, Lopez-Berestein and Fidler (eds.), Liss, New York, pp. 353-365; Lopez-Berestein, ibid., pp. 317-327; see generally, ibid.)

In yet another embodiment, the anti-CD20 antibody-drug conjugate can be delivered in a controlled release system. In one embodiment, a pump may be used (see Langer, supra; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, New York; Ranger and Peppas, 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

Other controlled release systems are discussed in the review by Langer, 1990, Science 249:1527-1533.

Pharmaceutical compositions comprising an amount of anti-CD20 antibody-drug conjugate effective to treat a CD20-expressing cancer or an immune disorder involving CD20-expressing cells further comprise a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the anti-CD20 antibody-drug conjugate is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate) lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicles before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Examples of suitable pharmaceutical carriers are described in “Remington's Pharmaceutical Sciences” by E. W. Martin. Such compositions will contain a therapeutically effective amount of the anti-CD20 antibody-drug conjugate, preferably in purified form, together with a suitable amount of carrier so as to provide the form for proper administration to the patient. The formulation should suit the mode of administration.

Preparations for oral administration may be suitably formulated to give controlled release of the active compound.

For buccal administration the compositions may take the form of tablets or lozenges formulated in conventional manner.

For administration by inhalation, the anti-CD20 antibodies are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The anti-CD20 antibodies may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The anti-CD20 antibodies may also be formulated in rectal compositions such as suppositories or retention enemas, e.g., containing conventional suppository bases such as cocoa butter or other glycerides.

In addition to the formulations described previously, the anti-CD20 antibodies may also be formulated as a depot preparation. Such long acting formulations may be administered by implantation (for example subcutaneously or intramuscularly) or by intramuscular injection. Thus, for example, the proteins may be formulated with suitable polymeric or hydrophobic materials (for example as an emulsion in an acceptable oil) or ion exchange resins, or as sparingly soluble derivatives, for example, as a sparingly soluble salt.

In a preferred embodiment, the pharmaceutical composition is formulated in accordance with routine procedures as a pharmaceutical composition adapted for intravenous administration to human beings. Typically, compositions for intravenous administration are solutions in sterile isotonic aqueous buffer. Where necessary, the pharmaceutical composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent, i.e., the anti-CD20 antibody-drug conjugate. Where the pharmaceutical of the invention is to be administered by infusion, it can be dispensed with an infusion bottle containing sterile pharmaceutical grade water or saline. Where the pharmaceutical composition comprising anti-CD20 antibody-drug conjugate is administered by injection, an ampoule of sterile water for injection or saline can be provided so that the ingredients may be mixed prior to administration.

The anti-CD20 antibody-drug conjugate compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration preferably for administration to a human.

### 5.8 Target Cancers

The compositions and methods of the present invention are useful for treating or preventing a CD20-expressing cancer. Treatment or prevention of a CD20-expressing cancer, according to the methods of the present invention, is achieved by administering to a patient in need of such treatment or prevention an anti-CD20 conjugate of the invention. The methods of the present invention are useful for the treatment of different subtypes of indolent Non-Hodgkin's Lymphoma (indolent NHLs). Among the indolent NHLs to be treated by the methods of the invention are: follicular NHLs, small lymphocytic lymphomas, chronic lymphocytic leukemias, lymphoplasmacytic NHLs, and marginal zone NHLs. Other cancers involving CD20 expressing cells are cancers of the B-cell lineage and multiple myeloma. Other cancers that can be treated using the methods of the invention are, inter alia, hairy cell leukemia, B cell prolymphocytic leukemia, and CD20-positive Acute lymphocytic leukemia.

### 5.9 Target Immune Disorders

The methods of the present invention are useful for treating or preventing an immune disorder, wherein the immune disorder is characterized by non-neoplastic inappropriate proliferation of CD20-expressing cells of the immune system. Treatment or prevention of an immune disorder, according to the methods of the present invention, is achieved by administering to a patient in need of such treatment or prevention an anti-CD20 conjugate of the invention.

Examples of diseases that can be treated or prevented by the methods of the present invention include, but are not limited to, rheumatoid arthritis, multiple sclerosis, endocrine ophthalmopathy, uveoretinitis, systemic lupus erythematosus, myasthenia gravis, Grave's disease, glomerulonephritis, autoimmune hepatological disorder, autoimmune inflammatory bowel disease, anaphylaxis, allergic reaction, Sjogren's syndrome, juvenile onset (Type I) diabetes mellitus, primary biliary cirrhosis, Wegener's granulomatosis, fibromyalgia, inflammatory bowel disease, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, presenile dementia, demyelinating diseases, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia areata, pemphigoid, scleroderma, progressive systemic sclerosis, CREST syndrome (calcinosis, Raynaud's phenomenon, esophageal dysmotility, sclerodactyly, and telangiectasia), adult onset diabetes mellitus (Type II diabetes), male and female autoimmune infertility, ankylosing spondolytis, ulcerative colitis, Crohn's disease, mixed connective tissue disease, polyarteritis nedosa, systemic necrotizing vasculitis, juvenile onset rheumatoid arthritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic encephalomyelitis, toxic epidermal necrolysis, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Eaton-Lambert syndrome, relapsing polychondritis, cryoglobulinemia, Waldenstrom's macroglobulemia, Epstein-Barr virus infection, mumps, Evan's syndrome, hairy cell leukemia, B cell prolymphocytic leukemia, and CD20-positive Acute lymphocytic leukemia, and autoimmune gonadal failure.

In certain embodiments, the immune disorder is a lymphocytosis. In certain specific embodiments, the immune disorder is a Primary lymphocytosis, which includes monoclonal B cell lymphocytosis (benign monoclonal gammopathy and monoclonal gammopathy of undetermined significance; MGUS). These may later in life develop into progressive neoplastic lymphoproliferative diseases but early on are considered immune disorders and not cancer. In other specific embodiments, the lymphocytosis is a Secondary (reactive) lymphocytosis including infectious mononucleosis, acute infection lymphocytosis, Bordetella Pertusis infection, stress-induced lymphocytosis, and persistent lymphocytosis (including autoimmune diseases, chronic inflammatory diseases and hypersensitivity reactions).

In a preferred embodiment, the diseases that can be treated include, but are not limited to, rheumatoid arthritis, multiple sclerosis, endocrine ophthalmopathy, systemic lupus erythematosus, myasthenia gravis, Grave's disease, glomerulonephritis, anaphylaxis, allergic reaction, Sjogren's syndrome, juvenile onset (Type I) diabetes mellitus, primary biliary cirrhosis, Wegener's granulomatosis, inflammatory bowel disease, polymyositis, dermatomyositis, Schmidt's syndrome, Addison's disease, adrenalitis, thyroiditis, Hashimoto's thyroiditis, autoimmune thyroid disease, pernicious anemia, chronic hepatitis, lupoid hepatitis, atherosclerosis, demyelinating diseases, subacute cutaneous lupus erythematosus, hypoparathyroidism, autoimmune thrombocytopenia, idiopathic thrombocytopenic purpura, hemolytic anemia, pemphigus vulgaris, pemphigus, dermatitis herpetiformis, alopecia areata, pemphigoid, scleroderma, progressive systemic sclerosis, CREST syndrome (calcinosis, Raynaud's phenomenon, esophageal dysmotility, sclerodactyly, and telangiectasia), adult onset diabetes mellitus (Type II diabetes), ulcerative colitis, Crohn's disease, mixed connective tissue disease, polyarteritis nedosa, systemic necrotizing vasculitis, juvenile onset rheumatoid arthritis, atopic rhinitis, Goodpasture's syndrome, asthma, anti-phospholipid syndrome, farmer's lung, erythema multiforme, autoimmune chronic active hepatitis, bird-fancier's lung, allergic encephalomyelitis, toxic epidermal necrolysis, alveolitis, allergic alveolitis, fibrosing alveolitis, erythema nodosum, transfusion reaction, Caplan's syndrome, erythroblastosis fetalis, Felty's syndrome, IgA nephropathy, Henoch-Schonlein purpura, graft versus host disease, transplantation rejection, relapsing polychondritis, cryoglobulinemia, Waldenstrom's macroglobulemia, Epstein-Barr virus infection, hairy cell leukemia, B cell prolymphocytic leukemia, and CD20-positive Acute lymphocytic leukemia, and autoimmune gonadal failure.

Accordingly, the methods of the present invention encompass treatment of disorders of B-lymphocytes (e.g., systemic lupus erythematosus, Goodpasture's syndrome, rheumatoid arthritis, and type I diabetes), TH1-lymphocytes (e.g., rheumatoid arthritis, multiple sclerosis, psoriasis, Sjorgren's syndrome, Hashimoto's thyroiditis, Grave's disease, primary biliary cirrhosis, Wegener's granulomatosis, or tuberculosis), and TH2-lymphocytes (e.g., atopic dermatitis, atopic asthma, rhinoconjunctivitis, allergic rhinitis, Omenn's syndrome, systemic sclerosis, or graft versus host disease).

An alternative way of classifying immune disease states is by the underlying biological mechanism. The present invention is directed to treatment and prevention of immune diseases arising by any of the following mechanisms, which are classified into four types:

Anaphylactic reactions. These reactions are mediated by IgE antibodies which bind to receptors on mast cells. When cross-linking occurs with antigens, the IgE antibodies stimulate the mast cells to release a number of pharmacologically active substances that can cause the symptoms characteristic of anaphylaxis. These reactions to antigenic challenge are immediate and potentially life-threatening. Examples of anaphylactic responses include, but are not limited to, allergic rhinitis, gastrointestinal allergy, atopic dermatitis, bronchial asthma and equine heaves and laminitis.

Cytotoxic (cytolytic) reactions. These cell surface reactions result from an interaction of antigen with IgM and/or IgG which activates the complement cascade, leading to the destruction of the cell. Examples of cytolytic reactions include, but are not limited to, leukocytopenia, hemolytic disease of newborn and Goodpasture's disease. Autoimmune disorders that involve cytotoxic/cytolytic reactions are hemolytic anemia, thrombocytopenia and thyroiditis.

Immune complex reactions. Immune complex reactions occur when large complexes of antigen and IgG or IgM accumulate in the circulation or in tissue, fixing complement. Granulocytes are attracted to the site of complement fixation and release damaging lytic enzymes from their granules. An example of this type of reaction is serum sickness. Autoimmune disorders that involve immune complex reactions include systemic lupus erythrematosus, chronic glomerulonephritis and rheumatoid arthritis.

Cell-mediated immunity (CMI) reaction, or delayed-type hypersensitivity (DTH). In contrast to the first three types of immune responses, this hypersensitivity response is mediated by T lymphocytes rather than antibodies produced by B lymphocytes. Activated T lymphocytes release cytokines which can result in the accumulation and activation of macrophages, K cells and NK cells, which cause local tissue damage. This reaction can occur 1-2 days after antigenic challenge.

### 5.10 Effective Dose

The amount of the anti-CD20 antibody-drug conjugate which will be effective to treat a cancer or immune disorder of the invention can be determined by standard clinical techniques. In addition, in vitro assays may optionally be employed to help identify optimal dosage ranges. The precise dose to be employed in the formulation will also depend on the route of administration and the severity of the disease, and should be decided according to the judgment of the practitioner and each patient's circumstances. Effective doses may be extrapolated from dose-response curves derived from animal model test systems.

Toxicity and therapeutic efficacy of a particular anti-CD20 antibody-drug conjugate can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of particular anti-CD20 conjugates of the invention lies preferably within a range of circulating concentrations that include the ED₅₀ with little or no toxicity. The dosage may vary within this range depending upon the dosage form employed and the route of administration utilized. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Generally, the dosage of an anti-CD20 antibody-drug conjugate administered to treat a CD20-associated disorder is typically 0.1 mg/kg to 100 mg/kg of the patient's body weight, although subtherapeutic dosages may be administered when combination therapy is employed. Preferably, the dosage administered to a patient is between 0.1 mg/kg and 20 mg/kg of the patient's body weight, more preferably 1 mg/kg to 10 mg/kg of the patient's body weight. In other embodiments, the dosage of the anti-CD20 antibody-drug conjugate is 50 mg/m² to 1000 mg/m², more preferably 100 mg/m² to 750 mg/m², more preferably 200 mg/m² to 500 mg/m², and yet more preferably 300 mg/m² to 400 mg/m² of a patient's body surface area.

### 5.11 Kits

The invention also provides a pharmaceutical pack or kit comprising one or more containers filled with a nucleic acid or protein of the invention and optionally one or more pharmaceutical carriers. Optionally associated with such container(s) can be a notice in the form prescribed by a governmental agency regulating the manufacture, use or sale of pharmaceuticals or biological products, which notice reflects approval by the agency of manufacture, use or sale for human administration.

In one embodiment, a kit comprises an anti-CD20 antibody-drug conjugate of the invention. In other embodiments, a kit of the invention comprises components (e.g., antibody, linker and/or drug) for manufacturing a conjugate of the invention. A kit of the invention may optionally further comprise a pharmaceutical carrier.

### 5.12 Combination Therapy for Treatment of CD20-Expressing Cancers

The anti-CD20 ADCs of the invention can be administered together with treatment with irradiation or one or more chemotherapeutic agents. Such combinatorial administration can have an additive or synergistic effect on disease parameters. The combination therapy methods of the present invention provide the advantage of being able to administer reduced doses of irradiation or chemotherapeutic agents, including doses that may be subtherapeutic by themselves, which lowers the toxic and immunosuppressive side-effects of these therapies.

For irradiation treatment, the irradiation can be gamma rays or X-rays. For a general overview of radiation therapy, see Hellman, Chapter 12: Principles of Radiation Therapy Cancer, in: Principles and Practice of Oncology, DeVita et al., eds., 2nd. Ed., J.B. Lippencott Company, Philadelphia.

Useful classes of drugs include, but are not limited to, the following non-mutually exclusive classes of agents: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, and vinca alkaloids. Individual chemotherapeutics encompassed by the invention include but are not limited to an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16 and VM-26.

In a specific embodiment, an anti-CD20 ADC of the invention is administered concurrently with radiation therapy or one or more chemotherapeutic agents. In another specific embodiment, chemotherapy or radiation therapy is administered prior or subsequent to administration of a nucleic acid or protein of the invention, by at least an hour and up to several months, for example at least an hour, five hours, 12 hours, a day, a week, a month, or three months, prior or subsequent to administration of a nucleic acid or protein of the invention.

In a specific embodiment in which an anti-CD20 ADC of the invention is further conjugated to a pro-drug converting enzyme, the ADC is administered with a pro-drug. Administration of the pro-drug can be concurrent with administration of the ADC of the invention, or, more preferably, follows the administration of the ADC of the invention by at least an hour to up to one week, for example about five hours, 12 hours, or a day. Depending on the pro-drug converting enzyme administered, the pro-drug can be a benzoic acid mustard, an aniline mustard, a phenol mustard, p-hydroxyaniline mustard-glucuronide, epirubicin-glucuronide, adriamycin-N phenoxyaceryl, N-(4′-hydroxyphenyl acetyl)-palytoxin doxorubicin, melphalan, nitrogen mustard-cephalosporin, β-phenylenediamine, vinblastine derivative-cephalosporin, cephalosporin mustard, cyanophenylmethyl-β-D-gluco-pyranosiduronic acid, 5-(adaridin-1-yl-)2,4-dinitrobenzamide, or methotrexate-alanine.

Additionally, combination therapy may include administration of an agent that targets a receptor or receptor complex other than CD20 on the surface the cancerous cells. An example of such an agent is a second, non-CD20 antibody that binds to a molecule at the surface a cancerous cell. The antibody can be a polyclonal antibody, a monoclonal antibody, an epitope-binding antibody fragment, or another type of antibody derivative equivalent to those anti-CD20 derivatives described in Sections 5.1 and 5.3. In certain specific embodiments, the antibody is a multivalent antibody or a heteroconjugate. In such embodiments, the anti-CD20 ADC of the invention includes such a multivalent antibody or heteroconjugate. Another example is a ligand that targets such a receptor or receptor complex.

Preferably, such an antibody or ligand binds to a cell surface receptor on cancerous cells and enhances the cytotoxic effect of the anti-CD20 ADC, e.g., by delivering a cytostatic or cytotoxic signal to the cancer cell. Such agents need not be growth inhibitory or apoptotic on their own, but, in combination with anti-CD20 ADC, an enhanced effect on cytotoxicity beyond that induced by the anti-CD20 ADC alone can be achieved. In certain specific embodiments, the enhanced effect is approximately a 5%, 10% 15%, 20%, 25%, 30%, 40%, 50%, 75%, 100% or greater enhancement in the cytostatic or cytotoxic activity of a given amount or concentration of an anti-CD20 ADC of the invention. In one embodiment, the enhanced effect refers to an approximately 5%, 10% 15%, 20%, 25%, 30%, 40%, 50%, 75% reduction in the ED₅₀ of the CD20-ADC, i.e., the amount of the CD20-ADC capable of achieving the same cytotoxic or cytostatic effect is less than what would be required to achieve the same cytotoxic or cytostatic effect in the absence of administration of such agents that bind to receptor or receptor complexes other than CD20.

In certain embodiments, the method further comprises administering to the subject a cytotoxic or cytostatic agent. The cytotoxic or cytostatic agent is selected from the group consisting of an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, a vinca alkaloid, a purine antagonist, and a dihydrofolate reductase inhibitor. More specifically, the chemotherapeutic agent can be androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16, VM-26, azothioprine, mycophenolate mofetil, methotrexate, acyclovir, gangcyclovir, zidovudine, vidarabine, ribavarin, azidothymidine, cytidine arabinoside, amantadine, dideoxyuridine, iododeoxyuridine, poscarnet, or trifluridine.

In certain embodiments, the method further comprises administering to the subject a second antibody that binds to an antigen of the CD20-expressing cancer, and wherein the second antibody is not an anti-CD20 antibody. In specific embodiments, the second antibody is an anti-CD19 antibody, an anti-CD22 antibody, an anti-CD30 antibody, or an anti-CD40 antibody. In more specific embodiments, the second antibody is conjugated to a drug.

### 5.13 Combination Therapy for Treatment of Immune Disorders

The anti-CD20 ADCs of the invention can be administered together with one or more cytostatic, cytotoxic and/or immunosuppressive agents for the treatment and prevention of immune disorders. Additionally, combination therapy may include administration of an agent that targets a receptor or receptor complex other than CD20 on the surface immune cells. An example of such an agent is a second, non-CD20 antibody that binds to a molecule at the surface of an activated lymphocyte (e.g., an anti-CD30 antibody). Another example is a ligand that targets such a receptor or receptor complex. Preferably, such an antibody or ligand binds to a cell surface receptor on activated lymphocytes and enhances the cytotoxic or cytostatic effect of the anti-CD20 antibody by delivering a cytostatic or cytotoxic signal to the activated lymphocytes.

Such combinatorial administration can have an additive or synergistic effect on disease parameters.

With respect to therapeutic regimens, in a specific embodiment, an anti-CD20 ADC of the invention is administered concurrently with an immunsuppressive agent or a molecule that targets a lymphocyte cell surface receptor or receptor complex. In another specific embodiment, the immunosuppressive agent or lymphocyte cell surface receptor targeting-agent is administered prior or subsequent to administration of an ADC of the invention, by at least an hour and up to several months, for example at least an hour, five hours, 12 hours, a day, a week, a month, or three months, prior or subsequent to administration of an anti-CD20 ADC of the invention.

In certain embodiments, the method further comprises administering to the subject a chemotherapeutic agent. The chemotherapeutic agent is selected from the group consisting of an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, a vinca alkaloid, a purine antagonist, and a dihydrofolate reductase inhibitor. More specifically, the chemotherapeutic agent can be androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16, VM-26, azothioprine, mycophenolate mofetil, methotrexate, acyclovir, gangcyclovir, zidovudine, vidarabine, ribavarin, azidothymidine, cytidine arabinoside, amantadine, dideoxyuridine, iododeoxyuridine, poscarnet, or trifluridine.

In certain embodiments, the method further comprises administering to the subject a second antibody that binds to an antigen of the CD20-expressing cells, and wherein the second antibody is not an anti-CD20 antibody. In more specific embodiments, the second antibody is selected from the group consisting of an anti-CD19 antibody, an anti-CD22 antibody, an anti-CD30 antibody, and an anti-CD40 antibody. In certain embodiments, the second antibody is conjugated to a drug.

### 5.13.1 Immunosuppressive, Cytotoxic and Cytostatic Agents

A useful class of immunosuppressive, cytotoxic or cytostatic agents for practicing the combinatorial therapeutic regimens of the present invention include, but are not limited to, the following non-mutually exclusive classes of agents: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, and vinca alkaloids.

Individual immunosuppressive, cytotoxic or cytostatic agents encompassed by the invention include but are not limited to an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16 and VM-26.

In a preferred embodiment, the immunosuppressive, cytotoxic or cytostatic agent is an antimetabolite. The antimetabolite can be a purine antagonist (e.g. azothioprine) or mycophenolate mofetil), a dihydrofolate reductase inhibitor (e.g., methotrexate), acyclovir, gangcyclovir, zidovudine, vidarabine, ribavarin, azidothymidine, cytidine arabinoside, amantadine, dideoxyuridine, iododeoxyuridine, poscarnet, and trifluridine.

In another preferred embodiment, the immunosuppressive, cytotoxic or cytostatic agent is tacrolimus, cyclosporine or rapamycin.

In another preferred embodiment, the immunosuppressive agent is a glucocorticoid or glucocorticoid analogue. Examples of glucocorticoids useful in the present methods include cortisol and aldosterone. Examples of glucocorticoid analogues useful in the present methods include prednisone and dexamethasone.

In yet another preferred embodiment, the immunosuppressive agent is an anti-inflammatory agent, such as consisting arylcarboxylic derivatives, pyrazole-containing derivatives, oxicam derivatives and nicotinic acid derivatives. Classes of anti-inflammatory agents useful in the methods of the present invention include cyclooxygenase inhibitors, 5-lipoxygenase inhibitors, and leukotriene receptor antagonists.

Suitable cyclooxygenase inhibitors include meclofenamic acid, mefenamic acid, carprofen, diclofenac, diflunisal, fenbufen, fenoprofen, ibuprofen, indomethacin, ketoprofen, nabumetone, naproxen, sulindac, tenoxicam, tolmetin, and acetylsalicylic acid.

Suitable lipoxygenase inhibitors include redox inhibitors (e.g., catechol butane derivatives, nordihydroguaiaretic acid (NDGA), masoprocol, phenidone, Ianopalen, indazolinones, naphazatrom, benzofuranol, alkylhydroxylamine), and non-redox inhibitors (e.g., hydroxythiazoles, methoxyalkylthiazoles, benzopyrans and derivatives thereof, methoxytetrahydropyran, boswellic acids and acetylated derivatives of boswellic acids, and quinolinemethoxyphenylacetic acids substituted with cycloalkyl radicals), and precursors of redox inhibitors.

Other suitable lipoxygenase inhibitors include antioxidants (e.g., phenols, propyl gallate, flavonoids and/or naturally occurring substrates containing flavonoids, hydroxylated derivatives of the flavones, flavonol, dihydroquercetin, luteolin, galangin, orobol, derivatives of chalcone, 4,2′,4′-trihydroxychalcone, ortho-aminophenols, N-hydroxyureas, benzofuranols, ebselen and species that increase the activity of the reducing selenoenzymes), iron chelating agents (e.g., hydroxamic acids and derivatives thereof, N-hydroxyureas, 2-benzyl-1-naphthol, catechols, hydroxylamines, carnosol trolox C, catechol, naphthol, sulfasalazine, zyleuton, 5-hydroxyanthranilic acid and 4-(omega-arylalkyl)phenylalkanoic acids), imidazole-containing compounds (e.g., ketoconazole and itraconazole), phenothiazines, and benzopyran derivatives.

Yet other suitable lipoxygenase inhibitors include inhibitor eicosanoids (e.g., octadecatetraenoic, eicosatetraenoic, docosapentaenoic, eicosahexaenoic and docosahexaenoic acids and esters thereof, PGE1 (prostaglandin E1), PGA2 (prostaglandin A2), viprostol, 15-monohydroxyeicosatetraenoic, 15-monohydroxy-eicosatrienoic and 15-monohydroxyeicosapentaenoic acids, and leukotrienes B5, C5 and D5), compounds interfering with calcium flows, phenothiazines, diphenylbutylamines, verapamil, fuscoside, curcumin, chlorogenic acid, caffeic acid, 5,8,11,14-eicosatetrayenoic acid (ETYA), hydroxyphenylretinamide, Ionapalen, esculin, diethylcarbamazine, phenantroline, baicalein, proxicromil, thioethers, diallyl sulfide and di-(1-propenyl) sulfide.

Leukotriene receptor antagonists include calcitriol, ontazolast, Bayer Bay-x-1005, Ciba-Geigy CGS-25019C, ebselen, Leo Denmark ETH-615, Lilly LY-293111, Ono ONO-4057, Terumo TMK-688, Boehringer Ingleheim BI-RM-270, Lilly LY 213024, Lilly LY 264086, Lilly LY 292728, Ono ONO LB457, Pfizer 105696, Perdue Frederick PF 10042, Rhone-Poulenc Rorer RP 66153, SmithKline Beecham SB-201146, SmithKline Beecham SB-201993, SmithKline Beecham SB-209247, Searle SC-53228, Sumitamo SM 15178, American Home Products WAY 121006, Bayer Bay-o-8276, Warner-Lambert CI-987, Warner-Lambert CI-987BPC-15LY 223982, Lilly LY 233569, Lilly LY-255283, MacroNex MNX-160, Merck and Co. MK-591, Merck and CO. MK-886, Ono ONO-LB-448, Purdue Frederick PF-5901, Rhone-Poulenc Rorer RG 14893, Rhone-Poulenc Rorer RP 66364, Rhone-Poulenc Rorer RP 69698, Shionoogi S-2474, Searle SC-41930, Searle SC-50505, Searle SC-51146, Searle SC-52798, SmithKline Beecham SK&F-104493, Leo Denmark SR-2566, Tanabe T-757 and Teijin TEI-1338.

### 5.13.2 Lymphocyte Receptor Targeting Agents

Agents that are particularly useful in the present combinatorial methods are molecules that bind to lymphocyte cell surface, preferably against a receptor or receptor complex distinct from CD20. Besides CD20, a wide variety of receptors or receptor complexes expressed on lymphocyte surface are involved in regulating the proliferation, differentiation, and functions of different lymphocyte subsets. Such molecules can be targeted, for example, to provide additional cytostatic or cytotoxic signals to activated lymphocytes.

In one embodiment, suitable receptors for targeting alongside CD20 are immunoglobulin gene superfamily members, including but not limited to CD2, CD3, CD4, CD8, CD19, CD22, CD28, CD30, CD79, CD90, CD152/CTLA-4, PD-1, and ICOS (Barclay et al., 1997, The Leucocyte Antigen FactsBook, 2nd ed, Academic Press; Coyle and Gurtierrez-Ramos, 2001, Nature Immunol. 2:203-209). In another embodiment, TNF receptor superfamily members can be targeted, including but not limited to CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, and APO-3. (Locksley et al., 2001, Cell, 104, 487-501). In yet another embodiment, an integrin can be targeted, including but not limited to CD11a, CD11b, CD11c, CD18, CD29, CD41, CD49a, CD49b, CD49c, CD49d, CD49e, CD49f, CD103, and CD104 (Barclay et al., 1997, The Leucocyte Antigen FactsBook, 2nd ed, Academic Press). In yet other embodiments, a suitable receptor for targeting in addition to CD20 is a cytokine receptor (Fitzgerald et al., 2001, The Cytokine Factsbook, 2nd ed, Academic Press), a chemokine receptor (Luther and Cyster, 2001, Nature Immunol. 2:102-107; Gerard and Rollins, 2001, Nature Immunol. 2: 108-115), a major histocompatibility protein, a lectin (C-type, S-type, or I-type), or a complement control protein.

In one embodiment, agents that bind to these non-CD20 receptors or receptor complexes enhance the cytotoxic or cytostatic effect of the anti-CD20 ADC of the invention by delivering a cytostatic or cytotoxic substance to the activated lymphocytes. In certain embodiments, the cytostatic or cytotoxic substance that is delivered by an agent that binds a non-CD20 receptor is different from the drug that is delivered via an anti-CD20 antibody. In certain other embodiments, the cytostatic or cytotoxic substance that is delivered by an agent that binds a non-CD20 receptor is the same as the drug that is delivered via an anti-CD20 antibody. In combination with the anti-CD20 ADCs of the invention, an additive or synergistic effect on growth inhibition or apoptosis can be achieved in the targeted lymphocyte.

In another embodiment, agents against these receptors or receptor complexes need not be growth inhibitory or apoptotic on their own, but, in combination with a anti-CD20 ADC, an enhanced effect on growth inhibition or apoptosis beyond that induced by the anti-CD20 ADC alone can be achieved. In certain specific embodiments, the enhanced effect is approximately a 5%, 10% 15%, 20%, 25%, 30%, 40%, 50%, 75%, 100% or greater enhancement in the cytostatic or cytotoxic activity of a given amount or concentration of an anti-CD20 ADC of the invention. In one embodiment, the enhanced effect refers to an approximately 5%, 10% 15%, 20%, 25%, 30%, 40%, 50%, 75% reduction in the ED₅₀ of the CD20-ADC, i.e., the amount of the CD20-ADC capable of achieving the same cytotoxic or cytostatic effect is less than what would be required to achieve the same cytotoxic or cytostatic effect in the absence of administration of such agents that bind to receptor or receptor complexes other than CD20.

In one embodiment, targeting a non-CD20 receptor or receptor complex according to the methods of the present invention can be achieved by administering a ligand.

In another embodiment, targeting can be achieved by administering an antibody against the receptor or receptor complex. The antibody can be a polyclonal antibody, a monoclonal antibody, an epitope-binding antibody fragment, or another type of antibody derivative equivalent to those anti-CD20 derivatives described in Sections 5.1 and 5.3. In certain specific embodiments, the antibody is a multivalent antibody or a heteroconjugate. In such embodiments, the anti-CD20 ADC of the invention includes such a multivalent antibody or heteroconjugate, as described in Sections 5.1 and 5.3.

A number of antibodies suitable for co-administration with the anti-CD20 ADCs are known in the art, as will be recognized by the skilled artisan. Listed below are exemplary, non-limiting examples of such antibodies: the anti-CD2 antibodies include BTI-322 (Medimmune) and UMCD2; the anti-CD3 antibodies OKT3, “SMART” Anti-CD3 (Nuvion™; Protein Design Laboratories), FN18, UCHT1, 145-2C11, and HIT3a; the anti-CD5 antibodies H1211 (6T-003), HISM2 (6T-004), MEM-128 (6T-014), 7.8 (6T-080, OKT1, UCHT2, and BL1 a; the anti-CTLA-4 antibodies I 1D4, 10A8, 7F8, 4F10, ANC152.2/8H5, and BNI3.1; and the anti-PD-I antibody J43.

Natural ligands have also been defined for many of the receptors or receptor complexes (Barclay et al., 1997, The Leucocyte Antigen FactsBook, 2nd ed, Academic Press; Coyle and Gurtierrez-Ramos, 2001, Nature Immunol. 2:203-20; Locksley et al., 2001, Cell 104:487-501). Listed below are exemplary, non-limiting examples of such ligands: LFA-3, a ligand for CD2; CD80 and CD86, ligands for CD28 and CTLA-4; PD-L1 and PD-L2, ligands for PD-1; B7RP-1, a ligand for ICOS; CD70, a ligand for CD27; CD154, a ligand for CD40; FasL, a ligand for CD95/Fas; TNFa, a ligand for TNF-R1 and TNF-R2; TRANCE, a ligand for RANK, APRIL, a ligand for TACI; BLYS, a ligand for BCMA, TRAIL, a ligand for TRAIL-R1, -R2, -R3, and R4; and TWEAK, a ligand for APO-3.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

The invention is further described in the following examples which are in no way intended to limit the scope of the invention.

### 6. EXAMPLE: ANTI-CANCER ACTIVITY OF HIGH-POTENCY ANTI-CD20 ANTIBODY-DRUG CONJUGATES

### 6.1 Introduction

While anti-CD20 mAbs did not make effective therapeutic agents coupled to doxorubicin, it was unknown whether the use of significantly more potent drugs could result in active anti-CD20-based ADCs. To test this possibility we constructed conjugates using two anti-CD20-ADCs that incorporate high-potency cytotoxic agents such as Auristatin E that are >50 times more potent than doxorubicin. Two anti-CD20 mAbs, Rituxan and 1F5 were conjugated to monomethyl Auristatin E (MMAE) through a cathepsin B-cleavable valine-citrulline peptide linkage. We report here the surprising result that anti-CD20 ADCs using the highly potent cytotoxic drug MMAE can effectively and specifically kill CD20 positive tumor cells.

The anti-CD20 monoclonal antibody (mAb) Rituximab (Rituxan® has proven to be efficacious in the treatment of numerous B cell malignancies. Despite the success of Rituxan, a significant number of CD20-positive neoplasia remain refractive to this treatment or relapse after initial response. The efficacy of anti-CD20 therapy has been increased by labeling with radioisotope or by mAb co-administration with standard chemotherapy. CD20 does not efficiently internalize upon mAb ligation, thus targeting options have been limited to radionuclide payloads that can induce toxicity from the cell surface. Initial attempts to prepare active antibody-drag conjugates (ADCs) composed of anti-CD20 mAbs and drugs such as doxorubicin failed to demonstrate antitumor efficacy. We now demonstrate that anti-CD20 mAbs can be used to prepare ADCs that have potent antitumor activity when conjugated to MMAE, a derivative of the highly potent anti-mitotic agent Auristatin E. The conjugates, Rituxan-vcMMAE and 1F5-vcMMAE were found to be potent and selective, producing IC₅₀ values of 50 ng/ml following brief exposure. No toxicity was seen against CD20-negative cells treated at 2-3 logs higher of anti-CD20-drug conjugate or on CD20-positive cells treated with MMAE conjugated to an irrelevant mAb. ADCs prepared with anti-CD20 mAbs linked to doxorubicin were ineffective, suggesting the high potency of MMAE, which is greater than 50 times more potent than doxorubicin was key to this activity.

### 6.2 Materials and Methods

Cells and reagents. Rituxan (chimeric anti-CD20) was obtained from RX USA (Jamaica, N.Y.). Murine hybridoma line 1F5 producing IgG₂ₐ was previously reported (Press et al., Blood 1987; 69:584-591). The hybridoma was grown in RPMI-1640 media (Life Technologies Inc., Gaithersburg, Md.) supplemented with 10% fetal bovine serum. Antibody was purified from culture supernatants by protein A chromatography. Ramos, Raji and Daudi B cell lines were obtained from ATCC (Manassas, Va.) and the anaplastic large cell lymphoma (ALCL) line, Karpas 299 was obtained from the DSMZ (Braunschweig, Germany). All cell lines used were verified to be mycoplasma free by PCR. Goat-anti-mouse-FITC and goat-anti-human-FITC were from Jackson Immunoresearch, (West Grove, Pa.). Mouse anti-human IgG and rat anti-mouse IgG for conjugation purposes were prepared from hybridomas (mouse anti-human 1410 KG7, rat anti-mouse 187.1) obtained from ATCC.

Drug Synthesis. The synthesis of the activated val-cit linker used in both the auristatin and doxorubicin syntheses was modified from the previously described procedure (Firestone R A, Dubowehik G M, U.S. Pat. No. 6,214,345). The benzyl alcohol (0.37 g, 0.65 mmol) in DMF (6 mL) was treated with bis(4-nitrophenyl)carbonate (0.40 g, 2.0 eq.) followed by diisopropylethylamine (0.17 mL, 1.5 eq.). By HPLC analysis, the reaction was complete in 1 h. Solvent removal in vacuo gave a dark yellow oil that was suspended in EtOAc (10 mL) and triturated for 10 min. Ether (20 mL) was added and the ppt. further triturated for 5 min. The solid was collected and dried under high vacuum.

Yield: 0.26 g (56%).

The synthesis of auristatin E has been previously described (Pettit G R, and Barkoczy, J., 1997, U.S. Pat. No. 5,635,483, Pettit, G R, The Dolastatins, Prog. Chem. Org. Nat. Prod., 70, 1-79, 199). The monomethyl derivative of Auristatin E (MMAE) was prepared by replacing a protected form of monomethylvaline for N,N-dimethylvaline in the synthesis of auristatin B (Senter et al., US patent application). MMAE was then further modified with a linker that allows for conjugation to mAbs. Specifically, MMAE was modified with activated derivatives of maleimidocaproyl-valine-citrulline or maleimidocaproylphenylalanine-lysine that contained a p-aminobenzylcarbamate spacer between the MMAE and the linker.

The activated linker (60 mg, 84 μmol, 1.1 eq.), MMAE (56 mg, 76 μmol, 1.0 eq.), and HOBt (10 mg, 1.0 eq.) were dissolved in anhydrous DMF (2 mL) and pyridine (0.5 mL). The contents were stirred while being monitored by HPLC. The linker was not detected after 16 h. The reaction mixture was directly injected onto a reverse phase preparative HPLC column (Synergi MAX-RP, C₁₂ column 21.2 mm×25 cm, 10μ, 80 Å, using a gradient run of MeCN and 0.1% TFA at 25 mL/min from 10% to 100% over 40 min followed by 100% MeCN for 20 min). The fractions were immediately analyzed, pooled and concentrated to a sticky pale yellow solid. Addition of methylene chloride and hexanes (1:1) followed by evaporation led to vcMMAE as an off-white powder. Yield: 72 mg (70%). R_{f}0.36 (9:1 CH₂Cl₂—MeOH); ES-MS m/z 1316.7 [M+H]⁺; 1334.4 [M+NH₄]⁺; Uvλₘₐₓ 215, 248 nm.

The synthesis of doxorubicin with the val-cit linker described above has been previously described (U.S. Pat. No. 6,214,345, Dubowchik and Firestone, 1998). The activated linker (35 mg, 46 μmol, 1.1 eq.) and doxorubicun (30 mg, 1.1 eq.) were suspended in anhydrous DMF (3 mL). Diisopropylethylamine (4.2 μL, 1.1 eq.) was added and the reaction mixture became a homogeneous solution which was complete after 24 h. The reaction mixture was concentrated to 1.5 mL and loaded onto a SiO₂ column that was pre-wetted with methylene chloride. An eluant gradient of 100% CH₂Cl₂to 4:1 CH₂Cl₂—MeOH was used. Pooling of the desired fractions resulted in a red/orange solid product. Yield: 45 mg (83%). R_{f} 0.10 (9:1 CH₂Cl₂—MeOH); ES-MS m/z 1142.0 [M+H]⁺; UV λₘₐₓ 238, 254, 285, 495 nm.

Conjugate Preparation. Antibody Reduction. To 4.8 mL Rituxan (10 mg/mL) was added 600 uL of 500 mM sodium borate/500 mM NaCl, pH 8.0, followed by 600 uL of 100 mM DTT in water. After incubation at 37° C. for 30 min, the buffer was exchanged by elution over G25 resin equilibrated and eluted with PBS containing 1 mM DTPA (Aldrich). The thiol/Ab value was checked by determining the reduced antibody concentration from the solution's 280 nm absorbance, and the thiol concentration by reaction with DTNB (Aldrich) and determination of the absorbance at 412 nm.

Conjugation of the Reduced Antibody. The reduced mAb was split into two equal portions and chilled on ice. The drug-linker (vcMMAE or vcDox) was used as a frozen DMSO solution of known concentration, and the quantity of drug-linker added to the reaction mixture was calculated as follows:

L stock solution=V×[Ab]×Fold Excess/[Drug-Linker], where V and [Ab] are the volume and molar concentration of the reduced antibody solution, respectively. 2.3 mL cold PBS/DTPA was added to each of the two reduced antibody solutions. For the vcMMAE conjugate, 133.6 uL of 7.5 mM maleimidocaproyl-vcMAE stock solution was diluted into 1.47 mL acetonitrile. For the vcDox conjugate, 125.2 μL of 8.0 mM maleimidocaproyl-vcDox was diluted into 1.48 mL acetonitrile. The acetonitrile drug-linker solutions were chilled on ice, then added to the reduced antibody solutions. The reactions were terminated after 1 hr by the addition of a 20 fold molar excess of cysteine over maleimide. The reaction mixtures were concentrated by centrifugal ultrafiltration and purified by elution through de-salting G25 in PBS. ADCs were then filtered through 0.2 micron filters under sterile conditions and immediately frozen at −80C. ADCs were analyzed for 1) concentration, by UV absorbance; 2) aggregation, by size exclusion chromatography; 3) drug/Ab, by measuring unreacted thiols with DTNB, and 4) residual free drug, by reverse phase HPLC.

FACS analysis. To evaluate CD20 expression on cell lines, 1×10⁶ cells were combined with saturating levels (10 μg/ml) of mAb 1F5 in ice-cold PBS (staining media) for 30 min on ice and washed twice with ice-cold staining media to remove unbound mAb. Cells were then stained with secondary goat-anti-mouse-FITC, again at saturating levels (10 μg/ml) in ice-cold staining media, incubated for 30 minutes on ice and washed as described above. Labeled cells were examined by flow cytometry on a Becton Dickinson FACScan flow cytometer and were gated to exclude the non-viable cells. Data were analyzed using Winlist 4.0 software (Verity Software House) and the background-corrected mean fluorescence intensity was determined for each cell type. Quantitative determination of CD20 on cell surface was determined using a DAKO QUIFIKIT flow cytometric indirect immunofluorescence assay as described by the manufacturer (DAKO A/S, Glostrup, Denmark).

Flow cytometry for evaluation of binding. To evaluate binding of ADC to cells, 4×10⁶ Raji cells were combined with serial dilutions of mAb IFS, Rituxan or their ADCs in ice cold staining media for 30 minutes on ice and washed twice with ice cold staining medium. Cells were then incubated with goat anti-mouse-IgG FITC (for 1F5 and 1F5 ADC) or goat anti-human-IgG-FITC (for Rituxan or Rituxan-ADC) at 10 μg/ml on ice for 30 minutes and washed as described above. Labeled cells were then run on the flow cytometer and analyzed as above.

Cytotoxicity assays. Cytotoxicity was measured by Alamar Blue (Biosource International) dye reduction assay according to manufacturers directions (Nakayama et al. Journal Of Immunological Methods 1997; 204:205-208). Briefly, a 40% solution (w/v) of Alamar Blue was freshly prepared in complete media just prior to adding to cultures. The Alamar Blue solution was added at cell densities of 105 cells/ml for the Ramos, Raji, and Karpas cell lines, and at a cell density of 4×10⁵ cells/ml for the Daudi cell line. After 92 h following drug exposure. Alamar Blue solution was added to cells to constitute 10% culture volume. The cells were incubated for 4 h and dye reduction measured on a Fusion HT fluorescent plate reader (Packard Instruments, Meriden, Conn.).

Microscopy for mAb/ ADC trafficking. Ramos cells were incubated with 5 ug/ml Rituximab, Rituximab-vcMMAE or Rituximab-vcDox in complete media at 37 [symbol] C for 1, 4 and 24 h. At the designated times cells were harvested by centrifugation, fixed and permeabilized by paraformaldehyde/saponin (Cytofix/Cytoperm™ Buffer, BD PharMingen, San Diego, Calif.). After washing with the Perm/Wash™ buffer (BD PharMingen) and blocking with goat IgG, cells were stained with a goat anti-human IgG Fc_specific FITC conjugate (Jackson ImmunoResearch Laboratories, Inc., West Grove, Pa.). The localization of fluorescence signals was then visualized using a Deltavision confocal fluorescence microscope equipped with digital analysis software.

Xenograft models of human CD20-positive disease. For the localized non Hodgkin's lymphoma model, 2×10⁶ Ramos NHL cells were implanted into the right flank of SCID mice. Therapy with Rituximab, Rituxan-vcMMAE, or Rituxan-vcDox was initiated when the tumor size in each group of 5 animals averaged ˜100 mm³. Treatment consisted of intraperitoneal injections of mAb or ADC every 4 days for 3 injections. Tumor size was determined using the formula (L×W²)/2.

### 6.3 Results

Preparation of Antibody-Drug Conjugates. Auristatins are highly potent antimitotic agents related in structure to the marine natural product, dolastatin 10. These agents act by inhibiting the polymerization of tubulin in dividing cells (Pettit G R, and Barkoczy, J., 1997, U.S. Pat. No. 5,635,483, Pettit, G R, The Dolastatins, Prog. Chem. Org. Nat. Prod., 70, 1-79, 1997). The monomethyl derivative of auristatin E (MMAE) was prepared by replacing a protected form of monomethylvaline for valine in the synthesis of auristatin E (Senter et al. US patent application). MMAE was then further modified with maleimidocaproyl-valine-citrulline to result in vcMMAE that contained a p-aminobenzylcarbamate spacer between the MMAE and the linker. Similarly doxorubicin was modified with maleimidocaproyl-valine-citrulline to result in vcDox that contained a p-aminobenzylcarbamate spacer between doxorubicin and the linker. The resulting drug derivatives used in these studies are shown in FIG. 1.

The linkers used for drug attachment were designed to release active drug in the presence of intracellular proteolytic enzymes, such as cathepsin B (Dubowchik, G M, Firestone, R A, Bioorg. Med. Chem. Lett., 8, 1998. 3341-3346). Upon proteolytic cleavage, the linker between the peptide and the drug undergoes a fragmentation reaction, leading to the release of MMAE. Cathepsin B leads to the rapid release of drug from both the phe-lys and val-cit derivatives. ADCs prepared with this enzyme-cleavable linker system are highly stable in the absence of active enzyme. Characteristics of the Rituxan-ADCs used in these studies are shown in Table 1 below:

TABLE 1
Characteristics of the Rituxan ADCs used in this study

| | Rituxan-vcMMAE | Rituxan-vcDox |
|---|---|---|
| Concentration | 3.2 mg/mL | 3.5 mg/mL |
| Drug/Ab | 7 | 5.6 |
| Residual free drug | <0.5% | <0.5% |

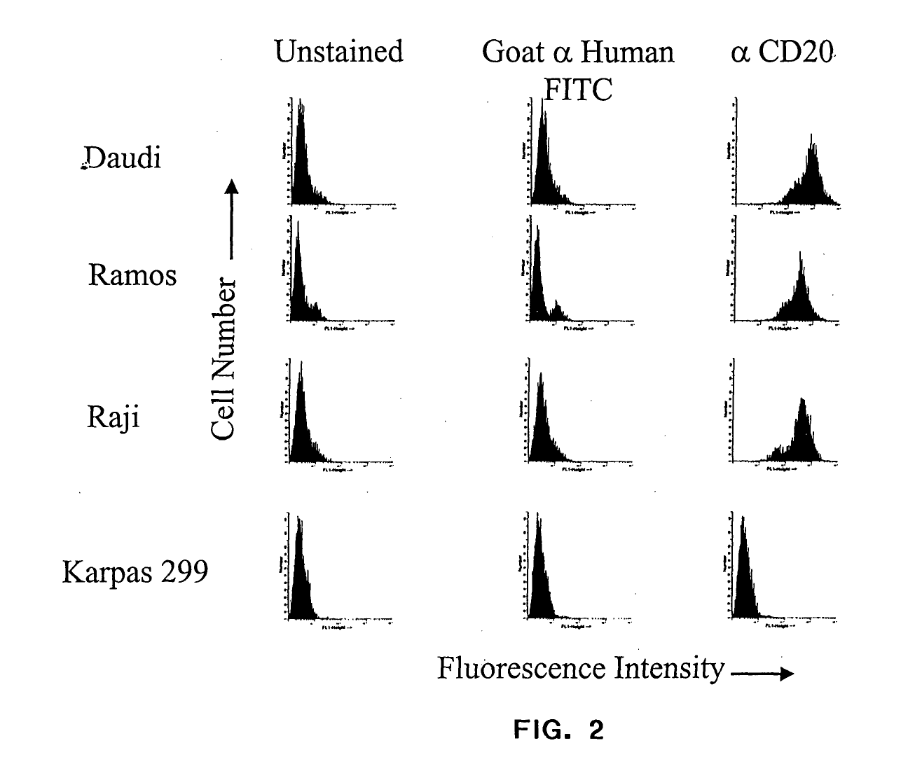

Cell characterization and sensitivity to unconjugated drugs. The human B cell lymphoma lines Daudi, Raji and Ramos, and the large cell lymphoma line, Karpas 299, were evaluated by flow cytometry to assess their relative CD20 expression levels using Rituxan followed by a goat anti-human IgG-FITC reagent. The resultant fluorescent intensity profiles for each population are shown in FIG. 2. Daudi, Ramos and Raji cells were all positive for CD20 at decreasing levels of intensity. The ALCL line Karpas showed no CD20 -staining and was used as an antigen negative control cell in these studies. Quantitative estimation of CD20 density on cell surface of these cells was determined using a QUIFIKIT indirect immunofluorescence flow cytometric (Poncelet and Carayon, J. Immunol. Methods 1985 85:65-74). This assay correlates mAb reactive cell fluorescence with the number of bound primary antibody molecules on the cells by relating cell fluorescence to that emanating from the surface of a similarly stained series of beads coated with well defined quantities of mAb molecules. Using this method the density of CD20 were determined to be 3.71×10⁵, 4.07×10⁵, 4.52×10⁵ and 0 copies per cell for on Ramos, Raji, Daudi and Karpas cell lines respectively.

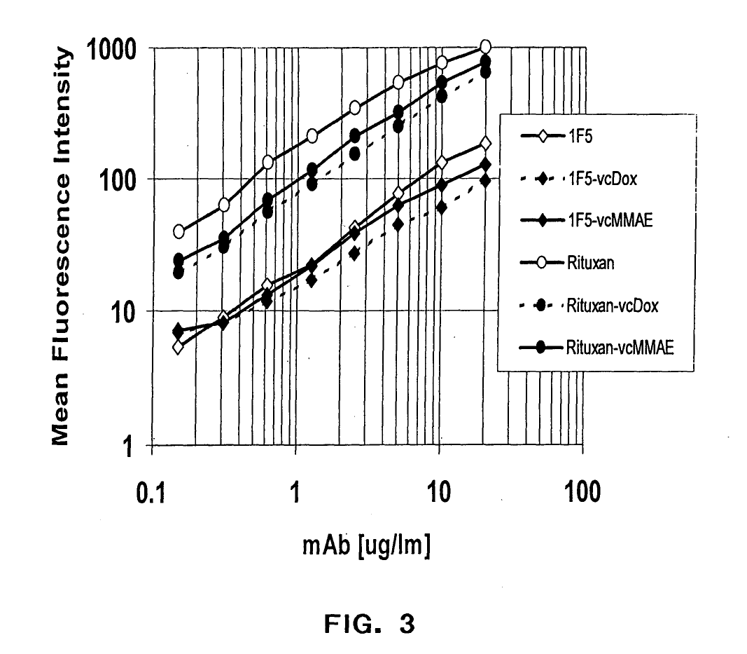

Binding of mAbs and ADCs to CD20-positive cells. To evaluate the cell binding characteristics of Rituxan, 1F5 and their ADCs, aliquots of Daudi cells were combined with increasing concentrations of either mAb alone or their respective ADCs, incubated on ice to block antigen modulation and washed. Goat anti-human IgG-FITC then detected the bound mAb or ADC for Rituxan and irrelevant IgG and goat anti-mouse IgG-FITC for 1F5 as described in Materials and Methods. FIG. 3 shows fluorescence intensity versus concentration for cells stained with the mAb alone or mAb conjugated to doxorubicin or MMAE. The signal due to Rituxan was ˜0.8 log greater than that of 1F5 (MFI=800 U and 150 units respectively at 10 ug/ml), suggesting an increased binding of CD20 by Rituxan over 1F5. Under these conditions neither mAb was saturating at levels as high as 20 ug/ml, suggesting CD20 is present at high density on Raji cells. Conjugation of Rituxan or 1F5 with either doxorubicin or MMAE had minimal effect on the binding when compared to the parental, uncojugated mAbs. Binding of doxorubicin-containing ADCs being somewhat more attenuated than those with MMAB (FIG. 3).

Cell sensitivity to unconjugated drugs. The cells were initially evaluated for relative sensitivity to doxorubicin and MMAE. Cells were exposed to drugs for 2 h, washed, replated in fresh media and incubated for an additional 92 h. Four h. prior to harvest, cells were incubated with Alamar Blue, a reducible dye that provides readout of cell viability (Nakayarna et al., 1997). Table 2 below shows the sensitivities (IC₅₀) of Daudi, Ramos, Raji and Karpas 299 cells to MMAE and doxorubicin. MMAE was found to be 57-200 times more potent than doxorubicin against these cell lines:

TABLE 2
Sensitivity of Cells to Unconjugated Drugs

| | MMAE | Doxorubicin IC50 | Activity Ratio |
|---|---|---|---|
| Cell Type | IC50 [nM] | [nM] | MMAE/Doxorubicin |
| Daudi (CD20+) | 0.65 | 140 | 215.4 |
| Ramos (CD20+) | 1 | 225 | 225.0 |
| Raji (CD20+) | 3.5 | 200 | 57.1 |
| Karpas (CD20−) | 3 | 650 | 216.7 |

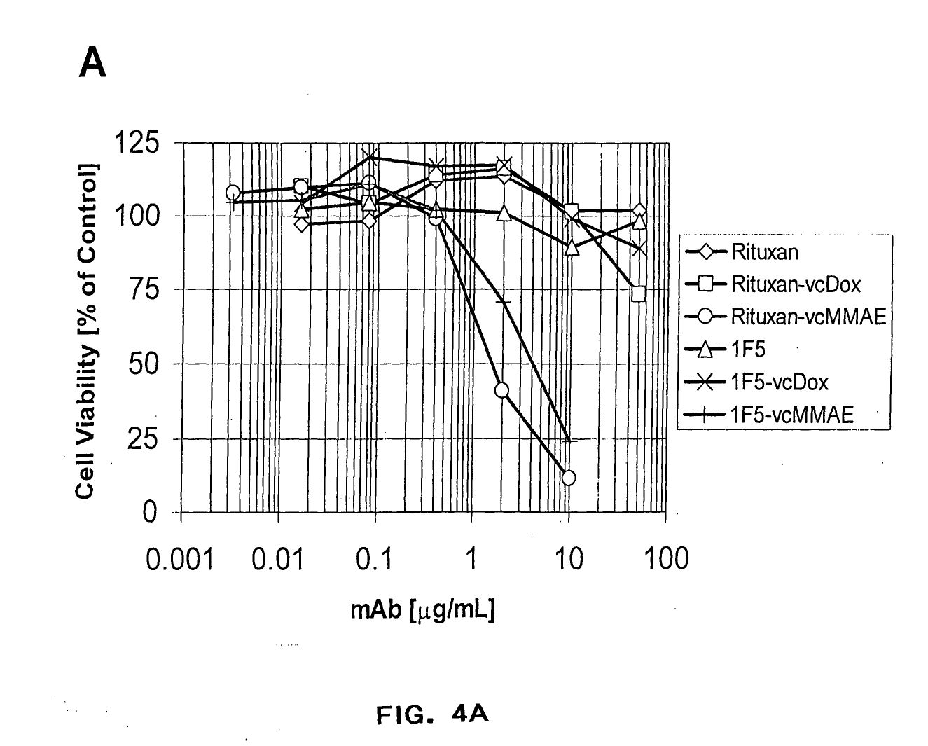

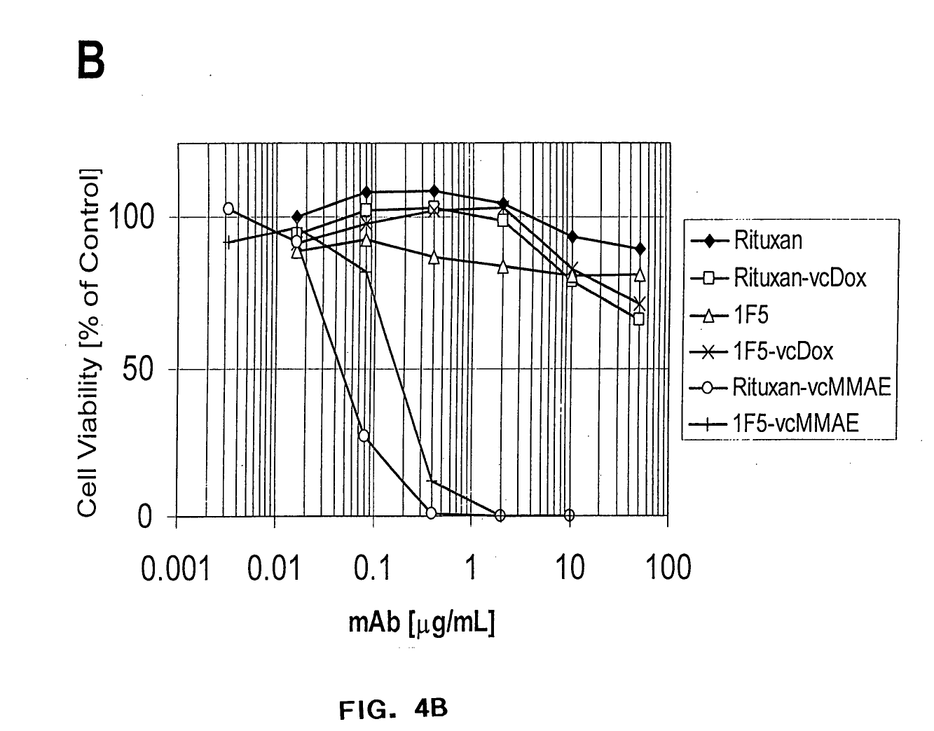

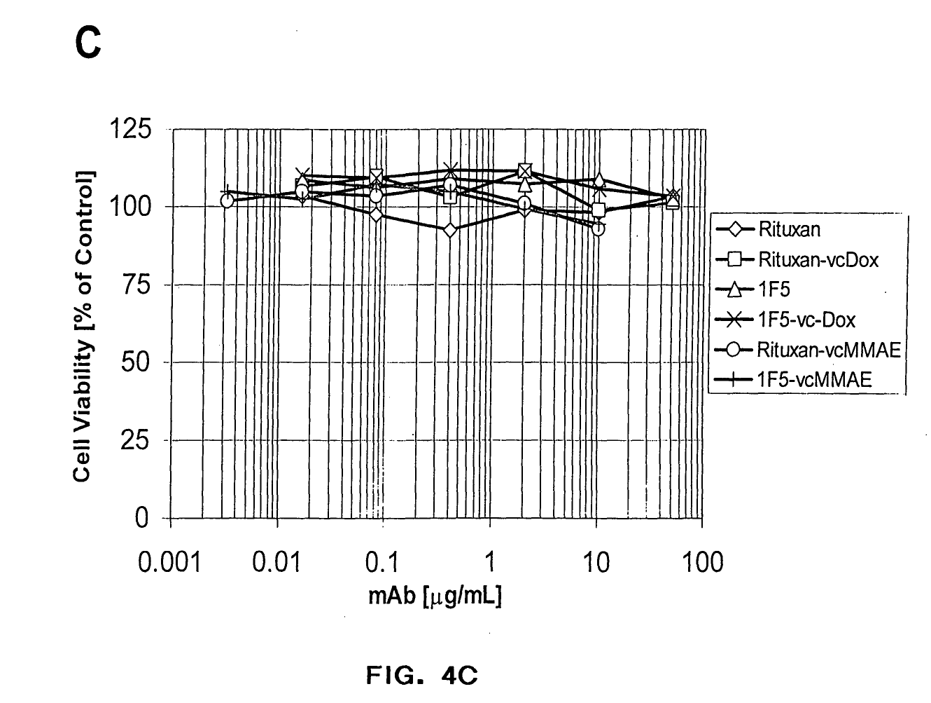

Sensitivity of cells to ADCs. To evaluate the potency and selectivity of various ADCs, cells were incubated for 2 h to allow binding, washed to remove unbound ADC, replated in fresh media and returned to incubation for an additional 92 h. As described above, 4 h prior to harvest, cells were incubated with Alamar Blue and cell viability assessed. Rituxan and 1F5 conjugated to MMAE produced IC₅₀ of 1.4 and 4 ug/ml respectively on Raji cells (FIG. 4A). In contrast, Rituxan and 1F5 conjugated to doxorubicin did not affect significant toxicity on these cells at the highest levels tested (50 ug/ml). MMAE conjugated to an irrelevant IgG, cAC10, was also not cytotoxic to these cells at up to 50 ug/ml. FIG. 4B shows a similar study performed on the Ramos cells. Rituxan and 1F5 conjugated to MMAE were highly cytotoxic to Ramos cells with resultant IC₅₀ₛ of 45 ng/ml and 180 ng/ml respectively on Ramos cells. Again, neither Rituxan nor 1F5 conjugated to doxorubicin nor an irrelevant IgG conjugated to MMAE could affect an IC₅₀ on these cells at the highest levels tested (50 ug/ml). Similar results were obtained with the CD20 positive line Daudi. IC₅₀ values for each ADC and the relative expression of CD20 on these cells is summarized in Table 3:

TABLE 3
Sensitivity of Cells to ADC

| | Rituxan- | Rituxan- | 1F5- | ¹IgG- | CD20 |
|---|---|---|---|---|---|
| | cvMMAE | vcDox | vcMMAE | vcMMAE | Density |
| Cell Type | ²IC₅₀ [μg/ml] | IC₅₀ [μg/ml] | IC₅₀ [μg/ml] | IC₅₀ [μg/ml] | Copy/Cell × 10³ |
| Daudi | 2 | >50 | 1.5 | >50 | 4.5 |
| Ramos | 0.045 | >50 | 0.18 | >50 | 3.7 |
| Raji | 1.5 | >50 | 4.0 | >50 | 4.1 |
| Karpas | >50 | >50 | >50 | >50 | 0 |

To further demonstrate the selectivity of these ADCs, the CD20-negative cell line Karpas 299 was treated with Rituxan and 1F5 conjugated to MMAE, as well as the associated controls. None of the ADCs were cytotoxic to CD20-negative Karpas cells up to the maximum level tested (50 ug/ml; FIG. 4C).

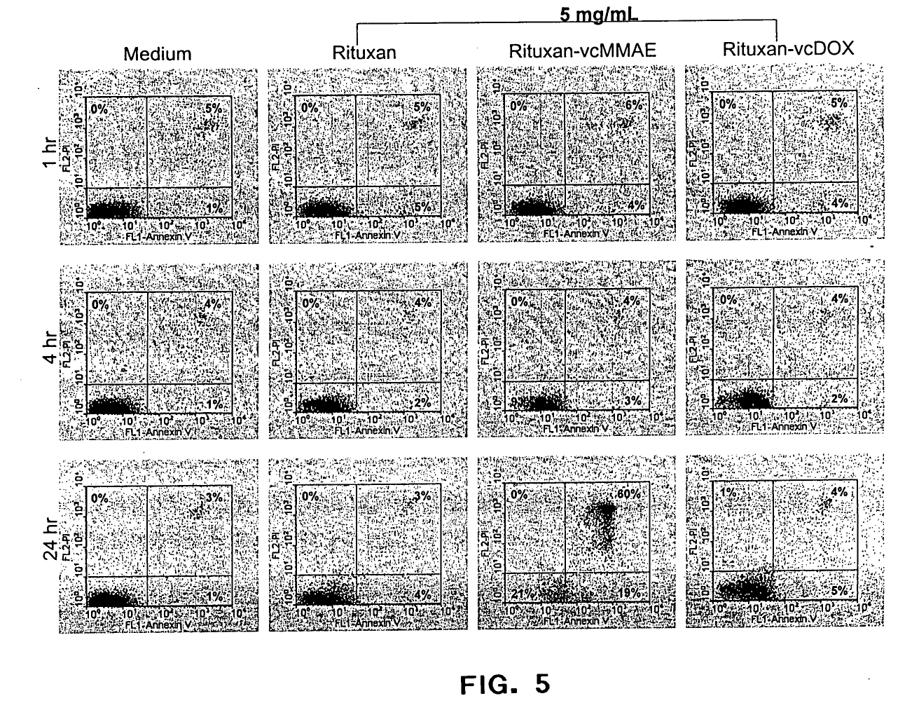

Induction of Apoptosis. These data suggested that Anti-CD20-vcMMAE caused cytotoxicity not seen with the unmodified mAb or those modified with vcDox. To examine the ability of Rituximab-vcMMAE to induce apoptosis, Ramos cells were incubated with equivalent levels (5 μg/ml) of either Rituximab, Rituximab-vcMMAE, or Rituximab-vcDOX, or in medium alone. At designated time points cells were removed from cultures and the degrees of apoptosis and cell death were determined by Annexin V binding to the cell surface and loss of propidium iodide (PI) inclusion respectively (FIG. 5). Annexin V binds phosphatidylserine that is translocated from the inner plasma membrane to the cell surface at the onset of apoptosis (Martin et al., 1995, J. Exp. Med. 182:1545-1556). Staining with PI, normally excluded from viable cells, indicates loss of membrane integrity in dead or dying cells (Vermes et al., 1995, J. Immunol. Meth. 184:39-51). Incubation of Ramos cells with either Rituximab or Rituximab-vcDOX up to 24 h did not induce apoptosis or cell death significantly over that seen with the medium control. For Rituximab or Rituximab-vcDOX treated cells the range of apoptotic cells (determined as a ratio of Annexin V^{positive}/PI^{negative}) was 2% -5% while that of dead cells (Annexin V^{positive}/PI^{positive}) was 3% -5%, compared to 1% of apoptotic cells and 3% -5% of dead cells in the medium control. The percentages of apoptotic and dead cells in the culture treated with Rituximab-vcMMAE were comparable to other cultures up to 4 h post-exposure, however, these increased to 19% and 60%, respectively, after 24 hr of incubation (FIG. 5). These data indicate that at equivalent mAb concentrations, Rituximab-vcMMAE selectively induced apoptosis and cell death not seen with Rituximab or Rituximab-vcDOX.

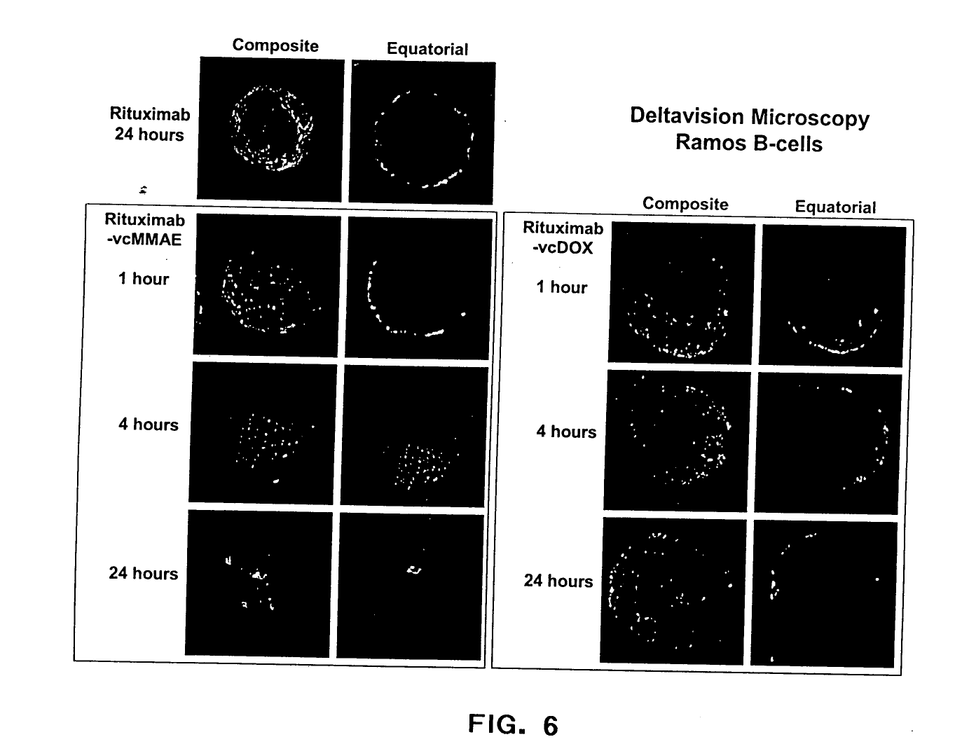

Internalization of the mAb-drug conjugate. Cytotoxicity data suggested that anti-CD20-vcMMAE conjugates were able to effectively deliver MMAE to the cell interior. To address the possibility that this ADC may traffic differently than the vcDox conjugate or the mAb alone, the cellular localization of Rituximab, Rituximab-vcMMAE and Rituximab-vcDox was followed subsequent to binding to CD20 on the cell surface, using confocal indirect fluorescence microscopy. Ramos cells were incubated with Rituximab or ADCs for 1, 4 or 24 h, fixed, permeabilized and stained with a goat anti-human IgG Fc_specific FITC as described in Materials and Methods. The localization of fluorescence signals was then determined (FIG. 6). Some patching and capping of Rituximab-CD20 complexes could be detected as early as 30 min after incubation of cells with Rituximab (data not shown). A pile-up composite photomicrograph shows that these patched and capped complexes remained detectable amidst diffuse surface staining up to 24 hr post-incubation and confocal examination through the cell equatorial section indicated only localization to the cell membrane and no fluorescence signal localized to the inside of cells, suggesting minimal internalization of the Rituximab-CD20 complexes. Rituximab-vcMMAE and Rituximab-vcDOX also produced patching and capping of the ADC-CD20 complexes. These signals were initially more focused than those of the Rituximab-CD20 complexes. Both Rituximab-vcMMAE-CD20 and Rituximab-vcDOX-CD20 complexes showed extensive, punctate staining after 4 or 24 h yet as shown in a equatorial section image, the Rituximab-vcDOX-CD20 complexes remained primarily on the surface. Remarkably, microscopy through midsection of cells treated with Rituximab-vcMMAE clearly revealed ADC localized to the cell interior within 4 h of treatment (FIG. 6). Neither binding nor internalization of the mAb or ADC could be detected on CD20-negative Karpas cells (data not shown). Internalization of Rituximab-ADC-CD20 complexes was also accompanied by a concomitant decrease in Rituximab ADC-CD20 complexes on cell surface as detected by flow cytometry on intact cells (data not shown).

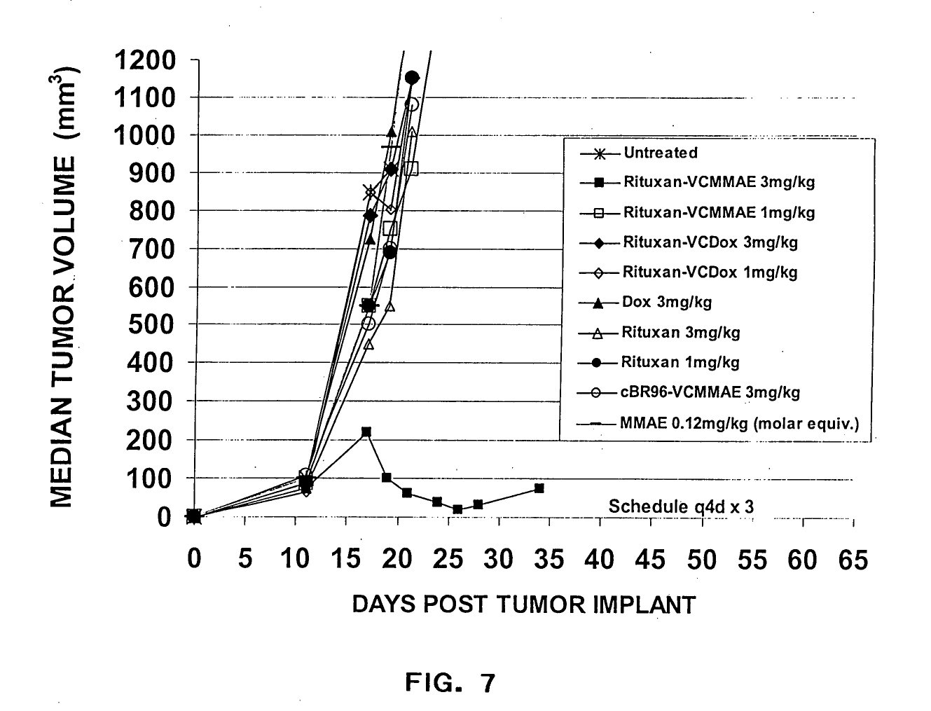

In vivo efficacy of Rituximab-vcMMAE against NHL xenografts. To examine the therapeutic potential of an anti-CD20-MMAE conjugate, the in vivo activity of Rituximab-vcMMAE was evaluated in SCID mice using Ramos NHL cells. Others have demonstrated that anti-CD20 mAbs alone are efficacious in disseminated models of NHL in mice (Hooijberg et al., 1995, Cancer Res. 55:840-846) and for purposes of evaluating the ADC we have established a more stringent model of localized disease where mAb alone was not effective. SCID mice were implanted with 5×10⁶ Ramos cells into the flank and the tumor size in each group of 5 animals was allowed to progress to an average size of 100 mm³ before therapy with mAb or ADCs was initiated. Treatment consisted of intraperitoneal injections of mAb or ADC every 4 days for 3 injections using 1 and 3 mg/kg/injection. Tumors in the untreated and animals treated with Rituximab or Rituximab-vcDox grew rapidly and reached an average of >800 mm³ by day 20. Only Rituximab-vcMMAE at 3 mg/kg produced a significant delay in tumor growth at all concentrations tested in a dose dependent manner (FIG. 7).

### 7. SPECIFIC EMBODIMENTS, CITATION OF REFERENCES

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various references, including patent applications, patents, and scientific publications, are cited herein, the disclosures of which are incorporated herein by reference in their entireties.

1. An anti-CD20 antibody-cytotoxic agent conjugate, wherein the cytotoxic agent of the anti-CD20 antibody-cytotoxic agent conjugate has an IC₅₀ of between 40-fold and 4,000-fold less than the IC₅₀ of doxorubicin, with the proviso that the cytotoxic agent is not a radioisotope or a toxin, and wherein the IC₅₀ of each of the cytotoxic agent and doxorubicin is measured by a method comprising:
(a) culturing one or more CD20-expressing cell populations in the presence of one or more concentrations of the cytotoxic agent for a 72-to 96-hour period;
(b) culturing one or more CD20-expressing cell populations in the presence of one or more concentrations of doxorubicin for a 72- to 96-hour period; and
(c) identifying a concentration of the cytotoxic agent and doxorubicin, respectively, at which 50% fewer cells in the CD20-expressing cell populations of steps (a) and (b), respectively, are viable at the end of the period relative to a CD20-expressing cell population cultured in the absence of the cytotoxic agent and doxorubicin,
wherein the CD20-expressing cell populations of steps (a), (b) and (c) are of the same cell type and are cultured under the same conditions,
and wherein the concentration of the cytotoxic agent and doxorubicin identified in step (c) is the IC₅₀ of the cytotoxic agent and doxorubicin, respectively.

2-5. (canceled)

6. An anti-CD20 antibody-cytotoxic agent conjugate, wherein the anti-CD20 antibody-cytotoxic agent conjugate has an IC₅₀ of between 40-fold and 4,000-fold less than the IC₅₀ of an anti-CD20 antibody-doxorubicin conjugate, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, and wherein the IC₅₀ of each of the anti-CD20 antibody-cytotoxic agent conjugate and the anti-CD20 antibody-doxorubicin conjugate is measured by a method comprising:
(a) culturing one or more CD20-expressing cell populations in the presence of one or more concentrations of the anti-CD20 antibody-cytotoxic agent conjugate for a 72- to 96-hour period;
(b) culturing one or more CD20-expressing cell populations in the presence of one or more concentrations of the anti-CD20 antibody-doxorubicin conjugate for a 72- to 96-hour period; and
(c) identifying a concentration of the anti-CD20 antibody-cytotoxic agent conjugate and the anti-CD20 antibody-doxorubicin conjugate, respectively, at which 50% fewer cells in the CD20-expressing cell populations of steps (a) and (b), respectively, are viable at the end of the period relative to a CD20-expressing cell population type cultured in the absence of the anti-CD20 antibody-cytotoxic agent conjugate and the anti-CD20 antibody-doxorubicin conjugate,
wherein the CD20-expressing cell populations of steps (a), (b) and (c) are of the same cell type and are cultured under the same conditions,
and wherein the concentration of the anti-CD20 antibody-cytotoxic agent conjugate and the anti-CD20 antibody-doxorubicin conjugate identified in step (c) is the IC₅₀ of the anti-CD20 antibody-cytotoxic agent conjugate and the anti-CD20 antibody-doxorubicin conjugate, respectively.

7-11. (canceled)

12. An anti-CD20 antibody-cytotoxic agent conjugate, wherein the conjugate has a rate of accumulation in a CD20-expressing cell that is between 20-fold and 5,000-fold greater than the rate of accumulation of an unconjugated form of the anti-CD20 antibody in the CD20-expressing cell, wherein the rates of accumulation of the conjugate and of the unconjugated form of the antibody are measured by a method comprising:
(a) culturing a population of the CD20-expressing cell with the conjugate;
(b) culturing a population of the CD20-expressing cell with the unconjugated antibody, wherein the populations of steps (a) and (b) are cultured under the same conditions; and
(c) measuring the amount of the conjugate and unconjugated antibody accumulated in the populations of steps (a) and (b), respectively.

13-17. (canceled)

18. An anti-CD20 antibody-cytotoxic agent conjugate, wherein the conjugate has a rate of accumulation in a CD20-expressing cell that is between 20-fold and 5,000-fold greater than the rate of accumulation of an anti-CD20 antibody-doxorubicin conjugate in a CD20-expressing cell of the same cell type, wherein the anti-CD20 antibody-cytotoxic agent conjugate and the anti-CD20 antibody-doxorubicin conjugate comprise the same anti-CD20 antibody, and wherein the rates of accumulation of the anti-CD20 antibody-cytotoxic agent conjugate and of the anti-CD20 antibody-doxorubicin conjugate are measured by a method comprising:
(a) culturing a population of the CD20-expressing cell with the anti-CD20 antibody-cytotoxic agent conjugate;
(b) culturing a population of the CD20-expressing cell with the anti-CD20 antibody-doxorubicin conjugate, wherein the populations of steps (a) and (b) are cultured under the same conditions; and
(c) measuring the amount of the anti-CD20 antibody-cytotoxic agent conjugate and anti-CD20 antibody-doxorubicin conjugate accumulated in the populations of steps (a) and (b), respectively.

19-21. (canceled)

22. An anti-CD20 antibody-cytotoxic agent conjugate, wherein the conjugate exhibits an accumulation in a non-peripheral region inside a CD20-expressing cell that is between 1.5-fold and 5,000-fold greater than the accumulation of an unconjugated form of the anti-CD20 antibody in the CD20-expressing cell, wherein the accumulation of the conjugate and of the unconjugated form of the antibody are measured by a method comprising:
(a) culturing a population of the CD20-expressing cell with the conjugate;
(b) culturing a population of the CD20-expressing cell with the unconjugated form of the anti-CD20 antibody; and
(c) detecting by confocal fluorescence microscopy localization of the conjugate and the unconjugated form of the anti-CD20 antibody in the populations of steps (a) and (b), respectively,
wherein the populations of steps (a) and (b) are cultured under the same conditions and for the same period of time, and wherein the conjugate exhibits an accumulation in the CD20-expressing cell that is between 1.5-fold and 5,000-fold greater than the accumulation of the unconjugated form of the anti-CD20 antibody in the CD20-expressing cell if:
(i) between 1.5-fold and 5,000-fold as many cells of the population of step (a) contain a detectable amount of the conjugate in a non-peripheral region as the number of cells of the population of step (b) contain the unconjugated form of the antibody in a non-peripheral region; or
(ii) the accumulation of the conjugate in a non-peripheral region of the majority of CD20-expressing cells of the population of step (a) is between 1.5-fold and 5,000-fold greater than the accumulation of the unconjugated form of the anti-CD20 antibody in the majority of CD20-expressing cells of the population of step (b).

23-25. (canceled)

26. An anti-CD20 antibody-cytotoxic agent conjugate, wherein the conjugate exhibits an accumulation in a non-peripheral region inside a CD20-expressing cell that is between 1.5-fold and 5,000-fold greater than the accumulation of an anti-CD20 antibody-doxorubicin conjugate, wherein the anti-CD20 antibody-cytotoxic agent conjugate and the anti-CD20 antibody-doxorubicin conjugate comprise the same anti-CD20 antibody, in the CD20-expressing cell, wherein the accumulation of the conjugate and of the anti-CD20 antibody-doxorubicin conjugate are measured by a method comprising:
(a) culturing a population of the CD20-expressing cell with the conjugate;
(b) culturing a population of the CD20-expressing cell with the anti-CD20 antibody-doxorubicin conjugate; and
(c) detecting by confocal fluorescence microscopy localization of the conjugate and the anti-CD20 antibody-doxorubicin conjugate in the populations of steps (a) and (b), respectively,
wherein the populations of steps (a) and (b) are cultured under the same conditions and for the same period of time, and wherein the conjugate exhibits an accumulation in the CD20-expressing cell that is between 1.5-fold and 5,000-fold greater than the accumulation of the anti-CD20 antibody-doxorubicin conjugate in the CD20-expressing cell if:
(i) between 1.5-fold and 5,000-fold as many cells of the population of step (a) contain a detectable amount of the conjugate in a non-peripheral region as the number of cells of the population of step (b) contain the anti-CD20 antibody-doxorubicin conjugate in a non-peripheral region; or
(ii) the accumulation of the conjugate in a non-peripheral region of the majority of CD20-expressing cells of the population of step (a) is between 1.5-fold and 5,000-fold greater than the accumulation of the anti-CD20 antibody-doxorubicin conjugate in the majority of CD20-expressing cells of the population of step (b).

27-64. (canceled)

65. A pharmaceutical composition comprising an anti-CD20 antibody-cytotoxic agent conjugate, wherein the cytotoxic agent of the anti-CD20 antibody-cytotoxic agent conjugate has an IC₅₀ of between 40-fold and 4,000-fold less than the IC₅₀ of doxorubicin, with the proviso that the cytotoxic agent is not a radioisotope or a toxin, and wherein the IC₅₀ of each of the cytotoxic agent and doxorubicin is measured by a method comprising:
(a) culturing one or more CD20-expressing cell populations in the presence of one or more concentrations of the cytotoxic agent for a 72-to 96-hour period;
(b) culturing one or more CD20-expressing cell populations in the presence of one or more concentrations of doxorubicin for a 72- to 96-hour period; and
(c) identifying a concentration of the cytotoxic agent and doxorubicin, respectively, at which 50% fewer cells in the CD20-expressing cell populations of steps (a) and (b), respectively, are viable at the end of the period relative to a CD20-expressing cell population cultured in the absence of the cytotoxic agent and doxorubicin,
wherein the CD20-expressing cell populations of steps (a), (b) and (c) are of the same cell type and are cultured under the same conditions,
and wherein the concentration of the cytotoxic agent and doxorubicin identified in step (c) is the IC₅₀ of the cytotoxic agent and doxorubicin, respectively.

66. A pharmaceutical composition comprising an anti-CD20 antibody-cytotoxic agent conjugate, wherein the anti-CD20 antibody-cytotoxic agent conjugate has an IC₅₀ of between 40-fold and 4,000-fold less than the IC₅₀ of an anti-CD20 antibody-doxorubicin conjugate, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, and wherein the IC₅₀ of each of the anti-CD20 antibody-cytotoxic agent conjugate and the anti-CD20 antibody-doxorubicin conjugate is measured by a method comprising:
(a) culturing one or more CD20-expressing cell populations in the presence of one or more concentrations of the anti-CD20 antibody-cytotoxic agent conjugate for a 72- to 96-hour period;
(b) culturing one or more CD20-expressing cell populations in the presence of one or more concentrations of the anti-CD20 antibody-doxorubicin conjugate for a 72- to 96-hour period; and
(c) identifying a concentration of the anti-CD20 antibody-cytotoxic agent conjugate and the anti-CD20 antibody-doxorubicin conjugate, respectively, at which 50% fewer cells in the CD20-expressing cell populations of steps (a) and (b), respectively, are viable at the end of the period relative to a CD20-expressing cell population cultured in the absence of the anti-CD20 antibody-cytotoxic agent conjugate and the anti-CD20 antibody-doxorubicin conjugate,
wherein the CD20-expressing cell populations of steps (a), (b) and (c) are of the same cell type and are cultured under the same conditions,
and wherein the concentration of the anti-CD20 antibody-cytotoxic agent conjugate and the anti-CD20 antibody-doxorubicin conjugate identified in step (c) is the IC₅₀ of the anti-CD20 antibody-cytotoxic agent conjugate and the anti-CD20 antibody-doxorubicin conjugate, respectively.

67. A pharmaceutical composition comprising an anti-CD20 antibody-cytotoxic agent conjugate, wherein the conjugate has a rate of accumulation in a CD20-expressing cell that is between 20-fold and 5,000-fold greater than the rate of accumulation of an unconjugated form of the anti-CD20 antibody in the CD20-expressing cell, with the proviso that the cytotoxic agent is not a radioisotope or a toxin, wherein the rates of accumulation of the conjugate and of the unconjugated form of the antibody are measured by a method comprising:
(a) culturing a population of the CD20-expressing cell with the conjugate;
(b) culturing a population of the CD20-expressing cell with the unconjugated antibody, wherein the populations of steps (a) and (b) are cultured under the same conditions; and
(c) measuring the amount of the conjugate and unconjugated antibody accumulated in the populations of steps (a) and (b), respectively.

68. A pharmaceutical composition comprising an anti-CD20 antibody-cytotoxic agent conjugate, wherein the conjugate has a rate of accumulation in a CD20-expressing cell that is between 20-fold and 5,000-fold greater than the rate of accumulation of an anti-CD20 antibody-doxorubicin conjugate in a CD20-expressing cell of the same cell type, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, and wherein the rates of accumulation of the anti-CD20 antibody-cytotoxic agent conjugate and of the anti-CD20 antibody-doxorubicin conjugate are measured by a method comprising:
(a) culturing a population of the CD20-expressing cell with the anti-CD20 antibody-cytotoxic agent conjugate;
(b) culturing a population of the CD20-expressing cell with the anti-CD20 antibody-doxorubicin conjugate, wherein the populations of steps (a) and (b) are cultured under the same conditions; and
(c) measuring the amount of the anti-CD20 antibody-cytotoxic agent conjugate and anti-CD20 antibody-doxorubicin conjugate accumulated in the populations of steps (a) and (b), respectively.

69. A pharmaceutical composition comprising an anti-CD20 antibody-cytotoxic agent conjugate, wherein the conjugate exhibits an accumulation in a non-peripheral region inside a CD20-expressing cell that is between 1.5-fold and 5,000-fold greater than the accumulation of an unconjugated form of the anti-CD20 antibody in the CD20-expressing cell, with the proviso that the cytotoxic agent is not a radioisotope or a toxin, wherein the accumulation of the conjugate and of the unconjugated form of the antibody are measured by a method comprising:
(a) culturing a population of the CD20-expressing cell with the conjugate;
(b) culturing a population of the CD20-expressing cell with the unconjugated form of the anti-CD20 antibody; and
(c) detecting by confocal fluorescence microscopy localization of the conjugate and the unconjugated form of the anti-CD20 antibody in the populations of steps (a) and (b), respectively,
wherein the populations of steps (a) and (b) are cultured under the same conditions and for the same period of time, and wherein the conjugate exhibits an accumulation in the CD20-expressing cell that is between 1.5-fold and 5,000-fold greater than the accumulation of the unconjugated form of the anti-CD20 antibody in the CD20-expressing cell if:
(i) between 1.5-fold and 5,000-fold as many cells of the population of step (a) contain a detectable amount of the conjugate in a non-peripheral region as the number of cells of the population of step (b) contain the unconjugated form of the antibody in a non-peripheral region; or
(ii) the accumulation of the conjugate in a non-peripheral region of the majority of CD20-expressing cells of the population of step (a) is between 1.5-fold and 5,000-fold greater than the accumulation of the unconjugated form of the anti-CD20 antibody in the majority of CD20-expressing cells of the population of step (b).

70. A pharmaceutical composition comprising an anti-CD20 antibody-cytotoxic agent conjugate, wherein the conjugate exhibits an accumulation in a non-peripheral region inside a CD20-expressing cell that is between 1.5-fold and 5,000-fold greater than the accumulation of an anti-CD20 antibody-doxorubicin conjugate, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, in the CD20-expressing cell, wherein the accumulation of the conjugate and of the anti-CD20 antibody-doxorubicin conjugate are measured by a method comprising:
(a) culturing a population of the CD20-expressing cell with the conjugate;
(b) culturing a population of the CD20-expressing cell with the anti-CD20 antibody-doxorubicin conjugate; and
(c) detecting by confocal fluorescence microscopy localization of the conjugate and the anti-CD20 antibody-doxorubicin conjugate in the populations of steps (a) and (b), respectively,
wherein the populations of steps (a) and (b) are cultured under the same conditions and for the same period of time, and wherein the conjugate exhibits an accumulation in the CD20-expressing cell that is between 1.5-fold and 5,000-fold greater than the accumulation of the anti-CD20 antibody-doxorubicin conjugate in the CD20-expressing cell if:
(i) between 1.5-fold and 5,000-fold as many cells of the population of step (a) contain a detectable amount of the conjugate in a non-peripheral region as the number of cells of the population of step (b) contain the anti-CD20 antibody-doxorubicin conjugate in a non-peripheral region; or
(ii) the accumulation of the conjugate in a non-peripheral region of the majority of CD20-expressing cells of the population of step (a) is between 1.5-fold and 5,000-fold greater than the accumulation of the anti-CD20 antibody-doxorubicin conjugate in the majority of CD20-expressing cells of the population of step (b).

71. A method of treating a CD20-expressing cancer, comprising administering to a subject in need of such treatment an effective amount of an anti-CD20 antibody-cytotoxic agent conjugate, wherein the cytotoxic agent of the anti-CD20 antibody-cytotoxic agent conjugate has an IC₅₀ of between 40-fold and 4,000-fold less than the IC₅₀ of doxorubicin, with the proviso that the cytotoxic agent is not a radioisotope or a toxin, and wherein the IC₅₀ of each of the cytotoxic agent and doxorubicin is measured by a method comprising:
(a) culturing one or more CD20-expressing cell populations in the presence of one or more concentrations of the cytotoxic agent for a 72-to 96-hour period;
(b) culturing one or more CD20-expressing cell populations in the presence of one or more concentrations of doxorubicin for a 72- to 96-hour period; and
(c) identifying a concentration of the cytotoxic agent and doxorubicin, respectively, at which 50% fewer cells in the CD20-expressing cell populations of steps (a) and (b), respectively, are viable at the end of the period relative to a CD20-expressing cell population cultured in the absence of the cytotoxic agent and doxorubicin,
wherein the CD20-expressing cell populations of steps (a), (b) and (c) are of the same cell type and are cultured under the same conditions,
and wherein the concentration of the cytotoxic agent and doxorubicin identified in step (c) is the IC₅₀ of the cytotoxic agent and doxorubicin, respectively.

72. A method of treating a CD20-expressing cancer, comprising administering to a subject in need of such treatment an effective amount of an anti-CD20 antibody-cytotoxic agent conjugate, wherein the anti-CD20 antibody-cytotoxic agent conjugate has an IC₅₀ of between 40-fold and 4,000-fold less than the IC₅₀ of an anti-CD20 antibody-doxorubicin conjugate, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, and wherein the IC₅₀ of each of the anti-CD20 antibody-cytotoxic agent conjugate and the anti-CD20 antibody-doxorubicin conjugate is measured by a method comprising:
(a) culturing one or more CD20-expressing cell populations in the presence of one or more concentrations of the anti-CD20 antibody-cytotoxic agent conjugate for a 72- to 96-hour period;
(b) culturing one or more CD20-expressing cell populations in the presence of one or more concentrations of the anti-CD20 antibody-doxorubicin conjugate for a 72- to 96-hour period; and
(c) identifying a concentration of the anti-CD20 antibody-cytotoxic agent conjugate and the anti-CD20 antibody-doxorubicin conjugate, respectively, at which 50% fewer cells in the CD20-expressing cell populations of steps (a) and (b), respectively, are viable at the end of the period relative to a CD20-expressing cell population cultured in the absence of the anti-CD20 antibody-cytotoxic agent conjugate and the anti-CD20 antibody-doxorubicin conjugate,
wherein the CD20-expressing cell populations of steps (a), (b) and (c) are of the same cell type and are cultured under the same conditions,
and wherein the concentration of the anti-CD20 antibody-cytotoxic agent conjugate and the anti-CD20 antibody-doxorubicin conjugate identified in step (c) is the IC₅₀ of the anti-CD20 antibody-cytotoxic agent conjugate and the anti-CD20 antibody-doxorubicin conjugate, respectively.

73. A method of treating a CD20-expressing cancer, comprising administering to a subject in need of such treatment an effective amount of an anti-CD20 antibody-cytotoxic agent conjugate, wherein the conjugate has a rate of accumulation in a CD20-expressing cell that is between 20-fold and 5,000-fold greater than the rate of accumulation of an unconjugated form of the anti-CD20 antibody in the CD20-expressing cell, with the proviso that the cytotoxic agent is not a radioisotope or a toxin, wherein the rates of accumulation of the conjugate and of the unconjugated form of the antibody are measured by a method comprising:
(a) culturing a population of the CD20-expressing cell with the conjugate;
(b) culturing a population of the CD20-expressing cell with the unconjugated antibody, wherein the populations of steps (a) and (b) are cultured under the same conditions; and
(c) measuring the amount of the conjugate and unconjugated antibody accumulated in the populations of steps (a) and (b), respectively.

74. A method of treating a CD20-expressing cancer, comprising administering to a subject in need of such treatment an effective amount of an anti-CD20 antibody-cytotoxic agent conjugate, wherein the conjugate has a rate of accumulation in a CD20-expressing cell that is between 20-fold and 5,000-fold greater than the rate of accumulation of an anti-CD20 antibody-doxorubicin conjugate in a CD20-expressing cell of the same cell type, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, and wherein the rates of accumulation of the anti-CD20 antibody-cytotoxic agent conjugate and of the anti-CD20 antibody-doxorubicin conjugate are measured by a method comprising:
(a) culturing a population of the CD20-expressing cell with the anti-CD20 antibody-cytotoxic agent conjugate;
(b) culturing a population of the CD20-expressing cell with the anti-CD20 antibody-doxorubicin conjugate, wherein the populations of steps (a) and (b) are cultured under the same conditions; and
(c) measuring the amount of the anti-CD20 antibody-cytotoxic agent conjugate and anti-CD20 antibody-doxorubicin conjugate accumulated in the populations of steps (a) and (b), respectively.

75. A method of treating a CD20-expressing cancer, comprising administering to a subject in need of such treatment an effective amount of an anti-CD20 antibody-cytotoxic agent conjugate, wherein the conjugate exhibits an accumulation in a non-peripheral region inside a CD20-expressing cell that is between 1.5-fold and 5,000-fold greater than the accumulation of an unconjugated form of the anti-CD20 antibody in the CD20-expressing cell, with the proviso that the cytotoxic agent is not a radioisotope or a toxin, wherein the accumulation of the conjugate and of the unconjugated form of the antibody are measured by a method comprising:
(a) culturing a population of the CD20-expressing cell with the conjugate;
(b) culturing a population of the CD20-expressing cell with the unconjugated form of the anti-CD20 antibody; and
(c) detecting by confocal fluorescence microscopy localization of the conjugate and the unconjugated form of the anti-CD20 antibody in the populations of steps (a) and (b), respectively,
wherein the populations of steps (a) and (b) are cultured under the same conditions and for the same period of time, and wherein the conjugate exhibits an accumulation in the CD20-expressing cell that is between 1.5-fold and 5,000-fold greater than the accumulation of the unconjugated form of the anti-CD20 antibody in the CD20-expressing cell if:
(i) between 1.5-fold and 5,000-fold as many cells of the population of step (a) contain a detectable amount of the conjugate in a non-peripheral region as the number of cells of the population of step (b) contain the unconjugated form of the antibody in a non-peripheral region; or
(ii) the accumulation of the conjugate in a non-peripheral region of the majority of CD20-expressing cells of the population of step (a) is between 1.5-fold and 5,000-fold greater than the accumulation of the unconjugated form of the anti-CD20 antibody in the majority of CD20-expressing cells of the population of step (b).

76. A method of treating a CD20-expressing cancer, comprising administering to a subject in need of such treatment an effective amount of an anti-CD20 antibody-cytotoxic agent conjugate, wherein the conjugate exhibits an accumulation in a non-peripheral region inside a CD20-expressing cell that is between 1.5-fold and 5,000-fold greater than the accumulation of an anti-CD20 antibody-doxorubicin conjugate, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, in the CD20-expressing cell, wherein the accumulation of the conjugate and of the anti-CD20 antibody-doxorubicin conjugate are measured by a method comprising:
(a) culturing a population of the CD20-expressing cell with the conjugate;
(b) culturing a population of the CD20-expressing cell with the anti-CD20 antibody-doxorubicin conjugate; and
(c) detecting by confocal fluorescence microscopy localization of the conjugate and the anti-CD20 antibody-doxorubicin conjugate in the populations of steps (a) and (b), respectively,
wherein the populations of steps (a) and (b) are cultured under the same conditions and for the same period of time, and wherein the conjugate exhibits an accumulation in the CD20-expressing cell that is between 1.5-fold and 5,000-fold greater than the accumulation of the anti-CD20 antibody-doxorubicin conjugate in the CD20-expressing cell if:
(i) between 1.5-fold and 5,000-fold as many cells of the population of step (a) contain a detectable amount of the conjugate in a non-peripheral region as the number of cells of the population of step (b) contain the anti-CD20 antibody-doxorubicin conjugate in a non-peripheral region; or
(ii) the accumulation of the conjugate in a non-peripheral region of the majority of CD20-expressing cells of the population of step (a) is between 1.5-fold and 5,000-fold greater than the accumulation of the anti-CD20 antibody-doxorubicin conjugate in the majority of CD20-expressing cells of the population of step (b).

77. (canceled)

78. A method of treating an immune disorder involving CD20-expressing cells, comprising administering to a subject in need of such treatment an effective amount of an anti-CD20 antibody-cytotoxic agent conjugate, wherein the cytotoxic agent of the anti-CD20 antibody-cytotoxic agent conjugate has an IC₅₀ of between 40-fold and 4,000-fold less than the IC₅₀ of doxorubicin, with the proviso that the cytotoxic agent is not a radioisotope or a toxin, and wherein the IC₅₀ of each of the cytotoxic agent and doxorubicin is measured by a method comprising:
(a) culturing one or more CD20-expressing cell populations in the presence of one or more concentrations of the cytotoxic agent for a 72-to 96-hour period;
(b) culturing one or more CD20-expressing cell populations in the presence of one or more concentrations of doxorubicin for a 72- to 96-hour period; and
(c) identifying a concentration of the cytotoxic agent and doxorubicin, respectively, at which 50% fewer cells in the CD20-expressing cell populations of steps (a) and (b), respectively, are viable at the end of the period relative to a CD20-expressing cell population cultured in the absence of the cytotoxic agent and doxorubicin,
wherein the CD20-expressing cell populations of steps (a), (b) and (c) are of the same cell type and are cultured under the same conditions,
and wherein the concentration of the cytotoxic agent and doxorubicin identified in step (c) is the IC₅₀ of the cytotoxic agent and doxorubicin, respectively.

79. A method of treating an immune disorder involving CD20-expressing cells, comprising administering to a subject in need of such treatment an effective amount of an anti-CD20 antibody-cytotoxic agent conjugate, wherein the anti-CD20 antibody-cytotoxic agent conjugate has an IC₅₀ of between 40-fold and 4,000-fold less than the IC₅₀ of an anti-CD20 antibody-doxorubicin conjugate, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, and wherein the IC₅₀ of each of the anti-CD20 antibody-cytotoxic agent conjugate and the anti-CD20 antibody-doxorubicin conjugate is measured by a method comprising:
(a) culturing one or more CD20-expressing cell populations in the presence of one or more concentrations of the anti-CD20 antibody-cytotoxic agent conjugate for a 72- to 96-hour period;
(b) culturing one or more CD20-expressing cell populations in the presence of one or more concentrations of the anti-CD20 antibody-doxorubicin conjugate for a 72- to 96-hour period; and
(c) identifying a concentration of the anti-CD20 antibody-cytotoxic agent conjugate and the anti-CD20 antibody-doxorubicin conjugate, respectively, at which 50% fewer cells in the CD20-expressing cell populations of steps (a) and (b), respectively, are viable at the end of the period relative to a CD20-expressing cell population cultured in the absence of the anti-CD20 antibody-cytotoxic agent conjugate and the anti-CD20 antibody-doxorubicin conjugate,
wherein the CD20-expressing cell populations of steps (a), (b) and (c) are of
the same cell type and are cultured under the same conditions,
and wherein the concentration of the anti-CD20 antibody-cytotoxic agent conjugate and the anti-CD20 antibody-doxorubicin conjugate identified in step (c) is the IC₅₀ of the anti-CD20 antibody-cytotoxic agent conjugate and the anti-CD20 antibody-doxorubicin conjugate, respectively.

80. A method of treating an immune disorder involving CD20-expressing cells, comprising administering to a subject in need of such treatment an effective amount of an anti-CD20 antibody-cytotoxic agent conjugate, wherein the conjugate has a rate of accumulation in a CD20-expressing cell that is between 20-fold and 5,000-fold greater than the rate of accumulation of an unconjugated form of the anti-CD20 antibody in the CD20-expressing cell, with the proviso that the cytotoxic agent is not a radioisotope or a toxin, and wherein the rates of accumulation of the conjugate and of the unconjugated form of the antibody are measured by a method comprising:
(a) culturing a population of the CD20-expressing cell with the conjugate;
(b) culturing a population of the CD20-expressing cell with the unconjugated antibody, wherein the populations of steps (a) and (b) are cultured under the same conditions; and
(c) measuring the amount of the conjugate and unconjugated antibody accumulated in the populations of steps (a) and (b), respectively.

81. A method of treating an immune disorder involving CD20-expressing cells, comprising administering to a subject in need of such treatment an effective amount of an anti-CD20 antibody-cytotoxic agent conjugate, wherein the conjugate has a rate of accumulation in a CD20-expressing cell that is between 20-fold and 5,000-fold greater than the rate of accumulation of an anti-CD20 antibody-doxorubicin conjugate in a CD20-expressing cell of the same cell type, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, and and wherein the rates of accumulation of the anti-CD20 antibody-cytotoxic agent conjugate and of the anti-CD20 antibody-doxorubicin conjugate are measured by a method comprising:
(a) culturing a population of the CD20-expressing cell with the anti-CD20 antibody-cytotoxic agent conjugate;
(b) culturing a population of the CD20-expressing cell with the anti-CD20 antibody-doxorubicin conjugate, wherein the populations of steps (a) and (b) are cultured under the same conditions; and
(c) measuring the amount of the anti-CD20 antibody-cytotoxic agent conjugate and anti-CD20 antibody-doxorubicin conjugate accumulated in the populations of steps (a) and (b), respectively.

82. A method of treating an immune disorder involving CD20-expressing cells, comprising administering to a subject in need of such treatment an effective amount of an anti-CD20 antibody-cytotoxic agent conjugate, wherein the conjugate exhibits an accumulation in a non-peripheral region inside a CD20-expressing cell that is between 1.5-fold and 5,000-fold greater than the accumulation of an unconjugated form of the anti-CD20 antibody in the CD20-expressing cell, with the proviso that the cytotoxic agent is not a radioisotope or a toxin, wherein the accumulation of the conjugate and of the unconjugated form of the antibody are measured by a method comprising:
(a) culturing a population of the CD20-expressing cell with the conjugate;
(b) culturing a population of the CD20-expressing cell with the unconjugated form of the anti-CD20 antibody; and
(c) detecting by confocal fluorescence microscopy localization of the conjugate and the unconjugated form of the anti-CD20 antibody in the populations of steps (a) and (b), respectively,
wherein the populations of steps (a) and (b) are cultured under the same conditions and for the same period of time, and wherein the conjugate exhibits an accumulation in the CD20-expressing cell that is between 1.5-fold and 5,000-fold greater than the accumulation of the unconjugated form of the anti-CD20 antibody in the CD20-expressing cell if:
(i) between 1.5-fold and 5,000-fold as many cells of the population of step (a) contain a detectable amount of the conjugate in a non-peripheral region as the number of cells of the population of step (b) contain the unconjugated form of the antibody in a non-peripheral region; or
(ii) the accumulation of the conjugate in a non-peripheral region of the majority of CD20-expressing cells of the population of step (a) is between 1.5-fold and 5,000-fold greater than the accumulation of the unconjugated form of the anti-CD20 antibody in the majority of CD20-expressing cells of the population of step (b).

83. A method of treating an immune disorder involving CD20-expressing cells, comprising administering to a subject in need of such treatment an effective amount of an anti-CD20 antibody-cytotoxic agent conjugate, wherein the conjugate exhibits an accumulation in a non-peripheral region inside a CD20-expressing cell that is between 1.5-fold and 5,000-fold greater than the accumulation of an anti-CD20 antibody-doxorubicin conjugate, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, in the CD20-expressing cell, wherein the accumulation of the conjugate and of the anti-CD20 antibody-doxorubicin conjugate are measured by a method comprising:
(a) culturing a population of the CD20-expressing cell with the conjugate;
(b) culturing a population of the CD20-expressing cell with the anti-CD20 antibody-doxorubicin conjugate; and
(c) detecting by confocal fluorescence microscopy localization of the conjugate and the anti-CD20 antibody-doxorubicin conjugate in the populations of steps (a) and (b), respectively,
wherein the populations of steps (a) and (b) are cultured under the same conditions and for the same period of time, and wherein the conjugate exhibits an accumulation in the CD20-expressing cell that is between 1.5-fold and 5,000-fold greater than the accumulation of the anti-CD20 antibody-doxorubicin conjugate in the CD20-expressing cell if:
(i) between 1.5-fold and 5,000-fold as many cells of the population of step (a) contain a detectable amount of the conjugate in a non-peripheral region as the number of cells of the population of step (b) contain the anti-CD20 antibody-doxorubicin conjugate in a non-peripheral region; or
(ii) the accumulation of the conjugate in a non-peripheral region of the majority of CD20-expressing cells of the population of step (a) is between 1.5-fold and 5,000-fold greater than the accumulation of the anti-CD20 antibody-doxorubicin conjugate in the majority of CD20-expressing cells of the population of step (b).

84-95. (canceled)

96. A kit comprising in a first container, an anti-CD20 antibody, and in a second container, a cytotoxic agent, wherein the cytotoxic agent has an IC₅₀ of between 40-fold and 4,000-fold less than the IC₅₀ of doxorubicin, with the proviso that the cytotoxic agent is not a radioisotope or a toxin, and wherein the IC₅₀ of each of the cytotoxic agent and doxorubicin is measured by a method comprising:
(a) culturing one or more CD20-expressing cell populations in the presence of one or more concentrations of the cytotoxic agent for a 72-to 96-hour period;
(b) culturing one or more CD20-expressing cell populations in the presence of one or more concentrations of doxorubicin for a 72- to 96-hour period; and
(c) identifying a concentration of the cytotoxic agent and doxorubicin, respectively, at which 50% fewer cells in the CD20-expressing cell populations, respectively, are viable at the end of the period relative to a CD20-expressing cell population cultured in the absence of the cytotoxic agent and doxorubicin,
wherein the CD20-expressing cell populations of steps (a), (b) and (c) are of the same cell type and are cultured under the same conditions,
and wherein the concentration of the cytotoxic agent and doxorubicin identified in step (c) is the IC₅₀ of the cytotoxic agent and doxorubicin, respectively.

97. (canceled)

98. A kit comprising in a first container, an anti-CD20 antibody, and in a second container, a cytotoxic agent, wherein upon conjugation of the anti-CD20 antibody and the drug, the resulting conjugate has a rate of accumulation in a CD20-expressing cell that is between 20-fold and 5,000-fold greater than the rate of accumulation of an unconjugated form of the anti-CD20 antibody in the CD20-expressing cell, with the proviso that the cytotoxic agent is not a radioisotope or a toxin, and wherein the rates of accumulation of the conjugate and of the unconjugated form of the antibody are measured by a method comprising:
(a) culturing a population of the CD20-expressing cell with the conjugate;
(b) culturing a population of the CD20-expressing cell with the unconjugated antibody, wherein the populations of steps (a) and (b) are cultured under the same conditions; and
(c) measuring the amount of the conjugate and unconjugated antibody accumulated in the populations of steps (a) and (b), respectively.

99. A kit comprising in a first container, an anti-CD20 antibody, and in a second container, a cytotoxic agent, wherein upon conjugation of the anti-CD20 antibody and the drug, the resulting conjugate has a rate of accumulation in a CD20-expressing cell that is between 20-fold and 5,000-fold greater than the rate of accumulation of an anti-CD20 antibody-doxorubicin conjugate in a CD20-expressing cell of the same cell type, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, and wherein the rates of accumulation of the anti-CD20 antibody-cytotoxic agent conjugate and of the anti-CD20 antibody-doxorubicin conjugate are measured by a method comprising:
(a) culturing a population of the CD20-expressing cell with the anti-CD20 antibody-cytotoxic agent conjugate;
(b) culturing a population of the CD20-expressing cell with the anti-CD20 antibody-doxorubicin conjugate, wherein the populations of steps (a) and (b) are cultured under the same conditions; and
(c) measuring the amount of the anti-CD20 antibody-cytotoxic agent conjugate and anti-CD20 antibody-doxorubicin conjugate accumulated in the populations of steps (a) and (b), respectively.

100. A kit comprising in a first container, an anti-CD20 antibody, and in a second container, a cytotoxic agent, wherein upon conjugation of the anti-CD20 antibody and the drug, the resulting conjugate exhibits an accumulation in a non-peripheral region inside a CD20-expressing cell that is between 1.5-fold and 5,000-fold greater than the accumulation of an unconjugated form of the anti-CD20 antibody in the CD20-expressing cell, with the proviso that the cytotoxic agent is not a radioisotope or a toxin, wherein the accumulation of the conjugate and of the unconjugated form of the antibody are measured by a method comprising:
(a) culturing a population of the CD20-expressing cell with the conjugate;
(b) culturing a population of the CD20-expressing cell with the unconjugated form of the anti-CD20 antibody; and
(c) detecting by confocal fluorescence microscopy localization of the conjugate and the unconjugated form of the anti-CD20 antibody in the populations of steps (a) and (b), respectively,
wherein the populations of steps (a) and (b) are cultured under the same conditions and for the same period of time, and wherein the conjugate exhibits an accumulation in the CD20-expressing cell that is between 1.5-fold and 5,000-fold greater than the accumulation of the unconjugated form of the anti-CD20 antibody in the CD20-expressing cell if:
(i) between 1.5-fold and 5,000-fold as many cells of the population of step (a) contain a detectable amount of the conjugate in a non-peripheral region as the number of cells of the population of step (b) contain the unconjugated form of the antibody in a non-peripheral region; or
(ii) the accumulation of the conjugate in a non-peripheral region of the majority of CD20-expressing cells of the population of step (a) is between 1.5-fold and 5,000-fold greater than the accumulation of the unconjugated form of the anti-CD20 antibody in the majority of CD20-expressing cells of the population of step (b).

101. A kit comprising in a first container, an anti-CD20 antibody, and in a second container, a cytotoxic agent, wherein upon conjugation of the anti-CD20 antibody and the drug, the resulting conjugate exhibits an accumulation in a non-peripheral region inside a CD20-expressing cell that is between 1.5-fold and 5,000-fold greater than the accumulation of an anti-CD20 antibody-doxorubicin conjugate, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, in the CD20-expressing cell, wherein the accumulation of the conjugate and of the anti-CD20 antibody-doxorubicin conjugate are measured by a method comprising:
(a) culturing a population of the CD20-expressing cell with the conjugate;
(b) culturing a population of the CD20-expressing cell with the anti-CD20 antibody-doxorubicin conjugate; and
(c) detecting by confocal fluorescence microscopy localization of the conjugate and the anti-CD20 antibody-doxorubicin conjugate in the populations of steps (a) and (b), respectively,
wherein the populations of steps (a) and (b) are cultured under the same conditions and for the same period of time, and wherein the conjugate exhibits an accumulation in the CD20-expressing cell that is between 1.5-fold and 5,000-fold greater than the accumulation of the anti-CD20 antibody-doxorubicin conjugate in the CD20-expressing cell if:
(i) between 1.5-fold and 5,000-fold as many cells of the population of step (a) contain a detectable amount of the conjugate in a non-peripheral region as the number of cells of the population of step (b) contain the anti-CD20 antibody-doxorubicin conjugate in a non-peripheral region; or
(ii) the accumulation of the conjugate in a non-peripheral region of the majority of CD20-expressing cells of the population of step (a) is between 1.5-fold and 5,000-fold greater than the accumulation of the anti-CD20 antibody-doxorubicin conjugate in the majority of CD20-expressing cells of the population of step (b).

102. A kit comprising in a first container, an anti-CD20 antibody, in a second container, a cytotoxic agent, and in a third container, a linker for conjugating the anti-CD20 antibody to the cytotoxic agent, wherein upon conjugation of the anti-CD20 antibody and the drug via the linker, the resulting conjugate has a rate of accumulation in a CD20-expressing cell that is between 20-fold and 5,000-fold greater than the rate of accumulation of an unconjugated form of the anti-CD20 antibody in the CD20-expressing cell, with the proviso that the cytotoxic agent is not a radioisotope or a toxin, and wherein the rates of accumulation of the conjugate and of the unconjugated form of the antibody are measured by a method comprising:
(a) culturing a population of the CD20-expressing cell with the conjugate;
(b) culturing a population of the CD20-expressing cell with the unconjugated antibody, wherein the populations of steps (a) and (b) are cultured under the same conditions; and
(c) measuring the amount of the conjugate and unconjugated antibody accumulated in the populations of steps (a) and (b), respectively.

103. A kit comprising in a first container, an anti-CD20 antibody, in a second container, a cytotoxic agent, and in a third container, a linker for conjugating the anti-CD20 antibody to the cytotoxic agent, wherein upon conjugation of the anti-CD20 antibody and the drug via the linker, the resulting conjugate exhibits an accumulation in a non-peripheral region inside a CD20-expressing cell that is between 1.5-fold and 5,000-fold greater than the accumulation of an unconjugated form of the anti-CD20 antibody in the CD20-expressing cell, with the proviso that the cytotoxic agent is not a radioisotope or a toxin, wherein the accumulation of the conjugate and of the unconjugated form of the antibody are measured by a method comprising:
(a) culturing a population of the CD20-expressing cell with the conjugate;
(b) culturing a population of the CD20-expressing cell with the unconjugated form of the anti-CD20 antibody; and
(c) detecting by confocal fluorescence microscopy localization of the conjugate and the unconjugated form of the anti-CD20 antibody in the populations of steps (a) and (b), respectively,
wherein the populations of steps (a) and (b) are cultured under the same conditions and for the same period of time, and wherein the conjugate exhibits an accumulation in the CD20-expressing cell that is between 1.5-fold and 5,000-fold greater than the accumulation of the unconjugated form of the anti-CD20 antibody in the CD20-expressing cell if:
(i) between 1.5-fold and 5,000-fold as many cells of the population of step (a) contain a detectable amount of the conjugate in a non-peripheral region as the number of cells of the population of step (b) contain the unconjugated form of the antibody in a non-peripheral region; or
(ii) the accumulation of the conjugate in a non-peripheral region of the majority of CD20-expressing cells of the population of step (a) is between 1.5-fold and 5,000-fold greater than the accumulation of the unconjugated form of the anti-CD20 antibody in the majority of CD20-expressing cells of the population of step (b).

104. A kit comprising in a first container, an anti-CD20 antibody, in a second container, a cytotoxic agent, and in a third container, a linker for conjugating the anti-CD20 antibody to the cytotoxic agent, wherein upon conjugation of the anti-CD20 antibody and the drug via the linker, the resulting conjugate exhibits an accumulation in a non-peripheral region inside a CD20-expressing cell that is between 1.5-fold and 5,000-fold greater than the accumulation of an anti-CD20 antibody-doxorubicin conjugate, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, in the CD20-expressing cell, wherein the accumulation of the conjugate and of the anti-CD20 antibody-doxorubicin conjugate are measured by a method comprising:
(a) culturing a population of the CD20-expressing cell with the conjugate;
(b) culturing a population of the CD20-expressing cell with the anti-CD20 antibody-doxorubicin conjugate; and
(c) detecting by confocal fluorescence microscopy localization of the conjugate and the anti-CD20 antibody-doxorubicin conjugate in the populations of steps (a) and (b), respectively,
wherein the populations of steps (a) and (b) are cultured under the same conditions and for the same period of time, and wherein the conjugate exhibits an accumulation in the CD20-expressing cell that is between 1.5-fold and 5,000-fold greater than the accumulation of the anti-CD20 antibody-doxorubicin conjugate in the CD20-expressing cell if:
(i) between 1.5-fold and 5,000-fold as many cells of the population of step (a) contain a detectable amount of the conjugate in a non-peripheral region as the number of cells of the population of step (b) contain the anti-CD20 antibody-doxorubicin conjugate in a non-peripheral region; or
(ii) the accumulation of the conjugate in a non-peripheral region of the majority of CD20-expressing cells of the population of step (a) is between 1.5-fold and 5,000-fold greater than the accumulation of the anti-CD20 antibody-doxorubicin conjugate in the majority of CD20-expressing cells of the population of step (b).

105. A kit comprising in a first container, an anti-CD20 antibody, in a second container, a cytotoxic agent, and in a third container, a linker for conjugating the anti-CD20 antibody to the cytotoxic agent, wherein upon conjugation of the anti-CD20 antibody and the drug via the linker, the resulting conjugate has a rate of accumulation in a CD20-expressing cell that is between 20-fold and 5,000-fold greater than the rate of accumulation of an anti-CD20 antibody-doxorubicin conjugate in a CD20-expressing cell of the same cell type, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, and wherein the rates of accumulation of the anti-CD20 antibody-cytotoxic agent conjugate and of the anti-CD20 antibody-doxorubicin conjugate are measured by a method comprising:
(a) culturing a population of the CD20-expressing cell with the anti-CD20 antibody-cytotoxic agent conjugate;
(b) culturing a population of the CD20-expressing cell with the anti-CD20 antibody-doxorubicin conjugate, wherein the populations of steps (a) and (b) are cultured under the same conditions; and
(c) measuring the amount of the anti-CD20 antibody-cytotoxic agent conjugate and anti-CD20 antibody-doxorubicin conjugate accumulated in the populations of steps (a) and (b), respectively.

106. A kit comprising:
(a) an anti-CD20 antibody-cytotoxic agent conjugate, wherein the cytotoxic agent of the anti-CD20 antibody-cytotoxic agent conjugate has an IC₅₀ of between 40-fold and 4,000-fold less than the IC₅₀ of doxorubicin, with the proviso that the cytotoxic agent is not a radioisotope or a toxin, and wherein the IC₅₀ of each of the cytotoxic agent and doxorubicin is measured by a method comprising:
(i) culturing one or more CD20-expressing cell populations in the presence of one or more concentrations of the cytotoxic agent for a 72- to 96-hour period;
(ii) culturing one or more CD20-expressing cell populations in the presence of one or more concentrations of doxorubicin for a 72-to 96-hour period; and
(iii) identifying a concentration of the cytotoxic agent and doxorubicin, respectively, at which 50% fewer cells in the CD20-expressing cell populations of steps (i) and (ii), respectively, are viable at the end of the period relative to a CD20-expressing cell population cultured in the absence of the cytotoxic agent and doxorubicin,
wherein the CD20-expressing cell populations of steps (i), (ii) and (iii) are of the same cell type and are cultured under the same conditions,
and wherein the concentration of the cytotoxic agent and doxorubicin identified in step (iii) is the IC₅₀ of the cytotoxic agent and doxorubicin, respectively, and
(b) a notice by a regulatory agency indicating approval for manufacture, use or sale of the conjugate for human administration.

107. A kit comprising:
(a) an anti-CD20 antibody-cytotoxic agent conjugate, wherein the anti-CD20 antibody-cytotoxic agent conjugate has an IC₅₀ of between 40-fold and 4,000-fold less than the IC₅₀ of an anti-CD20 antibody-doxorubicin conjugate, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, and wherein the IC₅₀ of each of the anti-CD20 antibody-cytotoxic agent conjugate and the anti-CD20 antibody-doxorubicin conjugate is measured by a method comprising:
(i) culturing one or more CD20-expressing cell populations in the presence of one or more concentrations of the anti-CD20 antibody-cytotoxic agent conjugate for a 72- to 96-hour period;
(ii) culturing one or more CD20-expressing cell populations in the presence of one or more concentrations of the anti-CD20 antibody-doxorubicin conjugate for a 72- to 96-hour period; and
(iii) identifying a concentration of the anti-CD20 antibody-cytotoxic agent conjugate and the anti-CD20 antibody-doxorubicin conjugate, respectively, at which 50% fewer cells in the CD20-expressing cell populations of steps (i) and (ii), respectively, are viable at the end of the period relative to a CD20-expressing cell population cultured in the absence of the anti-CD20 antibody-cytotoxic agent conjugate and the anti-CD20 antibody-doxorubicin conjugate,
wherein the CD20-expressing cell populations of steps (i), (ii) and (iii) are of
the same cell type and are cultured under the same conditions,
and wherein the concentration of the anti-CD20 antibody-cytotoxic agent conjugate and the anti-CD20 antibody-doxorubicin conjugate identified in step (iii) is the IC₅₀ of the anti-CD20 antibody-cytotoxic agent conjugate and the anti-CD20 antibody-doxorubicin conjugate, respectively; and
(b) a notice by a regulatory agency indicating approval for manufacture, use or sale of the conjugate for human administration.

108. A kit comprising:
(a) an anti-CD20 antibody-cytotoxic agent conjugate, wherein the conjugate has a rate of accumulation in a CD20-expressing cell that is between 20-fold and 5,000-fold greater than the rate of accumulation of an unconjugated form of the anti-CD20 antibody in the CD20-expressing cell, with the proviso that the cytotoxic agent is not a radioisotope or a toxin, and wherein the rates of accumulation of the conjugate and of the unconjugated form of the antibody are measured by a method comprising:
(i) culturing a population of the CD20-expressing cell with the conjugate;
(ii) culturing a population of the CD20-expressing cell with the unconjugated antibody, wherein the populations of steps (i) and (ii) are cultured under the same conditions; and
(iii) measuring the amount of the conjugate and unconjugated antibody accumulated in the populations of steps (i) and (ii), respectively; and
(b) a notice by a regulatory agency indicating approval for manufacture, use or sale of the conjugate for human administration.

109. A kit comprising:
(a) an anti-CD20 antibody-cytotoxic agent conjugate, wherein the conjugate has a rate of accumulation in a CD20-expressing cell that is between 20-fold and 5,000-fold greater than the rate of accumulation of an anti-CD20 antibody-doxorubicin conjugate in a CD20-expressing cell of the same cell type, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, and wherein the rates of accumulation of the anti-CD20 antibody-cytotoxic agent conjugate and of the anti-CD20 antibody-doxorubicin conjugate are measured by a method comprising:
(i) culturing a population of the CD20-expressing cell with the anti-CD20 antibody-cytotoxic agent conjugate;
(ii) culturing a population of the CD20-expressing cell with the anti-CD20 antibody-doxorubicin conjugate, wherein the populations of steps (i) and (ii) are cultured under the same conditions; and
(iii) measuring the amount of the anti-CD20 antibody-cytotoxic agent conjugate and anti-CD20 antibody-doxorubicin conjugate accumulated in the populations of steps (i) and (ii), respectively; and
(b) a notice by a regulatory agency indicating approval for manufacture, use or sale of the conjugate for human administration.

110. A kit comprising:
(a) an anti-CD20 antibody-cytotoxic agent conjugate, wherein the conjugate exhibits an accumulation in a non-peripheral region inside a CD20-expressing cell that is between 1.5-fold and 5,000-fold greater than the accumulation of an unconjugated form of the anti-CD20 antibody in the CD20-expressing cell, with the proviso that the cytotoxic agent is not a radioisotope or a toxin, wherein the accumulation of the conjugate and of the unconjugated form of the antibody are measured by a method comprising:
(i) culturing a population of the CD20-expressing cell with the conjugate;
(ii) culturing a population of the CD20-expressing cell with the unconjugated form of the anti-CD20 antibody; and
(iii) detecting by confocal fluorescence microscopy localization of the conjugate and the unconjugated form of the anti-CD20 antibody in the populations of steps (a) and (b), respectively,
wherein the populations of steps (a) and (b) are cultured under the same conditions and for the same period of time, and wherein the conjugate exhibits an accumulation in the CD20-expressing cell that is between 1.5-fold and 5,000-fold greater than the accumulation of the unconjugated form of the anti-CD20 antibody in the CD20-expressing cell if:
(A) between 1.5-fold and 5,000-fold as many cells of the population of step (a) contain a detectable amount of the conjugate in a non-peripheral region as the number of cells of the population of step (b) contain the unconjugated form of the antibody in a non-peripheral region; or
(B) the accumulation of the conjugate in a non-peripheral region of the majority of CD20-expressing cells of the population of step (a) is between 1.5-fold and 5,000-fold greater than the accumulation of the unconjugated form of the anti-CD20 antibody in the majority of CD20-expressing cells of the population of step (ii); and
(b) a notice by a regulatory agency indicating approval for manufacture, use or sale of the conjugate for human administration.

111. A kit comprising:
(a) an anti-CD20 antibody-cytotoxic agent conjugate, wherein the conjugate exhibits an accumulation in a non-peripheral region inside a CD20-expressing cell that is between 1.5-fold and 5,000-fold greater than the accumulation of an anti-CD20 antibody-doxorubicin conjugate, with the proviso that the cytotoxic agent is not a radioisotope or a toxin, wherein the anti-CD20 antibody-cytotoxic agent conjugate and the anti-CD20 antibody-doxorubicin conjugate comprise the same anti-CD20 antibody, in the CD20-expressing cell, wherein the accumulation of the conjugate and of the anti-CD20 antibody-doxorubicin conjugate are measured by a method comprising:
(i) culturing a population of the CD20-expressing cell with the conjugate;
(ii) culturing a population of the CD20-expressing cell with the anti-CD20 antibody-doxorubicin conjugate; and
(iii) detecting by confocal fluorescence microscopy localization of the conjugate and the anti-CD20 antibody-doxorubicin conjugate in the populations of steps (a) and (b), respectively,
wherein the populations of steps (a) and (b) are cultured under the same conditions and for the same period of time, and wherein the conjugate exhibits an accumulation in the CD20-expressing cell that is between 1.5-fold and 5,000-fold greater than the accumulation of the anti-CD20 antibody-doxorubicin conjugate in the CD20-expressing cell if:
(A) between 1.5-fold and 5,000-fold as many cells of the population of step (a) contain a detectable amount of the conjugate in a non-peripheral region as the number of cells of the population of step (b) contain the anti-CD20 antibody-doxorubicin conjugate in a non-peripheral region; or
(B) the accumulation of the conjugate in a non-peripheral region of the majority of CD20-expressing cells of the population of step (a) is between 1.5-fold and 5,000-fold greater than the accumulation of the anti-CD20 antibody-doxorubicin conjugate in the majority of CD20-expressing cells of the population of step (ii); and
(b) a notice by a regulatory agency indicating approval for manufacture, use or sale of the conjugate for human administration.

112-121. (canceled)
